SEQUENCE LISTING (20 sequences)

SEQ ID NO: 1 - DNA, 24 bp, Artificial
  MADE IN LAB
atggcnaayggnacngaygcnagy

SEQ ID NO: 2 - DNA, 27 bp, Artificial
  MADE IN LAB
ytgngtnayccaytgrtcngtnccngc

SEQ ID NO: 3 - DNA, 28 bp, Artificial
  MADE IN LAB
atcgggatccatggccaacgggaccaac

SEQ ID NO: 4 - DNA, 28 bp, Artificial
  MADE IN LAB
gtacgaattccctcatctgcggggaggc

SEQ ID NO: 5 - DNA, 31 bp, Artificial
  MADE IN LAB
atcgggatccatggccaacgggaccgacgcc

SEQ ID NO: 6 - DNA, 54 bp, Artificial
  MADE IN LAB
atcggaattctcacttgtcatcgtcgtccttgtagtcggggagagccaggggcc

SEQ ID NO: 7 - protein, 12 aa, Mus musculus
MANGTDASVPLT

SEQ ID NO: 8 - protein, 13 aa, Mus musculus
AGTDQWLTQQSPS

SEQ ID NO: 9 - DNA, 783 bp, Mus musculus
gaaaaaaagccacagtcatggccaacgggaccgacgcctctgtcccgctcaccagctatgagtattacctggactacatagacctcattcctgtggacgagaagaagctgaaagccaacaagcattccattgtcatcgccctgtggttgagcctggctaccttcgtggtgctcctctttctcatcctgctctacatgtcctggtcgggctccccacagatgaggcacagtccccaaccccagccaatatgttcatggactcacagcttcaacctccctctgtgcctccggagggcctccctgcagacaacagaggagccaggaaggagagctggcactgaccagtggttaacgcagcagagtccttctgcctcagccccggggcccctggctctcccctaggaccaggtccaggatggaggtcccagggcatcagctggcctcacactcaagcagtggtgagcctggagacagagcgtctcaactgtagaacggatgatgccagagagccagtcgggctcaagcaaacggtgaactccaaccaacccgggcagctacgtcttttttagggccgtttacaatggccttgaatatagcaggaaactgaccgggacaaaaccaagtttacaaagaggaccatcacacacattgatagtgcagctaggatgcaggagctgccctggacacagctgtctctgttgagcaagcttagcctgcttgctgcttacatttgctttgggggtacacaggaaaataaaatgtgaattaggataaaaaaaaaaaa

SEQ ID NO: 10 - DNA, 384 bp, Mus musculus
atggccaacgggaccgacgcctctgtcccgctcaccagctatgagtattacctggactacatagacctcattcctgtggacgagaagaagctgaaagccaacaagcattccattgtcatcgccctgtggttgagcctggctaccttcgtggtgctcctctttctcatcctgctctacatgtcctggtcgggctccccacagatgaggcacagtccccaaccccagccaatatgttcatggactcacagcttcaacctccctctgtgcctccggagggcctccctgcagacaacagaggagccaggaaggagagctggcactgaccagtggttaacgcagcagagtccttctgcctcagccccggggcccctggctctcccctag

SEQ ID NO: 11 - protein, 127 aa, Mus musculus [sequence omitted]

SEQ ID NO: 12 - DNA, 341 bp, Mus musculus
atggccaacgggaccgacgcctctgtcccgctcaccagctatgagtattacctggactacatagacctcattcctgtggacgagaagaagctgaaagccaacaagcgtaagtcggaacacaggaaggtgaccaggcagaggctggggctggggctgagccgggggtgaggccctcttggctttgcccgtcccttactaactctggcagacatgctcagcagggttaacatggctggtgctcacacagacataagcgaatcaggcatgggactcaaactgtatctgtaactttcagtagtaaactttctctgagttaacttgccaaaaaaaaaaaaaaaaaa

SEQ ID NO: 13 - DNA, 168 bp, Mus musculus
atggccaacgggaccgacgcctctgtcccgctcaccagctatgagtattacctggactacatagacctcattcctgtggacgagaagaagctgaaagccaacaagcgtaagtcggaacacaggaaggtgaccaggcagaggctggggctggggctgagccgggggtga

SEQ ID NO: 14 - protein, 55 aa, Mus musculus
MANGTDASVPLTSYEYYLDYIDLIPVDEKKLKANKRKSEHRKVTRQRLGLGLSRG

SEQ ID NO: 15 - DNA, 839 bp, Homo sapiens
atggccaacgggaccaacgcctctgccccatactacagctatgaatactacctggactatctggacctcattcccgtggacgagaagaagctgaaagcccacaaacattccatcgtgatcgcattctgggtgagcctggctgccttcgtggtgctgctcttcctcatcttgctctacatgtcctggtccgcctccccgcagatgaggaggaacagccccaagcaccaccaaacatgcccctggagtcacggcctcaacctccacctctgcatccagaagtgcctgccgtgccacagggaacccctggcaacctcacaggctcaggcgagctcagtggagccagggagcagaactggccctgaccagccgctacgacaggagagctcctccaccttgcccctcgggggtttccagacccaccccactctcctctgggaactgaccctcaatgggggtcccctcgtcaggagcaagcccagcgagcctccccctggagacaggacctctcaattgcagagctgatgtcagtaaatcgtggccatagctgagtgaactggtgaaatcaagccaacctggacacatacgttcctcgttcttcttagaggccatttgcatgtagcagaaagggcacctaggtcaagtgcaactagagcaggagcatcctatgcctttgacaaagattgcagtggcccctcgagtgcagaggtcatcccaggtgttgctgagtttattgagcacacctagcctgcttgcttactgcttatatttgctcagggaagagtaggaaaataaaatatatgcaaatcaagaggaaaaaaaaaaaaaaaaaaaaaaaaaaa

SEQ ID NO: 16 - DNA, 522 bp, Homo sapiens
atggccaacgggaccaacgcctctgccccatactacagctatgaatactacctggactatctggacctcattcccgtggacgagaagaagctgaaagcccacaaacattccatcgtgatcgcattctgggtgagcctggctgccttcgtggtgctgctcttcctcatcttgctctacatgtcctggtccgcctccccgcagatgaggaggaacagccccaagcaccaccaaacatgcccctggagtcacggcctcaacctccacctctgcatccagaagtgcctgccgtgccacagggaacccctggcaacctcacaggctcaggcgagctcagtggagccagggagcagaactggccctgaccagccgctacgacaggagagctcctccaccttgcccctcgggggtttccagacccaccccactctcctctgggaactgaccctcaatgggggtcccctcgtcaggagcaagcccagcgagcctccccctggagacaggacctctcaattgcagagctga

SEQ ID NO: 17 - protein, 173 aa, Homo sapiens
MANGTNASAPYYSYEYYLDYLDLIPVDEKKLKAHKHSIVIAFWVSLAAFVVLLFLILLYMSWSASPQMRRNSPKHHQTCPWSHGLNLHLCIQKCLPCHREPLATSQAQASSVEPGSRTGPDQPLRQESSSTLPLGGFQTHPTLLWELTLNGGPLVRSKPSEPPPGDRTSQLQS

SEQ ID NO: 18 - DNA, 469 bp, Homo sapiens
atggccaacgggaccaacgcctctgccccatactacagctatgaatactacctggactatctggacctcattcccgtggacgagaagaagctgaaagcccacaaacattccatcgtgatcgcattctgggtgagcctggctgccttcgtggtgctgctcttcctcatcttgctctacatgtcctggtccgcctccccgcagatgagctttaacacagatgaatctcttctgcattcagaagtgctgcctcaaactcgagctatttcctgtgatgagctccaagcccctagagaggaaggggcggcctgacgaggcactggatgggcctcatggcctagaagtccccacattccttgcaactctgatgctgggcacctacaggcccgagtttatcatttactgggcagttcaataaagtcctctcaacttttcttgtcaaaaaaaaaaaaaaaaaaaaaaaaaaaaaaaa

SEQ ID NO: 19 - DNA, 309 bp, Homo sapiens
atggccaacgggaccaacgcctctgccccatactacagctatgaatactacctggactatctggacctcattcccgtggacgagaagaagctgaaagcccacaaacattccatcgtgatcgcattctgggtgagcctggctgccttcgtggtgctgctcttcctcatcttgctctacatgtcctggtccgcctccccgcagatgagctttaacacagatgaatctcttctgcattcagaagtgctgcctcaaactcgagctatttcctgtgatgagctccaagcccctagagaggaaggggcggcctga

SEQ ID NO: 20 - protein, 102 aa, Homo sapiens
MANGTNASAPYYSYEYYLDYLDLIPVDEKKLKAHKHSIVIAFWVSLAAFVVLLFLILLYMSWSASPQMSFNTDESLLHSEVLPQTRAISCDELQAPREEGAA

(19) United States
(12) Patent Application Publication
(10) Pub. No.: US 2005/0074756 A1
(43) Pub. Date: Apr. 7, 2005

(54) FALP proteins

(76) Inventors: Garth James Smith Cooper, Auckland (NZ); Aimin Xu, Auckland (NZ); Yu Wang, Auckland (NZ)

Correspondence Address: Bradford J. Duft;Buchanan Ingersoll Professional Corporation, One Oxford Centre, 301 Grant Street, Pittsburgh, PA, 15219-1410, US

(21) Appl. No.: 10/378,544

(22) Filed: Mar. 3, 2003

Related U.S. Application Data
(60) Provisional application No. 60/361,152, filed on Mar. 1, 2002.

(51) Int. Cl. C12Q 1/68; C07H 21/04; C12N 9/00; C12P 21/02; C12N 1/21; C12N 15/74

(52) U.S. Cl. 435/6; 435/69.1; 435/252.3; 435/320.1; 435/183; 536/23.2

(57) ABSTRACT

Novel fat tissue low molecular weight proteins (FALPs) that show response to insulin are described.

### RELATED APPLICATIONS

This application claims priority from U.S. Provisional Patent Application Ser. No. 60/361,152, filed Mar. 1, 2002, the contents of which are hereby incorporated in their entirety by reference.

### FIELD OF THE INVENTION

This invention concerns particular adipocyte-related proteins, fragments, variants, and derivatives thereof. This invention further concerns compositions comprising such proteins, methods of making such proteins and compositions, and methods of using such proteins and compositions, for example, to identify and assess compounds that modulate activity.

### BACKGROUND OF THE INVENTION

The following description includes information that may be useful in understanding the present invention. It is not an admission that any of the information provided herein is prior art, or relevant, to the presently described or claimed inventions, or that any publication or document that is specifically or implicitly referenced is prior art.

Adipose tissue has recently reported shown to play an important role in the regulation of energy metabolism, and its malfunction is reportedly a significant cause of insulin resistance and its associated morbidity, such as type 2 diabetes and various cardiovascular diseases. Bergman, R. N., et al., Journal of Investigative Medicine 49, 119-26 (2001); Hotamisligil, G. S., International Journal of Obesity & Related Metabolic Disorders 24, S23-7 (2000). A close correlation has been reported to exist between changes in fat mass and insulin sensitivity, Kahn, B. B. and Flier, J. S., Journal of Clinical Investigation 106, 473-81(2000), and insulin resistance and hyperinsulinemia have been reported to be present in obese as well as in lipodystrophic individuals. Reue, K. and Peterfy, M. Current Atherosclerosis Reports 2, 3906 (2000); Hegele, R. A., Current Atherosclerosis Reports 2, 397-404 (2000). A pivotal role of adipocytes in the regulation of insulin sensitivity has also been proposed to be supported by two recent independent genetic studies on fat-ablated mice that have severe insulin resistance and hyperglycemia Moitra, J., et al., Genes & Development 12, 3168-81 (1998); Shimomura, L, et al., Genes & Development 12, 3182-94(1998). Insulin-sensitizing drugs that target transcriptional regulation of adipocytes have also been said to link insulin sensitivity to adipocyte function. Spiegelman, B. M., Diabetes 47, 507-14 (1998).

Studies of adipocytes became more widespread with the availability of immortal cell lines that differentiate in vitro into white adipocytes. Cornelius, P., et al., Annual Review of Nutrition 14, 99-129 (1994). When cultured in defined media, 3T3 L1 preadipocytes deposit triglyceride in cytoplasmic lipid droplets and express genes that are also expressed in adipocytes in vivo. Various genetically-based approaches have led to reports of the identification of various transcription factors in the complex transcriptional cascade that are said to be activated during adipose conversion. Rosen, E. D. and Spiegelman, B. M., Annual Review of Cell & Developmental Biology 16, 145-71 (2000); Wu, Z., et al., Current Opinion in Cell Biology 11, 689-94 (1999). Such factors include peroxisome proliferator-activated receptor γ (PPAR γ), CCAAT/enhancer binding protein (C/EBP), and adipocyte differentiation and determination factor 1 (ADD1)-sterol regulatory element binding protein 1c (SREBP1c). Sequential activation of these transcription factors has been reported to induce expression of adipocyte-specific genes, including enzymes, structural proteins, hormone receptors and a variety of secreted factors involved in paracrine and endocrine functions. Sorisky, A., Critical Reviews in Clinical Laboratory Sciences 36, 1-34 (1999); Takahashi, M., Nippon Rinsho—Japanese Journal of Clinical Medicine 59, 504-8 (2001).

Molecular characterization of adipocyte-specific genes has previously been based on genetic approaches, such as differential hybridization, subtractive cloning, shotgun techniques and microarray analysis, Baulande, S., et al., Journal of Biological Chemistry 276, 33336-44 (2001); Scherer, P. E., et al., Journal of Biological Chemistry 270, 26746-26749 (1995); Soukas, A., et al., Journal of Biological Chemistry 276, 34167-74 (2001), and adipose tissue has recently been discussed as an endocrine tissue that secretes a variety of proteins (adipocytokines), such as leptin, adiponectin, adipsin, TNF α and resistin. Fruhbeck, G., et al., American Journal of Physiology—Endocrinology & Metabolism 280, E827-47 (2001); Steppan, C. M. and Lazar, M. A., Trends in Endocrinology & Metabolism 13, 18-23 (2002). Studies both in vivo and in vitro have reportedly demonstrated that secretion of adipocytokines from adipose cells has two compartments, constitutive and regulatory. Bradley, R. L., et al., Recent Progress in Hormone Research 56, 329-58 (2001). Secretion of adipocytokines, such as leptin (Barr, V. A., et al., Endocrinology 138, 4463-72 (1997)), adiponectin (Bogan, J. S. and Lodish, H. F. (1999) Journal of Cell Biology 146, 609-20 and adipsin (Kitagawa, K., Rosen, B. S., Spiegelman, B. M., Lienhard, G. E. and Tanner, L. I. (1989) Biochimica et Biophysica Acta 1014, 83-9), may be enhanced by insulin.

Insulin-dependent intracellular trafficking of membrane organelles have been implicated in many metabolic functions of adipose tissue. Simpson, F., et al., Traffic 2, 2-11 (2001). A recent study proposed the existence of at least three different insulin-regulated vesicular trafficking pathways in this tissue. Holman, G. D. and Sandoval, I. V., Trends in Cell Biology 11, 173-9 (2001). For example, insulin is said to induce intracellular trafficking of GLUT4, a mammalian facilitative glucose transporter that is highly expressed in adipose tissue and striated muscle. Simpson, F., Whitehead, J. P. and James, D. E. (2001) Traffic 2, 2-11. In the basal state, GLUT4 appears to be targeted to an intracellular storage compartment in the perinuclear region. Insulin induces the plasma membrane translocation of GLUT4 through activation of at least two parallel signaling pathways, including PI 3 kinase/Akt/PKC ξ/λ pathway and Cbl/CAP pathway. Simpson, F., et al., Traffic 2, 2-11 (2001). This insulin-regulated trafficking process also involves multiple intracellular organelles and a variety of protein components. Holman, G. D. and Sandoval, I. V., Trends in Cell Biology 11, 173-9 (2001). The intracellular storage compartments of the above-noted adipocytokines and their vesicular trafficking pathways, however, are said to be distinct from those of GLUT4. Millar, C. A., et al., Traffic 1, 141-51 (2000). Recent articles are said to relate to the development of adipocyte-specific proteins such as P3 adrenergic receptor, uncoupling protein and PPAR γ, as pharmaceutical targets. Olefsky, J. M. and Saltiel, A. R., Trends in Endocrinology & Metabolism 11, 362-8 (2000); Wieland, H. A. and Hamilton, B. S., International Journal of Clinical Pharmacology & Therapeutics 39, 406-14 (2001).

It will be appreciated that much effort has been put forward with regard to research into fat cells and tissues, as well as their intracellular components and structures and the functions thereof. The present invention is based on the discovery of new fat-associated proteins.

### SUMMARY OF THE INVENTION

It is an object of this invention to provide useful compositions and methods based on novel fat-associated proteins.

Thus, in one aspect, the invention provides isolated or substantially pure human and other mammalian FALP proteins. Such proteins include those corresponding to human FALP-α (SEQ ID NO:17), human FALP-β (SEQ ID NO:20), murine FALP-α (SEQ ID NO:11), and murine FALP-β (SEQ ID NO:14), as well as other mammalian FALP proteins.

In another aspect, the invention provides isolated or synthetic polynucleotides that encode, or are complementary to a sequence that encodes, a FALP protein, as well as fragments or variants thereof, and specifically includes a polynucleotide encoding a FALP. The polynucleotides of the invention may be operably linked to a promoter or other sequence that enhances expression of the polynucleotide in a cell. Such sequences include cDNA sequences for mouse FALP α (SEQ ID NO:9), mouse FALP β (SEQ ID NO:12), human FALP α (SEQ ID NO:15), and human FALP β (SEQ ID NO:18), as well as other mammalian FALP cDNAs. Such sequences also include coding sequences for mature mouse FALP α (SEQ ID NO:10), mature mouse FALP β (SEQ ID NO:13), mature human FALP α (SEQ ID NO:16), and mature human FALP β (SEQ ID NO:19), as well as other polynucleotides sequences coding for other mammalian FALPs.

Thus, in yet another aspect, the invention provides recombinant vectors (for example, a cloning vector or an expression vector) for replicating and/or expressing a FALP proteins, fragments, variants, and so on.

The invention further provides a host cell (for example, a bacterial cell or a eukaryotic cell, including mammalian and human cells) containing a recombinant FALP polynucleotide of the invention, and provides a method for producing a FALP protein, fragment, variant, or fusion protein of any of the above by culturing a cell containing the recombinant FALP polynucleotide under conditions allowing vector replication of cloning vectors or expression or protein from expression vectors.

In another aspect, the invention provides an isolated, substantially pure, synthetic or recombinant FALP protein, variant, or fragments thereof, including immunogenic fragments, all of which may be labeled or unlabeled. In one aspect the polypeptide has an amino acid sequence identical to one disclosed herein. In another aspect, the polypeptide with an amino acid sequence that differs from that disclosed herein by deletions or conservative substitutions, which is at least about 60%, at least about 70%, at least about 80%, or at least about 90% or more identical to the reference sequence and/or that is immunologically cross-reactive with the full-length a naturally occurring FALP protein. In another aspect, the protein of the invention is a fusion protein in which the FALP protein, variant, or fragment is linked directly or indirectly to another protein or polypeptide.

In another aspect, the invention provides an antibody, or antibody fragment (for example, a Fab fragment or a single chain antibody or single chain Fv), or other binding fragment (for example, produced by phage display) that specifically binds to a FALP protein or related polypeptide of the invention. The antibody may be monoclonal and may bind with an affinity of at least about 10⁸ M⁻¹, for example. The invention also provides an isolated cell or a hybridoma capable of secreting the antibody, antibody fragment, or binding molecule. The antibody, fragment or binding molecule may be human or humanized or chimeric, and may be labeled or unlabeled.

In another aspect the invention provides a method of detecting a FALP gene or polynucleotide in a sample by (a) contacting the sample with a probe that specifically binds the gene or polynucleotide, wherein the probe and the gene or polynucleotide form a complex, and detecting the formation of the complex; or (b) specifically amplifying the gene or polynucleotide in the biological sample, and detecting the amplification product; wherein the formation of the complex or presence of the amplification product is correlated with the presence of the FALP gene or polynucleotide in the biological sample. In one embodiment the gene is a DNA and the probe is an antibody. In a different embodiment, the polynucleotide is an RNA and the probe is a polynucleotide.

In another aspect the invention provides a method of detecting a FALP gene product or other FALP-related polypeptide, for example, a fragment or variant of FALP, in a sample by contacting the sample with a probe that specifically binds the FALP gene product or FALP-related polypeptide, wherein the probe and the FALP gene product or FALP-related polypeptide form a complex, and detecting the formation of the complex; wherein the formation of the complex is correlated with the presence or amount of the FALP gene product or FALP-related polypeptide in the biological sample. In one embodiment the gene product is a FALP or FALP-related polypeptide and probe is an antibody, antibody fragment, etc.

In another aspect, the invention provides a method of identifying a modulator of FALP activity by contacting a composition comprising FALP (for example, a cell expressing a recombinant FALP polypeptide) and a test compound and assaying for a biological effect that occurs in the presence but not absence of the test compound, wherein a test compound that induces a biological effect is identified as a modulator of FALP activity. The invention also provides a method for identifying compounds useful for the treatment, prevention or amelioration of FALP-mediated or FALP-related diseases and conditions by evaluating interactin of the compound with the target FALP. In another aspect, the invention provides a method of treating an FALP-mediated condition (for example, obesity and obesity-related conditions) in a mammal by reducing or increasing the activity or expression of FALP in a cell or tissue in the mammal or administering a modulator of FALP function to the mammal. In another related aspect, the invention provides a process for making a pharmaceutical composition by formulating a modulator of FALP activity (for example, binding) for pharmaceutical use.

The invention further provides, for example, for isolated and synthetic FALP alleles and allelic sequences, and intermediates thereof; isolated and synthetic FALP amino acid sequences, and intermediates thereof; isolated and synthetic FALP amino acid sequences with one or more conservative substitutions, including isolated sequences and intermediates thereof; isolated and synthetic FALP amino acid sequences or nucleotide sequences with one or more deletions, insertions or additions, including isolated sequences and intermediates thereof; isolated and synthetic derivatives of FALP proteins, variants, or fragments, including isolated and synthetic intermediates thereof; synthetic FALP antisense sequences, including isolated antisense sequences and intermediates thereof; FALP cloning vectors, including isolated vectors and intermediates thereof; FALP expression vectors including any desired control elements and/or regulatory sequences (such as, for example, enhancers, promoters, transcription terminators, origins of replication, chromosomal integration sequences, 5′ and 3′ untranslated regions, and so on), including isolated expression vectors and intermediates thereof; processes for transforming cells with FALP vectors; FALP ribozymes; FALP triplex molecules, and synthetic triplex molecules intermediates thereof; FALP RNAi polynucleotides, and synthetic RNAi polynucleotides and intermediates thereof; FALP gene therapy methods; FALP peptidomimetics, including synthetic peptidomimetics and intermediates thereof; FALP oligonucleotides, including synthetic oligonucleotides and intermediates thereof; FALP cell-based assays; FALP probes, including synthetic probes and intermediates thereof; FALP proteins including substantially pure, isolated, synthetic and recombinant FALP proteins, and intermediates thereof; FALP recombinant host cells; FALP immunoassays; proteins that are substantially identical to FALP proteins, including synthetic proteins and intermediates thereof; polynucleotides that are substantially identical to FALP polynucleotides, including synthetic polynucleotides and intermediates thereof; transgenic animals the overexpress at least one FALP; FALP genetic “knockout” animals; FALP transgenic plants; FALP introns and exons; FALP leader sequences; FALP homologues, including synthetic homologues and intermediates thereof; mammalian FALP nucleotide sequences that will hybridize under stringent hybridization conditions to a strand, or its complement, using a sequence of at least about 25 to about 50 or so contiguous nucleotides derived from an appropriate portion of one of the sequences identified herein; molecules corresponding to SEQ ID NOS:1-8; molecules corresponding to SEQ ID NOS:9-20; and, kits containing any of the above noted or elsewhere described or referenced materials.

Before describing the invention in general and in terms of specific embodiments, certain terms used in the context of the describing the invention are set forth. Unless indicated otherwise, the following terms have the following meanings when used herein and in the appended claims. Those terms that are not defined below or elsewhere in the specification shall have their art-recognized meaning.

The term “agonist” refers to a molecule that binds to a FALP so as to modulate its activity. It may increase or decrease absolute FALP activity or, alternatively, influence positively or negatively some other parameter of FALP activity, for example, pH, temperature, or co-factor dependence. A “negative agonist” is a compound that decreases the activity of a protein, while a “positive agonist” is a compound that increases the activity of a protein. An “antagonist” is a compound that competes with another compound for interactions with a protein functional site. Agonists and antagonists include antibodies, small molecules, proteins, lipids, carbohydrates, and other molecules.

The terms “allele” or “allelic sequence,” as used herein, refer to a naturally-occurring alternative form of a gene encoding a FALP polypeptide (i.e., a polynucleotide encoding an FALP polypeptide). Alleles often result from mutations (i.e., changes in the nucleic acid sequence), and sometimes produce altered and/or differently regulated mRNAs or polypeptides whose structure and/or function may or may not be altered. Common mutational changes that give rise to alleles are generally ascribed to natural deletions, additions, or substitutions of nucleotides that may or may not affect the encoded amino acids Each of these types of changes may occur alone, in combination with the others, or one or more times within a given gene, chromosome or other cellular polynucleotide. Any given gene may have no, one or many allelic forms. As used herein, the term “allele” refers to either or both a gene or an mRNA transcribed from the gene.

An “amino acid” is a molecule having the structure wherein a central carbon atom (the “alpha (α)-carbon atom”) is linked to a hydrogen atom, a carboxylic acid group (the carbon atom of which is referred to as a “carboxylcarbon atom”), an amino group (the nitrogen atom of which is referred to as an “amino nitrogen atom”), and a side chain group, R. In the process of being incorporated into a protein, an amino acid loses one or more atoms of its amino and carboxylic groups in a dehydration reaction that links one amino acid to another. As a result, when incorporated into a protein, an amino acid is often referred to as an “amino acid residue.” An amino acid may be derivatized or modified before or after incorporation into a protein (for example, by glycosylation, by formation of cystine through the oxidation of the thiol side chains of two noncontiguous cysteine amino acid residues, resulting in a disulfide covalent bond that frequently plays an important role in stabilizing the folded conformation of a protein, etc.). An amino acid may be one that occurs in nature in proteins, or it may be non-naturally occurring (i.e., is produced by synthetic methods such as solid state and other automated synthesis methods). Examples of non-naturally occurring amino acids include α-amino isobutyric acid, 4-amino butyric acid, L-amino butyric acid, 6-amino hexanoic acid, 2-amino isobutyric acid, 3-amino propionic acid, ornithine, norleusine, norvaline, hydroxproline, sarcosine, citralline, cysteic acid, t-butylglyine, t-butylalanine, phenylylycine, cyclohexylalanine, β-alanine, fluoro-amino acids, including beta and gamma amino acids, designer amino acids (for example, β-methyl amino acids, α-methyl amino acids, No-methyl amino acids), and amino acid analogs in general. Amino acid analogs-refer to compounds that have the same basic chemical structure as a naturally occurring amino acid, ie., an alphα-carbon that is bound to a hydrogen, a carboxyl group, an amino group, and an R group, for example, but have modified R groups (for example, norleucine) or modified peptide backbones, while retaining the same basic chemical structure as a naturally occurring amino acid. Amino acid mimetics refers to chemical compounds that have a structure that is different from the general chemical structure of an amino acid, but that generally function in a manner similar to a naturally occurring amino acid.

In addition to its substitutent groups, two different enantiomeric forms of each amino acid exist, designated D and L. In mammals, only L-amino acids are incorporated into naturally occurring proteins, although the invention contemplates proteins incorporating one or more D- and L-amino acids, as well as proteins comprised of just D- or just L-amino acid residues.

Herein, the following abbreviations may be used for the following amino acids (and residues thereof): alanine (Ala, A); arginine (Arg, R); asparagine (Asn, N); aspartic acid (Asp, D); cyteine (Cys, C); glycine (Gly, G); glutamic acid (Glu, E); glutamine (Gln, Q); histidine (His, H); isoleucine (lie, I); leucine (leu, L); lysine (Lys, K); methionine (Met, M); phenylalanine (Phe, F); proline (Pro, P); serine (Ser, S); threonine (Thr, T); tryptophan (Trp, W); tyrosine (Tyr, Y); and valine (Val, V).

The term “amino acid sequence” refers to an oligopeptide, peptide, polypeptide, or protein sequence, a fragment of any of these, and to naturally occurring or synthetic molecules, as well as to electronic or other representations of foregoing suitable for use in conjunction with a computer, for example.

As will be appreciated, embodiments of the invention may be implemented in silico. In such embodiments, actual physically existing amino acids, peptide fragments, etc. are not employed; instead, electronic or other machine manipulable data forms representing these molecules are used. It is understood that in such embodiments, the foregoing nomenclature, while preferable, need not be used. Instead, any suitable nomenclature for such data forms may be employed.

The term “antibody” refers to intact molecules as well as to fragments thereof, such as Fab, F(ab′)₂, and Fv fragments, which are capable of binding an antigenic determinant (i.e., that portion of a molecule (i.e., an epitope) that makes contact with a particular antibody or other binding molecule). Antibodies include, for example, polyclonal, monoclonal, chimeric, and single chain antibodies, single chain Fvs, Fab fragments, and fragments produced by a Fab expression library.

The term “antisense sequences” refers to polynucleotides having a sequence complementary to a RNA sequence. These terms include nucleic acid sequences that bind to mRNA or portions thereof to block transcription of mRNA by ribosomes. Antisense methods are generally well known in the art (see, for example, PCT publication WO 94/12633, and Nielsen et al., 1991, Science 254: 1497; OLIGONUCLEOTIDES AND ANALOGUES, A PRACTICAL APPROACH, edited by F. Eckstein, IRL Press at Oxford University Press (1991); ANTISENSE RESEARCH AND APPLICATIONS (1993, CRC Press)).

In general, the term “biologically active” refers to a protein having the function, for example, the structural, regulatory, or biochemical function, of a naturally occurring molecule. With regard to a FALP, the term “biologically active” refers to a full length protein or fragment thereof derived from any source that possesses one or more FALP characteristics including measurable FALP activity. Such full length protein or fragments may also show immunological cross reactivity with an antibody (polyclonal or monoclonal) that is raised against, and reacts with, a FALP having the amino acid sequence of a naturally occurring FALP.

A protein's “biochemical activity” refers to a chemical interaction or reaction mediated by or involving the protein. Herein, “biochemical activity” is synonymous with FALP activity in general, or a specific type of FALP activity.

A “cell” means any living cell suitable for the desired application. Cells include eukaryotic and prokaryotic cells. Preferred eukaryotic cells include vertebrate cells such as mammalian cells (for example, human, murine, ovine, porcine, equine, canine, and feline cells), avian cells, fish cells, and invertebrate cells such as insect cells and yeast cells. Preferred prokaryotic cells are bacterial cells.

The term “complementary” generally refers to the natural binding of polynucleotides under permissive salt and temperature conditions by base pairing. For example, the sequence “A-G-T” binds to the complementary sequence “T-C-A”. Complementarity between two single-stranded molecules may be “partial”, such that only some of the nucleic acids bind, or it may be “complete”, such that total complementarity exists between the single stranded molecules. The degree of complementarity between nucleic acid molecules has significant effects on the efficiency and strength of the hybridization between them.

The term “composition” as used herein is intended to encompass a product comprising one or more specified ingredients in specified or other amounts, as well as any product which results, directly or indirectly, from combination of the specified ingredients in such specified or other amounts.

A “compound” is a molecule, and includes small molecules, proteins, carbohydrates, and lipids.

A “compound known to interact” with a protein means a compound that has previously been identified as interacting with a protein or other target.

The term “conservative substitution,” when describing a polypeptide, refers to a change in the amino acid composition of the polypeptide that does not substantially alter the activity of the polypeptide, i.e., substitution of amino acids with other amino acids having similar properties. Conservative substitution tables providing functionally similar amino acids are well known in the art. The following six groups each contain amino acids that are generally understood to represent conservative substitutions for one another: 1) Alanine (A), Serine (S), Threonine (T); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); and 6) Phenylalanine (F), Tyrosine (Y), Tryptophan (W) (see also, Creighton, 1984, Proteins, W. H. Freeman and Company).

In addition to the above-defined conservative substitutions, other modifications of amino acid residues can also result in “conservatively modified variants.” For example, one may regard all charged amino acids as substitutions for each other whether they are positive or negative. In addition, conservatively modified variants can also result from individual substitutions, deletions or additions which alter, add or delete a single amino acid or a small percentage of amino acids, for example, often less than 5%, in an encoded sequence. Further, a conservatively modified variant can be made from a recombinant polypeptide by substituting a codon for an amino acid employed by the native or wild-type gene with a different codon for the same amino acid.

The terms “control elements” or “regulatory sequences” include enhancers, promoters, transcription terminators, origins of replication, chromosomal integration sequences, 5′ and 3′ untranslated regions, with which polypeptides or other biomolecules interact to carry out transcription and translation. For eukaryotic cells, the control sequences will generally include a promoter and preferably an enhancer, for example, derived from immunoglobulin genes, SV40, cytomegalovirus, and a polyadenylation sequence, and may include splice donor and acceptor sequences. Depending on the vector system and host utilized, any number of suitable transcription and translation elements, including constitutive and inducible promoters, may be used. When referring to FALP, a promoter other than that naturally associated with the FALP coding sequence can be referred to as a “heterologous” promoter.

A “deletion” refers to a change in an amino acid or nucleotide sequence due to the absence of one or more amino acid residues or nucleotides. The terms “insertion” or “addition” refer to changes in an amino acid or nucleotide sequence resulting in the addition of one or more amino acid residues or nucleotides, respectively, to a molecule or representation thereof, as compared to a reference sequence, for example, the sequence found in the naturally occurring molecule. A “substitution” refers to the replacement of one or more amino acids or nucleotides by different amino acids or nucleotides, respectively.

The term “derivative” refers to a chemical modification of a polypeptide, polynucleotide, or other molecule. In the context of this invention, a “derivative polypeptide”, for example, one modified by glycosylation, pegylation, or any similar process, retains FALP activity. For example, the term “derivative” of FALP refers to FALP proteins, variants, or fragments that have been chemically modified, as, for example, by addition of one or more polyethylene glycol molecules, sugars, phosphates, and/or other such molecules, where the molecule or molecules are not naturally attached to wild-type FALP polypeptides.

A “derivatized” polynucleotide, oligonucleotide, or nucleic acid generally refers to oligo- and polynucleotides that comprise a derivatized substituent. In some embodiments, the substituent is substantially non-interfering with respect to hybridization to complementary polynucleotides. Derivatized oligo- or polynucleotides that have been modified with appended chemical substituents (for example, by modification of an already synthesized oligo- or poly-nucleotide, or by incorporation of a modified base or backbone analog during synthesis) may be introduced into a metabolically active eukaryotic cell to hybridize with a FALP DNA, RNA, or protein where they produce an alteration or chemical modification to a local DNA, RNA, or protein. Alternatively, a derivatized oligo or polynucleotides may interact with and alter FALP polypeptides, or proteins that interact with FALP DNA or FALP gene products, or alter or modulate expression or function of FALP DNA, RNA or protein. Illustrative attached chemical substituents include: europium (III) texaphyrin, cross-linking agents, psoralen, metal chelates (for example, iron/EDTA chelate for iron catalyzed cleavage), topoisomerases, endonucleases, exonucleases, ligases, phosphodiesterases, photodynamic porphyrins, chemotherapeutic drugs (for example, adriamycin, doxirubicin), intercalating agents, base-modification agents, immunoglobulin chains, and oligonucleotides. Iron/EDTA chelates are chemical substituents often used where local cleavage of a nucleic acid sequence is desired (Hertzberg et al., 1982, J. Am. Chem. Soc. 104: 313; Hertzberg and Dervan, 1984, Biochemistry 23: 3934; Taylor et al., 1984, Tetrahedron 40: 457; Dervan, 1986, Science 232: 464). Illustrative attachment chemistries include: direct linkage, for example, via an appended reactive amino group (Corey and Schultz, 1988, Science 238: 1401, which is incorporated herein by reference) and other direct linkage chemistries, although streptavidin/biotin and digoxigenin/anti-digoxigenin antibody linkage methods can also be used. Examples of methods for linking chemical substituents are provided in U.S. Pat. Nos. 5,135,720, 5,093,245, and 5,055,556, which are incorporated herein by reference. Other linkage chemistries may be used at the discretion of the practitioner.

As used herein, a “detectable laber” has the ordinary meaning in the art and refers to an atom (for example, radionuclide), molecule (for example, fluorescein), or complex, that is or can be used to detect (for example, due to a physical, chemical or optical property), indicate the presence of a molecule or to enable binding of another molecule to which it is covalently bound or otherwise associated. The term “label” also refers to covalently bound or otherwise associated molecules (for example, a biomolecule such as an enzyme) that acts on a substrate to produce a detectable atom, molecule or complex. Detectable labels suitable for use in the present invention include, for example, any composition detectable by spectroscopic, photochemical, biochemical, immunochemical, electrical, optical, chemical, or other means.

The term “epitope” has its ordinary meaning of a site on an antigen or antigenic molecule recognized by an antibody. Epitopes may be segments of amino acids, including segments that represent a small portion of a whole protein or polypeptide. Epitopes may be conformational (i.e., discontinuous). That is, they may be formed from amino acids encoded by noncontiguous parts of a primary sequence that have been juxtaposed by protein folding.

“FALP” refers to a protein, preferably a substantially purified protein, having FALP activity, including those having amino acid sequence according to SEQ ID NOS:11, 14, 17 and 20. While FALPs for use in practicing the present invention may be obtained from any species, particularly preferred are FALPs isolated from mammalian species (for example, bovine, canine, equine, feline, murine, ovine, porcine, equine), most preferably from the human species. In addition, FALPs may be obtained from any source, be it a natural, synthetic, semi-synthetic, or recombinant source. As indicated herein, the present invention also concerns FALP fragments, analogs, variants, and derivatives, and polynucleotides encoding the same. Variants may be a naturally occurring allelic variants or non-naturally occurring variants, and include deletion variants, substitution variants, and addition or insertion variants. As known in the art and set forth herein, an allelic variant is an alternate form of a polynucleotide sequence that may have a substitution, deletion, or addition of one or more nucleotides. Variants also include such naturally occurring variants as splice variants.

The term “fusion protein,” refers to a composite polypeptide, ie., a single contiguous amino acid sequence, made up of two (or more) distinct, polypeptides that fused or otherwise linked together, directly or indirectly, in a single amino acid sequence. Thus, for example, a fusion protein may include a single amino acid sequence that contains two entirely distinct amino acid sequences or two similar or identical polypeptide sequences that are not normally found together in the same configuration in a single amino acid sequence found in nature. Fusion proteins may generally be prepared using either recombinant nucleic acid methods, i.e., as a result of transcription and translation of a recombinant gene fusion product, which fusion comprises a segment encoding a polypeptide of the invention and a segment encoding a heterologous polypeptide, or by chemical synthesis methods well known in the art.

A “functional site” of a protein refers to any site in a protein that has a function. Representative examples include active sites (i.e., those sites in catalytic proteins where catalysis occurs), protein-protein interaction sites, sites for chemical modification (for example, glycosylation and phosphorylation sites), and ligand binding sites. Ligand binding sites include metal ion binding sites, co-factor binding sites, antigen binding sites, substrate channels and tunnels, and substrate binding sites. In an enzyme, a ligand binding site that is a substrate binding site may also be an active site, or overlap with an active site. As used herein, the “biochemical function” of a functional site refers to the function carried out by the site in a naturally occurring protein that possesses the corresponding function. For example, the biochemical function of an active site refers to the specific catalytic activity of the site, whereas the biochemical function of a substrate binding site is the binding of the particular substrate.

The term “gene product” refers to an RNA molecule transcribed from a gene, or a polypeptide encoded by the gene or translated from the RNA.

The term “high affinity” for an IgG antibody, as used herein, refers to an association constant (Ka) of at least about 10⁶M⁻¹, preferably at least about 10⁸M⁻¹, more preferably at least about 10⁹M⁻¹ or greater, for example, up to 10¹²M⁻¹ or greater. However, “high affinity” binding can vary for other antibody isotypes.

A “homologue,” in the context of a polypeptide or nucleic acid molecule, implies an evolutionary relationship, i.e., descendent from a common ancestor.

“Hybridization” refers to any process by which a single-stranded nucleic acid molecule, portion thereof, or single-stranded region of an otherwise double-stranded nucleic acid molecule binds through base pairing with a complementary single-stranded nucleic acid molecule, portion thereof, or single-stranded region of an otherwise double-stranded nucleic acid molecule. Hybridization may be performed where both nucleic acid molecules are in solution, or between one nucleic acid molecule in solution and another nucleic acid molecule immobilized on a solid support (for example, paper, membranes, filters, chips, pins, glass slides, or any other appropriate substrate to nucleic acids can be fixed).

The terms “immunogen” and “immunogenic” have their ordinary meaning in the art, i.e., an immunogen is a molecule, such as a polypeptide or other antigen, that can elicit an adaptive immune response upon introduction into a person or an animal.

A “cell-based assay” is one that employs cells that express a protein of interest. The cell may express the protein endogenously or as a result of recombinant techniques, including the introduction of a suitable expression vector or by introduction (for example, by homologous recombination) of a suitable regulatory element capable of directing the expression of the desired protein. Expression may be constitutive or inducible, as well as transient or stable

An “isolated” molecule (for example, a polypeptide or polynucleotide) refers to a molecule that has been removed from its original environment (for example, the natural environment if it is naturally occurring). For example, a naturally-occurring polynucleotide or polypeptide present in a living animal is not isolated, but the same polynucleotide or polypeptide, separated from some or all of the coexisting materials in the natural system (for example, proteins, lipids, carbohydrates, nucleic acids), is isolated. Such polynucleotides could be part of a vector and/or such polynucleotides or polypeptides could be part of a composition, and still be isolated in that such vector or composition is not part of its natural environment.

The term “modulate” refers to a change in the biochemical activity. For example, modulation may involve an increase or a decrease in catalytic rate, substrate binding characteristics, etc. Modulation may occur, for example, by covalent or non-covalent interaction with the protein, and can involve an increase or decrease in biochemical activity. A “modulator” refers to a compound that causes a change, Le., an increase or decrease, in activity of a protein, and, for example, is typically a ligand, either peptidic, polypeptidic, or small molecule (for example, an agonist or antagonist). A modulator may act directly, for example, by interacting with a protein to cause an increase or decrease in activity. A modulator may also act indirectly, for example, by interfering with, ie., antagonizing or blocking, the action of another molecule that causes an increase or decrease in activity of the protein. The terms “modulator” and “modulation” of FALP activity, as used herein in its various forms, is intended to encompass antagonism, agonism, partial antagonism and/or partial agonism of the activity associated with the FALP protein or gene. In various embodiments, “modulators” may inhibit or stimulate FALP expression or activity. Such modulators include small molecules agonists and antagonists of FALP function or expression, antisense molecules, ribozymes, triplex molecules, and RNAi polynucleotides, gene therapy methods, and others.

The phrases “nucleic acid”, “nucleic acid molecule”, and the like refer to a nucleotide, oligonucleotide, polynucleotide, or any fragment thereof. These phrases also refer to single-stranded or double-stranded DNA and/or RNA of cellular or synthetic origin. In this context, “fragments” refer to those nucleic acid molecules that, when translated, produce polypeptides retaining some functional characteristic, for example, antigenicity or a structural domain of a naturally occurring polypeptide. Unless specifically limited, the disclosure of a polynucleotide sequence is also intended to refer to the complementary sequence. As used herein, the term “polynucleotide” includes oligonucleotides.

As used herein, “obesity” has its ordinary meaning. Obesity is a well established risk factor for a number of potentially life-threatening diseases (obesity-related conditions) such as atherosclerosis, hypertension, diabetes, stroke, pulmonary embolism, and cancer. Furthermore, it complicates numerous chronic conditions such as respiratory diseases, osteoarthritis, osteoporosis, gall bladder disease, and dyslipidemias. The enormity of this problem is best reflected in the fact that death rates escalate with increasing body weight. More than 50% of all cause mortality is attributable to obesity-related conditions once the body mass index (BMI) exceeds 30 kg/m.sup.2, as seen in 35 million Americans. Lee, JAMA 268: 2045-2049 (1992).

The term “oligonucleotide” generally refers to a nucleic acid sequence of at least about 6 nucleotides to about 100 nucleotides, preferably about 15 to about 50 nucleotides, and most preferably about 20 to 40 nucleotides, which can be used, for example, as primers or probes in PCR amplification or in a hybridization assay. As used herein, the term “oligonucleotide” includes “amplimers,” “primers,” “oligomers,” and “probes,” as these terms are commonly used in the art.

The terms “operably associated” and “operably linked” refer to functionally related nucleic acid molecules. For example, a promoter is operably associated with or operably linked to a coding sequence if the promoter assists in control of transcription and/or translation of the encoded polypeptide in an appropriate host cell or other expression system. While operably associated or operably linked nucleic acid molecules can be contiguous and in the same reading frame, certain genetic elements need not be contiguously linked to the nucleic acid encoding the polypeptide(s) to be expressed. For example, enhancers need not be located in close proximity to the coding sequences whose transcription they enhance.

The terms “peptidomimetic” and “mimetic” refer to a synthetic chemical compound that may have substantially the same structural and functional characteristics of the FALP polypeptides of the invention and that mimic FALP structure and/or activity, at least in part and to some degree. Peptide analogs are commonly used in the pharmaceutical industry as non-peptide drugs with properties analogous to those of the template peptide. These types of non-peptide compound are termed “peptide mimetics” or “peptidomimetics” (Fauchere, J. Adv. Drug Res. 15: 29 (1986); Veber and Freidinger TINS p. 392 (1985); and Evans et al. J. Med. Chem. 30: 1229 (1987)). Peptide mimetics that are structurally similar to therapeutically useful peptides may be used to produce an equivalent or enhanced therapeutic or prophylactic effect. Generally, peptidomimetics are structurally similar to a paradigm polypeptide (i.e., a polypeptide that has a biological or pharmacological activity), such as a FALP, but have one or more peptide linkages optionally replaced by a linkage selected from the group consisting of, for example for example, —CH2NH—, —CH2S—, —CH2-CH2-, —CH═CH— (cis and trans), —COCH2-, —CH(OH)CH2-, and —CH2SO—. The mimetic can be either entirely composed of synthetic, non-natural analogues of amino acids, or, is a chimeric molecule of partly natural peptide amino acids and partly non-natural analogs of amino acids. The mimetic can also incorporate any amount of natural amino acid conservative substitutions as long as such substitutions also do not substantially alter the mimetic's structure and/or activity. For example, a mimetic composition is within the scope of the invention if it is capable of carrying out the binding or one or more other biological activities of FALP, for example, enzymatic activity.

The phrase “percent (%) identity” refers to the percentage of sequence similarity found in a comparison of two or more amino acid sequences. Percent identity can be determined electronically using any suitable software. Likewise, “similarity” between two polypeptides (or one or more portions of either or both of them) is determined by comparing the amino acid sequence of one polypeptide to the amino acid sequence of a second polypeptide. Any suitable algorithm useful for such comparisons can be adapted for application in the context of the invention.

By “pharmaceutically acceptable” it is meant, for example, a carrier, diluent or excipient that is compatible with the other ingredients of the formulation and generally safe for administration to a recipient thereof.

The term “polypeptide” is used interchangeably herein with the term “protein,” and refers to a polymer composed of amino acid residues linked by amide linkages, including synthetic, naturally-occurring and non-naturally occurring analogs thereof (amino acids and linkages). Peptides are examples of polypeptides.

A “polynucleotide” means a plurality of nucleotides. Thus, the terms “nucleotide sequence” or “nucleic acid” or “polynucleotide” or “oligonculeotide” are used interchangeably and refer to a heteropolymer of nucleotides or the sequence of these nucleotides. These phrases also refer to DNA or RNA of genomic or synthetic origin which may be single-stranded or double-stranded and may represent the sense or the antisense strand, to peptide nucleic acid (PNA) or to any DNA-like or RNA-like material. It is contemplated that where the polynucleotide is RNA, the T (thymine) in the sequences provided herein is substituted with U (uracil). A polynucleotide that encodes a FALP, a FALP fragment, or a FALP variant refers to a polynucleotide encoding: the mature form of the FALP found in nature; the mature form of the FALP found in nature and additional coding sequence, for example, a leader or signal sequence or a proprotein sequence; either of the foregoing and non-coding sequences (for example, introns or non-coding sequence 5′ and/or 3′ of the coding sequence for the mature form of the polypeptide found in nature); fragments of the mature form of the FALP found in nature; and variants of the mature form of the FALP found in nature. Thus, the term “FALP-encoding polynucleotide” and the like encompass polynucleotides that include only a coding sequence for a desired FALP, fragment, or variant, as well as a polynucleotide that includes additional coding and/or non-coding sequences.

The term “polynucleotide encoding a FALP” and the like refer to a nucleic acid molecule or fragment thereof that (a) has the nucleotide sequence of any of SEQ ID NOS:9, 10, 12, 13, 15, 16, 18 or 19, for example; (b) has a nucleic acid sequence encoding a polypeptide that is at least 85 percent identical, but may be greater than 85 percent, i.e., 86, 87, 88, 89, 90, 91, 92, 93, 94, 95, 96, 97, 98, or 99 percent identical to the polypeptide encoded by any of SEQ ID NOS:110, 13, 16 or 19, for example; (c) is a naturally occurring allelic variant or alternate splice variant of (a) or (b); (d) is a nucleic acid variant of (a)-(c) produced as provided for herein; (e) has a sequence that is complementary to (a)-(d); (f) hybridizes to any of (a)-(e) under conditions of high stringency; and/or (g) has a nucleic acid sequence encoding 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, or up to 100 amino acid substitutions and/or deletions of any naturally occurring FALP.

As used herein, a “probe,” when used in the context of polynucleotides and antibodies, refers to a molecule that specifically binds another molecule. One example of a probe is a “nucleic acid probe,” which can be a DNA, RNA, or other polynucleotide. Where a specific sequence for a nucleic acid probe is given, it is understood that the complementary strand is also identified and included. The complementary strand will work equally well in situations where the target is a double-stranded nucleic acid that specifically binds (for example, anneals or hybridizes) to a substantially complementary nucleic acid. Another example of a probe is an “antibody probe” that specifically binds to a corresponding antigen or epitope.

In general, the term “protein” refers to any polymer of two or more individual amino acids (whether or not naturally occurring) linked via peptide bonds, as occur when the carboxylcarbon atom of the carboxylic acid group bonded to the α-carbon of one amino acid (or amino acid residue) becomes covalently bound to the amino nitrogen atom of the amino group bonded to the α-carbon of an adjacent amino acid. These peptide bond linkages, and the atoms comprising them (i.e., α-carbon atoms, carboxylcarbon atoms (and their substituent oxygen atoms), and amino nitrogen atoms (and their substituent hydrogen atoms)) form the “polypeptide backbone” of the protein. In addition, as used herein, the term “protein” is understood to include the terms “polypeptide” and “peptide” (which, at times, may be used interchangeably herein). Similarly, protein fragments, analogs, derivatives, and variants are may be referred to herein as “proteins,” and shall be deemed to be a “protein” unless otherwise indicated. The term “fragment” of a protein refers to a polypeptide comprising fewer than all of the amino acid residues of the protein. As will be appreciated, a “fragment” of a protein may be a form of the protein truncated at the amino terminus, the carboxy terminus, and/or internally (such as by natural splicing), and may also be variant and/or derivative. A “domain” of a protein is also a fragment, and comprises the amino acid residues of the protein required to confer biochemical activity corresponding to naturally occurring protein. A “variant” or “analog” refers to a protein altered by one or more amino acids in relation to a reference protein (for example, a naturally occurring form of the protein), for example, by one or more amino acid sequence substitutions, deletions, and/or insertions. A variant may have “conservative” changes, wherein a substituted amino acid has similar structural or chemical properties (for example, replacement of leucine with isoleucine). Alternatively, a variant may one or more have “nonconservative” changes (for example, replacement of glycine with tryptophan). Other variations include amino acid deletions or insertions, or both. Such variants can be prepared from corresponding nucleic acid molecule variants, which have a nucleotide sequence that varies accordingly from the nucleotide sequences, for example, for wild-type FALP polypeptides. Unless otherwise indicated, a protein's amino acid sequence (ie., its “primary structure” or “primary sequence”) will be written from amino-terminus to carboxy-terminus. In non-biological systems (for example, those employing solid state synthesis), the primary structure of a protein (which also includes disulfide (cysteine) bond locations) can be determined by the user. In addition to primary structure, proteins also have secondary, tertiary, and, in multisubunit proteins, quaternary structure. “Secondary structure” refers to local conformation of the protein chain, with reference to the covalently linked atoms of the peptide bonds and α-carbon linkages that string the amino acid residues of the protein together. Representative examples of secondary structures include α helices, parallel and anti-parallel β structures, and structural motifs such as helix-turn-helix, β-α-β, the leucine zipper, the zinc finger, the β-barrel, and the immunoglobulin fold. “Tertiary structure” concerns the three-dimensional structure of a protein, including the spatial relationships of amino acid side chains and atoms, and the geometric relationships of different regions of the protein. “Quaternary structure” refers to the structure and non-covalent association of different polypeptide subunits in a multisubunit protein.

The term “recombinant” refers to a polynucleotide synthesized or otherwise manipulated in vitro (for example, “recombinant polynucleotide”), to methods of using recombinant polynucleotides to produce gene products in cells or other biological systems, or to a polypeptide (“recombinant protein”) encoded by a recombinant polynucteotide. Thus, a “recombinant” polynucleotide is defined either by its method of production or its structure. In reference to its method of production, the process refers to use of recombinant nucleic acid techniques, for example, involving human intervention in the nucleotide sequence, typically selection or production. Alternatively, it can be a polynucleotide made by generating a sequence comprising a fusion of two or more fragments that are not naturally contiguous to each other. Thus, for example, products made by transforming cells with any non-naturally occurring vector is encompassed, as are polynucleotides comprising sequence derived using any synthetic oligonucleotide process. Similarly, a “recombinant” polypeptide is one expressed from a recombinant polynucleotide.

A “recombinant host cell” is a cell that contains a vector, for example, a cloning vector or an expression vector, or a cell that has otherwise been manipulated by recombinant techniques to express a protein of interest.

The phrase “selectively hybridizing to” refers to a polynucleotide probe that hybridizes, duplexes or binds to a particular target DNA or RNA sequence at a desired level of reactivity or nonreactivity with non-target molecules when the target sequences are present in a preparation of total cellular DNA or RNA.

A “small molecule” means an organic molecule having a molecular weight of less than about 5,000 daltons. A small molecule may be naturally occurring or synthetic.

The phrase “specifically immunoreactive,” or “specifically binds” when referring to the interaction between an antibody and a protein or polypeptide, refers to an antibody that recognizes and detectably binds with relatively high affinity to the protein of interest, for example, FALP, and which binding may be used to determine the presence or amount of the protein in a heterogeneous population of proteins and/or other biologics. Preferably, under designated or desired immunoassay conditions, the specified antibodies bind to a particular polypeptide and do not bind in a significant or undesirable amount to other polypeptides present in the sample, i.e., that are not undesirably cross-reactive with non-target antigens and/or epitopes. A variety of immunoassay formats may be used to select antibodies that are immunoreactive with a particular polypeptide and have a desired specificity. For example, solid-phase ELISA immunoassays are routinely used to select monoclonal antibodies having a desired immunoreactivity and specificity. See, Harlow, 1988, ANTIBODIES, A LABORATORY MANUAL, Cold Spring Harbor Publications, New York (hereinafter, “Harlow”), for a description of immunoassay formats and conditions that may be used to determine or assess immunoreactivity and specificity. Thus, for example, the terms “specific binding”, “specifically binding”, “specificity”, and the like refer to an interaction between a protein and a modulator (for example, an agonist or an antagonist), an antibody, etc., that is not random. Such interactions are believed to be dependent upon the presence of a particular structure of the protein, for example, an activity domain, the antigenic determinant or epitope, etc. “Selective binding”, “selectivity”, and the like refer the preference of a compound to interact with one molecule as compared to another. Preferably, interactions between compounds, particularly modulators, and proteins are both specific and selective.

The term “stably transformed” refers to a nucleic acid molecule that has been inserted into a host cell and exists in the host cell, either as a part of the host cell genomic DNA or as an independent molecule (for example, extra-chromosomally), and that is maintained and replicated in the parent host cell so that it is passed down through successive generations of the host cell.

A difference is typically considered to be “statistically significant” if the probability of the observed difference occurring by chance (the p-value) is less than some predetermined level. As used herein a “statistically significant difference” refers to a p-value that is less than at least about 5% (<0.05), preferably less than at least about 1% (<0.01) and most preferably preferably less than at least about 0.1% (<0.001).

The term “stringent conditions” refers to conditions that permit hybridization between polynucleotides. Stringent conditions can be defined by salt concentration, the concentration of organic solvent (for example, formamide), temperature, and other conditions well known in the art. In particular, stringency can be increased by reducing the concentration of salt, increasing the concentration of organic solvents, (for example, formamide), or raising the hybridization temperature. For example, stringent salt concentration will ordinarily be less than about 750 mM NaCl and 75 mM trisodium citrate, preferably less than about 500 mM NaCl and 50 mM trisodium citrate, and most preferably less than about 250 mM NaCl and 25 mM trisodium citrate. Low stringency hybridization can be obtained in the absence of organic solvent, for example, formamide, while high stringency hybridization can be obtained in the presence of an organic solvent (for example, at least about 35% formamide, most preferably at least about 50% formamide). Stringent temperature conditions will ordinarily include temperatures of at least about 30° C., more preferably of at least about 37° C., and most preferably of at least about 42° C. Varying additional parameters, for example, hybridization time, the concentration of detergent, for example, sodium dodecyl sulfate (SDS), and the inclusion or exclusion of carrier DNA, are well known to those skilled in the art. Various levels of stringency are accomplished by combining these various conditions as needed, and are within the skill in the art. Stringent hybridization conditions may also be defined by conditions in a range from about 5° C. to about 20° C. or 25° C. below the melting temperature (Tm) of the target sequence and a probe with exact or nearly exact complementarity to the target. As used herein, the melting temperature is the temperature at which a population of double-stranded nucleic acid molecules becomes half-dissociated into single strands. Methods for calculating the Tm of nucleic acids are well known in the art (see, for example, Berger and Kimmel, 1987, METHODS IN ENZYMOLOGY, Vol. 152: Guide To Molecular Cloning Techniques, San Diego: Academic Press, Inc. and Sambrook et al.; supra, (1989) MOLECULAR CLONING: A LABORATORY MANUAL, 2nd Ed., Vols. 1-3, Cold Spring Harbor Laboratory). As indicated by standard references, a simple estimate of the Tm value may be calculated by the equation: Tm=81.5+0.41(% G+C), when a nucleic acid is in aqueous solution at 1 M NaCl (see for example, Anderson and Young, “Quantitative Filter Hybridization” in NUCLEIC ACID HYBRIDIZATION (1985)). Other references include more sophisticated computations which take structural as well as sequence characteristics into account for the calculation of Tm. The melting temperature of a hybrid (and thus the conditions for stringent hybridization) is affected by various factors such as the length and nature (DNA, RNA, base composition) of the probe and nature of the target (DNA, RNA, base composition, present in solution or immobilized, and the like), and the concentration of salts and other components (for example for example, the presence or absence of formamide, dextran sulfate, polyethylene glycol). The effects of these factors are well known and are discussed in standard references in the art, see for example, Sambrook, supra, and Ausubel, supra. Typically, stringent hybridization conditions are salt concentrations less than about 1.0 M sodium ion, typically about 0.01 to 1.0 M sodium ion at pH 7.0 to 8.3, and temperatures at least about 30° C. for short probes (for example, 10 to 50 nucleotides) and at least about 60° C. for long probes (for example for example, greater than 50 nucleotides). As noted, stringent conditions may also be achieved with the addition of destabilizing agents such as formamide, in which case lower temperatures may be employed.

The term “substantially purified” or “isolated” refers to nucleic acids or polypeptides that are removed from their natural environment and are isolated or separated, and are at least about 50% free, preferably 60% free, more preferably at least about 75% free, and most preferably at least about 90% free or more, from other components with which they are naturally associated. Thus, a protein or polypeptide is considered substantially pure when that protein makes up greater than about 50% of the total protein content of the composition containing that protein, and typically, greater than about 60% of the total protein content. More typically, a substantially pure or isolated protein or polypeptide will make up at least 75%, more preferably, at least 90%, of the total protein. Preferably, the protein will make up greater than about 90%, and more preferably, greater than about 95% of the total protein in the composition. When referring to polynucleotides, the terms “substantially pure” or “isolated” generally refer to the polynucleotide separated from contaminants with which it is generally associated, for example, lipids, proteins and other polynucleotides. The substantially pure or isolated polynucleotides of the present invention will be greater than about 50% pure. Typically, these polynucleotides will be more than about 60% pure, more typically, from about 75% to about 90% pure and preferably from about 95% to about 98% pure.

As used herein, the terms “substantially sequence identity,” or “substantially identical” (in the context of comparing two or more polypeptides or polynucleotides) refers to two or more sequences or subsequences that have at least 60%, preferably 80%, most preferably 90%, 95%, 98%, or 99% nucleotide or amino acid residue identity, when compared and aligned for maximum correspondence, as measured using one of the following sequence comparison algorithms or by visual inspection. Two sequences (amino acid or nucleotide) can be compared over their full-length (or, for example, the length of the shorter of the two, if they are of different lengths) or over a subsequence such as at least about 50, about 100, about 200, about 500 or about 1000 contiguous nucleotides or at least about 10, about 20, about 30, about 50 or about 100 contiguous amino acid residues. Substantially identical polypeptides, as used herein, preferably have a common functional activity (for example, biological activity). For sequence comparison, typically one sequence acts as a reference sequence, to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters.

Optimal alignment of sequences for comparison can be conducted, for example, by the local homology algorithm of Smith & Waterman, Adv. Appl. Math. 2: 482 (1981), by the homology alignment algorithm of Needleman & Wunsch, J. Mol. Biol. 48: 443 (1970), by the search for similarity method of Pearson & Lipman, Proc. Natl. Acad. Sci. USA 85: 2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by visual inspection (see generally Ausubel et al., CURRENT PROTOCOLS IN MOLECULAR BIOLOGY, Greene Publishing and Wiley-Interscience, New York (supplemented through 2001). When using any of the aforementioned algorithms, the default parameters for “Window” length. gap penalty, etc., are generally used. Another example of an algorithm suitable for determining percent sequence identity and sequence similarity is the BLAST algorithm, which is described, for example, in Altschul et al., J. Mol. Biol. 215: 403410 (1990). Software for performing BLAST analyses is publicly available through the National Center for Biotechnology Information (http://www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are then extended in both directions along each sequence for as far as the cumulative alignment score can be increased. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLAST program uses as defaults a wordlength (W) of II, the BLOSUM62 scoring matrix (see Henikoff & Henikoff, Proc. Natl. Acad. Sci. USA 89: 10915 (1989)) alignments (B) of 50, expectation (E) of 10, M=S, N=−4, and a comparison of both strands. In addition to calculating percent sequence identity, the BLAST algorithm also performs a statistical analysis of the similarity between two sequences (see, for example, Karlin & Altschul, Proc. Natl. Acad. ScL USA 90: 5873-5787 (1993)). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.1, more preferably less than about 0.01, and most preferably less than about 0.001. A further indication that two nucleic acid sequences or polypeptides are substantially identical is where a first polypeptide (for example, a polypeptide encoded by the first nucleic acid) is immunologically cross reactive with a second polypeptide (for example, a polypeptide encoded by the second nucleic acid). Thus, a polypeptide may typically substantially identical to a second polypeptide, for example, where the two peptides differ only by conservative substitutions. An indication that two nucleic acid sequences are substantially identical is that the two molecules hybridize to each other or to a complementary strand of the other under stringent conditions. Substantial identity exists when the segments will hybridize under stringent hybridization conditions to a strand, or its complement, in general using a sequence of at least about 25 to about 50 or so contiguous nucleotides derived from the probe nucleotide sequences.

A molecule (for example, a nucleic acid, protein, or small molecule) that is “synthetic” is one that is produced in whole or in part by chemical synthesis methods.

A “target protein” refers to a protein used in a discovery process, and/or to a therapeutic target. In general, target proteins are used in screening assays to identify compounds that modulate the activity of the protein.

A “test compound” refers to a compound to be tested in an assay.

The term “therapeutically effective amount” means the amount of the subject compound that will elicit a desired response, for example, a biological or medical response of a tissue, system, animal or human that is sought, for example, by a researcher, veterinarian, medical doctor, or other clinician.

“Transformation” describes a process by which an exogenous nucleic acid molecule enters a recipient cell. Transformation may occur under natural or artificial conditions according to various methods well known in the art, and may rely on any known method for the insertion of exogenous nucleic acid molecules into a prokaryotic or eukaryotic host cell. The method for transformation is selected based on the type of host cell being transformed and may include viral infection, calcium phosphate precipitation, electroporation, heat shock, lipofection, and particle bombardment. A “transformed” cell includes stably transformed cells in which the inserted DNA is capable of replication either as an autonomously replicating plasmid or as part of the host chromosome, as well as transiently transformed cells in which the inserted nucleic acid molecule may not replicate or segregate.

The term “vector” refers to a nucleic acid molecule amplification, replication, and/or expression vehicle in the form of a plasmid, phage, viral, or other system (be it naturally occurring or synthetic) for the delivery of nucleic acids to cells where the plasmid, phage, or virus may be functional with bacterial, yeast, invertebrate, and/or mammalian host cells. The vector may remain independent of host cell genomic DNA or may integrate in whole or in part with the genomic DNA. The vector will generally but need not contain all necessary elements so as to be functional in any host cell it is compatible with. An “expression vector” is a vector capable of directing the expression of an exogenous polynucleotide, for example, a polynucleotide encoding a FALP, under appropriate conditions.

### BRIEF DESCRIPTION OF THE DRAWINGS

FIG. 1. Two dimensional gel electrophoresis (2-DE) separation of a low molecular weight protein induced after conversion of 3T3 L1 preadipocytes to adipocytes. Equal amount of proteins from preadipocytes or adipocytes (day 8 after differentiation) were separated by 2-DE and stained with silver. The arrows denote the proteins which were shown to exist in adipocytes and not in preadipocytes.

FIG. 2. Nucleotide and amino acid sequences of mouse FALP (Genbank accession number AY079153), now designated mouse FALP-α. The amino acid sequence is derived from the longest open reading frame. Amino acid sequences for two tryptic peptides determined by amino acid sequencing are underlined. The highlighted amino acid residues between 38 and 61 represent a proposed hydrophobic transmembrane domain, as predicted by TMpred program.

FIG. 3. Time dependent expression of FALP during differentiation induction of 3T3 L1 cell lines. Cells were grown in DMEM with 10% FCS. Confluent preadipocytes (day 0) were exposed to a differentiation mixture as described in the Methods. 10 μg of total RNA prepared from cells at the indicated time points were subjected to Northern blot analysis for mouse FALP and adiponectin and 18 S RNA.

FIG. 4. Tissue distribution of FALP mRNA expression. Northern blot analysis was performed with RNA derived from a variety of mouse tissues as indicated. Full-length cDNA of mouse FALP was used as a probe.

FIG. 5. Cloning of two isoforms of FALP gene from human fat tissue. Total RNA purified from human fat tissue was used as a template to generate cDNA by reverse transcription. The cDNA was then used as a template for PCR analysis. The sense and antisense primers were designed as described in the Methods. Lane 1:1 kb plus DNA ladder. Lane 2.

PCR using two ““guessmers” as primers. Lane 3, PCR using the sense “guessmer” as the specific upstream primer and GeneRacer™ 3′ primer as the downstream primer, as per manufacturer's instructions (Invitrogen).

FIG. 6. The gene organization and transcripts of human FALP a and human FALP β. The upper panel represents the nucleotide sequence and deduced amino acid sequence of two FALP isoforms cloned from human fat tissue. The underlined letters are nucleotide and amino acid sequence shared by both isoforms. The highlighted amino acid residues between 38 and 61 represent predicated hydrophobic transmembrane domains. The bottom graph illustrates the gene structure of human FALP. For a isoform, the sizes of intron I and intron II are ˜7.5 kb and ˜6.0 kb respectively. The intron II for β isoform is ˜8.0 kb.

FIG. 7. Immunolocalization of FLAG-tagged FALP in basal and insulin-treated cells. A: COS 7 cells (A) or 3T3 L1 adipocytes at day 6 after differentiation induction (B and C) were transfected with plasmid pC-FALP-F using Fugene 6 (Roche), and grown in DMEM containing 0.1% FBS for another 48 hr. The cells were treated with 50 nM insulin for 15 min (C), or untreated (A and B). After fixation with methanol/acetone for 2 min, the specimen was sequentially stained with mouse anti-FLAG monoclonal antibody (27 μg/ml) and cy3-conjugated goat anti-mouse polyclonal antibody (1:500), and then visualized under fluorescent microscopy. Expression of FALP was mainly restricted to a compact structure at the perinuclear region, and was redistributed into numerous discrete spots throughout cytoplasm following treatment of 3T3 L1 adipocytes with insulin.

### DETAILED DESCRIPTION OF THE INVENTION

The invention is related to and describes the molecular identification and cloning of novel proteins. The proteins have been shown to be selectively expressed during adipose conversion. The proteins is termed “FALPs.” They are putative integral membrane proteins although their function has not been confirmed. Human and mouse homologues of FALP have been discovered that each exist as two alternatively spliced isoforms, human FALP a, human FALP β, mouse FALP α, and mouse FALP β. Sequence homology searching reveals that FALP does not share sequence identity with any genes with known functions. Given the specific adipose tissue expression and its responsiveness to insulin, it is believed that FALP may represent a novel class of proteins involved in adipose tissue-mediated regulation of energy metabolism. Given adipose tissue specific expression of FLAP, its localization at a dynamic intracellular membrane compartment and its responsiveness to insulin, it appears that the function of FALP is related with intracellular trafficking pathways of adipose cells, such as GLUT4 translocation and hormone secretion.

FALP is also believed to be a target for intervention in treatment of obesity, obesity related conditions, as well as conditions related to intracellular trafficking pathways of adipose cells, such as GLUT4 translocation and hormone secretion. Thus, in one aspect, the invention provides methods for treatment of the aforementioned conditions by down regulating the expression or activity of FALP. In a related embodiment, the invention provides methods of screening for agents useful for treatment the aforementioned conditions by contacting a composition comprising a FALP (for example, a cell comprising a FALP) with an agent cell and detecting a change in activity or expression of the FALP in response to the agent. The treatment of conditions, disorders or diseases characterized by a partial, relative, or absolute FALP deficiency by administration of an appropriate FALP protein, fragment, variant, or derivative, or a positive FALP agonist is also contemplated.

Novel fat tissue low molecular weight proteins (FALPs) that show response to insulin are described A protein of about 14 kDa (with pI value of about 6.2) is differentially expressed in adipocytes and not in preadipocytes, as shown by 2 DE analysis. Northern blot analysis shows the FALP gene is predominantly expressed in brown and white fat tissues, but not in any other tissues examined. Human homologues of mouse FALP have been discovered to exist as two alternatively spliced isoforms, which share the same N-terminus but have a different C-terminus. Mouse FALPs have also been discovered to exist as two alternative isoforms. Sequence analysis revealed that both human and mouse FALP contain a putative transmembrane domain. Immunofluorescent analyses with FLAG epitope tagged protein transiently expressed in 3T3 L1 adipocytes and COS 7 cells shows that FALP strictly localizes at a compact membrane compartment at perinuclear region in resting states. Treatment of 3T3 L1 adipocytes with insulin induces the redistribution of FALP into numerous discrete spotty structures spreading throughout the cytoplasm. The tissue specific expression and its responsiveness to insulin suggest that FALP might be involved in a process specifically restricted to the adipose function, such as vesicular transport and protein secretion.

Polynucleotides of the invention include the following cDNAs for mouse FALP α (SEQ ID NO:9), mouse FALP β (SEQ ID NO:12), human FALP α (SEQ ID NO:15), and human FALP β (SEQ ID NO:18):

| | |
|---|---|
| Mouse FALP α cDNA: | |
| GAAAAAAAGC CACAGTCATG GCCAACGGGA CCGACGCCTC TGTCCCGCTC ACCAGCTATG AGTATTACCT | [SEQ ID NO:9] |
| GGACTACATA GACCTCATTC CTGTGGACGA GAAGAAGCTG AAAGCCAACA AGCATTCCAT TGTCATCGCC | |
| CTGTGGTTGA GCCTGGCTAC CTTCGTGGTG CTCCTCTTTC TCATCCTGCT CTACATGTCC TGGTCGGGCT | |
| OCOCACAGAT GAGGCACAGT CCCCAACCCC AGCCAATATG TTCATGGACT CACAGCTTCA ACCTCCCTCT | |
| GTGCCTCCGG AGGGCCTCCC TGCAGACAAC AGAGGAGCCA GGAAGGAGAG CTGGCACTGA CCAGTGGTTA | |
| ACGCAGCAGA GTCCTTCTGC CTCAGCCCCG GGGCCCCTGG CTCTCCCCTA GGACCAGGTC CAGGATGGAG | |
| GTCCCAGGGC ATCAGCTGGC CTCACACTCA AGCAGTGGTG AGCCTGGAGA CAGAGCGTCT CPACTGTAGA | |
| ACGGATGATG CCAGAGAGCC AGTCGGGCTC AAGCAAACGG TGAACTCCAA CCAACCCGGG CAGCTACGTC | |
| TTTTTTAGGG CCGTTTACAA TGGCCTTGAA TATAGCAGGA AACTGACCGG GACAAAACCA AGTTTACAAA | |
| GAGGACCATC ACACACATTG ATAGTGCAGC TAGGATGCAG GAGCTGCCCT GGACACAGCT GTCTCTGTTG | |
| AGCAAGCTTA GCCTGCTTGC TGCTTACATT TGCTTTGGGG GTACACAGGA AAATAAAATG TGAATTAGGA | |
| TAAAAAAAAA AAA | |
| Mouse FALP β cDNA: | |
| ATGGCCAACG GGACCGACGC CTCTGTCCCG CTCACCAGCT ATGAGTATTA CCTGGACTAC ATAGACCTCA | [SEQ ID NO:12] |
| TTCCTGTGGA CGAGAACAAC CTGAAAGCCA ACAAGCGTAA GTCGGAACAC AGGAAGGTGA CCAGGCAGAG | |
| GCTGGGGCTG GGGCTGAGCC GGGGGTGAGG CCCTCTTGGC TTTGCCCGTC CCTTACTAAC TCTGGCAGAC | |
| ATCCTCAGCA GGGTTAACAT GGCTGGTGCT CACACAGACA TAAGCGAATC AGGCATGGGA CTCAAACTGT | |
| ATCTGTAACT TTCAGTAGTA AACTTTCTCT GAGTTAACTT GCCAAAAAAA AAAAAAAAAA A | |
| Human FALP α cDNA: | |
| ATGGCCAACG GGACCAACGC CTCTGCCCCA TACTACAGCT ATGAATACTA CCTGGACTAT CTGGACCTCA | [SEQ ID NO:15] |
| TTCCCGTGGA CGAGAAGAAG CTGAAAGCCC ACAAACATTC CATCGTGATC GCATTCTGGG TGAGCCTGGC | |
| TGCCTTCGTG GTGCTGCTCT TCCTCATCTT GCTCTACATG TCCTGGTCCG CCTCCCCGCA GATGAGGAGG | |
| AACAGCCCCA AGCACCACCA AACATGCCCC TGGAGTCACG GCCTCAACCT CCACCTCTGC ATCCAGAAGT | |
| GCCTGCCGTG CCACAGGGAA CCCCTGGCAA CCTCACAGGC TCAGGCGAGC TCAGTGGAGC CAGGGAGCAG | |
| AACTGGCCCT GACCAGCCGC TACGACAGGA GAGCTCCTCC ACCTTGCCCC TCGGGGGTTT CCAGACCCAC | |
| CCCACTCTCC TCTGGGAACT GACCCTCAAT GGGGGTCCCC TCGTCAGGAG CAAGCCCAGC GAGCCTCCCC | |
| CTGGAGACAG GACCTCTCAA TTGCAGAGCT GATGTCAGTA AXFCGTGGCC ATACCTGAGT GAACTGGTGA | |
| AATCAAGCCA ACCTGGACAC ATACGTTCCT CGTTCTTCTT AGAGGCCATT TGCATGTAGC AGAAAGGGCA | |
| CCTAGGTCAA GTGCAACTAG AGCAGGAGCA TCCTATGCCT TTGACAAAGA TTGCAGTGGC CCCTCGAGTG | |
| CAGAGGTCAT CCCAGGTGTT GCTGAGTTTA TTGAGCACAC CTAGCCTGCT TGCTTACTGC TTATATTTGC | |
| TCAGGGAAGA GTAGGAAAAT AAAATATATG CAAATCAAGA GGAAAAAAAA AAAAAAAAAA AAAAAAAAA | |
| Human FALP β cDNA: | |
| ATGGCCAACG GGACCAACGC CTCTGCCCCA TACTACAGCT ATGAATACTA CCTGGACTAT | [SEQ ID NO:18] |
| CTGGACCTCA TTCCCGTGGA CGAGAAGAAG CTGAAAGCCC ACAAACATTC CATCGTGATC | |
| GCATTCTGGG TGAGCCTGGC TGCCTTCGTG GTGCTGCTCT TCCTCATCTT GCTCTACATG | |
| TCCTGGTCCG CCTCCCCGCA GATGAGCTTT AACACAGATG AATCTCTTCT GCATTCAGAA | |
| GTGCTGCCTC AAACTCGAGC TATTTCCTGT GATGAGCTCC AAGCCCCTAG AGAGGAAGGG | |
| GCGGCCTGAC GAGGCACTGG ATGGGCCTCA TGGCCTAGAA GTCCCCACAT TCCTTGCAAC | |
| TCTGATGCTG GGCACCTACA GGCCCGAGTT TATCATTTAC TGGGCAGTTC AATAAAGTCC | |
| TCTCAACTTT TCTTGTCAAA AAAAAAAAAA AAAAAAAAAA AAAAAAAAA | |

Polynucleotides of the invention also include the following coding sequences for mature mouse FALP a (SEQ ID NO:10), mature mouse FALP p (SEQ ID NO:13), mature human FALP a (SEQ ID NO:16), and mature human FALP β (SEQ ID NO:19):

| | |
|---|---|
| Mature mouse FALP α: | |
| ATG GCCAACGGGA CCGACGCCTC TGTCCCGCTC ACCAGCTATG AGTATTACCT | [SEQ ID NO:10] |
| GGACTACATA GACCTCATTC CTGTGGACGA GAAGAAGCTG AAAGCCAACA AGCATTCCAT TGTCATCGCC | |
| CTGTGGTTGA GCCTGGCTAC CTTCGTGGTG CTCCTCTTTC TCATCCTGCT CTACATCTCC TGGTCGGGCT | |
| CCCCACAGAT GAGGCACAGT CCCCAACCCC AGCCAATATG TTCATGGACT CACAGCTTCA ACCTCCCTCT | |
| GTGCCTCCGG AGGGCCTCCC TGCAGACAAC AGAGGAGCCA GGAAGGAGAG CTGGCACTGA CCAGTGGTTA | |
| ACGCAGCAGA GTCCTTCTGC CTCAGCCCCG GGGCCCCTGG CTCTCCCCTA G | |
| Mature mouse FALP β: | |
| ATGGCCAACG GGACCGACGC CTCTGTCCCG CTCACCAGCT ATGAGTATTA CCTGGACTAC ATAGACCTCA | [SEQ ID NO:13] |
| TTCCTGTGGA CGAGAAGAAG CTGAAAGCCA ACAAGCGTAA GTCGGAACAC AGGAAGGTGA CCAGGCAGAG | |
| GCTGGGGCTG GGGCTGAGCC GGGGGTGA | |
| Mature human FALP α: | |
| ATGGCCAACG GGACCAACGC CTCTGCCCCA TACTACAGCT ATGAATACTA CCTGGACTAT CTGGACCTCA | [SEQ ID NO:16] |
| TTCCCGTGGA CGAGAAGAAG CTGAAAGCCC ACAAACATTC CATCGTGATC GCATTCTGGG TGAGCCTGGC | |
| TGCCTTCGTG GTGCTGCTCT TCCTCATCTT GCTCTACATG TCCTGGTCCG CCTCCCCGCA GATGAGGAGG | |
| AACAGCCCCA AGOACCACCA AACATGCCCC TGGAGTCACG GCCTCAACCT CCACCTCTGC ATCCAGAAGT | |
| GCCTGCCGTG CCACAGGGAA CCCCTGGCAA CCTCACAGGC TCAGGCGAGC TCAGTGGAGC CAGGGAGCAG | |
| AACTGGCCCT GACCAGCCGC TACGACAGGA GAGCTCCTCC ACCTTGCCCC TCGGGGGTTT CCAGACCCAC | |
| CCCACTCTCC TCTGGGAACT GACCCTCAAT GGGGGTCCCC TCGTCAGGAG CAAGCCCAGC GAGCCTCCCC | |
| CTGGAGACAG GACCTCTCAA TTGCAGAGCT GA | |
| Mature human FALP β: | |
| ATGGCCAACG GGACCAACGC CTCTGCCCCA TACTACAGCT ATGAATACTA CCTGGACTAT CTGGACCTCA | [SEQ ID NO:19] |
| TTCCCGTGGA CGAGAAGAAG CTGAAAGCCC ACAAACATTC CATCGTGATC GCATTCTGGG TGAGCCTGGC | |
| TGCCTTCGTG GTGCTGCTCT TCCTCATCTT GCTCTACATG TCCTGGTCCG CCTCCCCGCA GATGAGCTTT | |
| AACACAGATG AATCTCTTCT GCATTCAGAA GTGCTGCCTC AAACTCGAGC TATTTCCTGT GATGAGCTCC | |
| AAGCCCCTAG AGAGGAAGGG GCGGCCTGA | |

Polypeptides of the invention include the following mature mouse FALP a (SEQ ID NO:11), mature mouse FALP β (SEQ ID NO:14), mature human FALP α (SEQ ID NO:17), and mature human FALP β (SEQ ID NO:20) amino acid sequences:

| | |
|---|---|
| Mouse FALP α: | |
| MANGTDASVP LTSYEYYLDY IDLIPVDEKK LKANKHSIVI ALWLSLATFV VLLFLILLYM SWSGSPQMRH | [SEQ ID NO:11] |
| SPQPQPICSW THSFNLPLCL RRASLQTTEE PGRRAGTDQW LTOQSPSASA PGPLALP | |
| Mouse FALP β: | |
| MANGTDASVP LTSYEYYLDY IDLIPVDEKK LKANKRKSEH RKVTRQRLGL GLSRG | [SEQ ID NO:14] |
| Human FALP α: | |
| MANGTNASAP YYSYEYYLDY LDLIPVDEKK LKAHKHSIVI AFWVSLAAFV VLLFLILLYM SWSASPQMRR | [SEQ ID NO:17] |
| NSPKHHQTCP WSHGLNLHLC IQKCLPCHRE PLATSQAQAS SVEPGSRTGP OQPLRQESSS TLPLGGFQTH | |
| PTLLWELTLN GGPLVRSKPS EPPPGDRTSQ LQS | |
| Human FALP β: | |
| MANGTNASAP YYSYEYYLDY LDLIPVDEKK LKAHKHSIVI AFWVSLAAFV VLLFLILLYM SWSASPQMSF | [SEQ ID NO:20] |
| NTDESLLHSE VLPQTRAISC DELQAPREEG AA | |

Other polypeptides of the invention include, for example, mature mouse FALP α, mature mouse FALP β, mature human FALP α, and mature human FALP β amino acid sequences that do not include an N-terminal methionine. Still other polypeptides of the invention include, for example, mature mouse FALP α, mature mouse FALP β, mature human FALP α, and mature human FALP β amino acid sequences that are C-terminally amidated. Other polypeptides of the invention include, for example, mature mouse FALP α, mature mouse FALP β, mature human FALP α, and mature human FALP β amino acid sequences that do not include an N-terminal methionine and are C-terminally amidated.

The practice of the present invention will employ, unless otherwise indicated, various conventional techniques of molecular biology (including recombinant techniques), microbiology, cell biology, biochemistry, nucleic acid chemistry, and immunology, which are within the skill of the art. Such techniques are explained fully in the literature, such as, MOLECULAR CLONING: A LABORATORY MANUAL, second edition (Sambrook et al., 1989) and MOLECULAR CLONING: A LABORATORY MANUAL, third edition (Sambrook and Russel, 2001), (jointly and individually referred to herein as “Sambrook”). OLIGONUCLEOTIDE SYNTHESIS (M. J. Gait, ed., 1984); ANIMAL CELL CULTURE (R. I. Freshney, ed., 1987); HANDBOOK OF EXPERIMENTAL IMMUNOLOGY (D. M. Weir & C. C. Blackwell, eds.); GENE TRANSFER VECTORS FOR MAMMALIAN CELLS (J. M. Miller & M. P. Calos, eds., 1987); CURRENT PROTOCOLS IN MOLECULAR BIOLOGY (F. M. Ausubel et al., eds., 1987, including supplements through 2001); PCR: THE POLYMERASE CHAIN REACTION, (Mulis et al., eds., 1994); CURRENT PROTOCOLS IN IMMUNOLOGY (J. E. Coligan et al., eds., 1991); THE IMMUNOASSAY HANDBOOK (D. Wild, ed., Stockton Press NY, 1994); BIOCONJUGATE TECHNIQUES (Greg T. Hermanson, ed., Academic Press, 1996); METHODS OF IMMUNOLOGICAL ANALYSIS (R. Masseycff, W. H. Albert, and N. A. Staines, eds., Weinheim: VCH Verlags gesellschaft mbH, 1993), Harlow and Lane (1988) ANTIBODIES, A LABORATORY MANUAL, Cold Spring Harbor Publications, New York, and Harlow and Lane (1999) USING ANTIBODIES: A LABORATORY MANUAL Cold Spring Harbor Laboratory Press, Cold Spring Harbor, N.Y. (jointly and individually referred to herein as Harlow and Lane), Beaucage et al. eds., CURRENT PROTOCOLS IN NUCLEIC ACID CHEMISTRY John Wiley & Sons, Inc., New York, 2000); and Agrawal, ed., PROTOCOLS FOR OLIGONUCLEOTIDES AND ANALOGS, SYNTHESIS AND PROPERTIES Humana Press Inc., New Jersey, 1993).

In one aspect, the invention provides a polynucleotide having a sequence or subsequence of a mammalian (for example, mouse or human) FALP gene or RNA. Other mammalian FALP polynucleotides that hybridize with a human or mouse FALP probe under stringent conditions also form a part of the invention. The polynucleotides of the invention (for example, RNA, DNA, PNA or chimeras), may be single-stranded, double stranded, or a mixed hybrid. In one aspect, the polynucleotide has a sequence of a FALP shown herein or is a subsequence thereof (for example, comprising at least 15, at least 25, at least 50, at least 100, at least 200, or at least 500 bases of the polynucleotides and variants of the invention). The invention also provides polynucleotides with substantial sequence identity to the FALP polynucleotides disclosed herein. Thus, the invention provides naturally occurring alleles of mammalian (for example, human) FALP genes such as human allelic variants of the FALP polynucleotides disclosed herein.

As described herein, in some aspects the polynucleotide of the invention encodes a polypeptide with substantial sequence similarity to disclosed herein or encodes a fragment of such a polypeptide (for example, a fusion protein). Also contemplated are polynucleotides that, due to the degeneracy of the genetic code, are not substantially similar to a naturally occurring nucleic acid sequence, but encode the polypeptide of disclosed herein or a fragment thereof. In other embodiments, the invention provides FALP polynucleotides that do not necessarily encode FALP polypeptide but which are useful as for example, probes, primers, antisense, triplex RNAi, or ribozyme reagents, and the like.

The invention also includes expression vectors, cell lines, and transgenic organisms comprising the FALP polynucleotides. In some embodiments, the vectors, cells, and organisms of the invention are capable of expressing the encoded FALP polypeptides.

Using the guidance of this disclosure, the FALP polynucleotides of the invention can be produced by recombinant means. See, for example, Sambrook et al., Berger and Kimmel, (1987) METHODS IN ENZYMOLOGY, Vol. 152: Guide To Molecular Cloning Techniques, San Diego: Academic Press, Inc.; Ausubel et al., CURRENT PROTOCOLS IN MOLECULAR BIOLOGY, Greene Publishing and Wiley-Interscience, New York (2001). Alternatively, FALP polynucleotides or fragments can be chemically synthesized using routine methods well known in the art (see, for example, Narang et al., 1979, Meth. Enzymol. 68: 90; Brown et al., 1979, Meth. Enzymol. 68: 109; Beaucage et al., 1981, Tetra. Lett., 22: 1859). In some embodiments, the FALP polynucleotides of the invention contain non-naturally occurring bases, for example, deoxyinosine (see, Batzer et al., 1991, Nucleic Acid Res. 19: 5081; Ohtsuka et al., 1985, J. Biol. Chem. 260: 2605-2608; Rossolini et al., 1994, Mol. Cell. Probes 8: 91-98) or modified backbone residues or linkages.

For use in the practice of the various aspects and embodiments of this invention, it is preferred to prepare and isolate at least a portion of some, many, or all of these FALP proteins. It may also be useful to prepare and isolate FALP homologues from other non-human species, and the teachings herein may readily be adapted for that purpose, if desired.

The FALPs of the present invention may be naturally purified products, products of chemical synthetic procedures, or produced by recombinant techniques from prokaryotic or eukaryotic host cells (for example, by bacterial, yeast, higher plant, insect, and mammalian cells in culture) into which appropriate polynucleotide constructs (for example, an expression vector) harboring a polynucleotide encoding one or more FALPs, fragments, analogs, variants, or derivatives have been introduced. Polypeptides of the invention may also, for example, include an initial methionine amino acid residue.

Preferably, a FALP protein will be prepared by obtaining a nucleic acid molecule encoding the desired protein, inserting this nucleic acid molecule into a suitable expression vector, inserting the vector into a compatible host cell, expressing the FALP protein in the host cell, and purifying the FALP protein. Depending upon the host employed in a recombinant production procedure, the FALPs of the present invention may be post-translationally modified (for example, glycosylated or methylated).

As will be appreciated, polynucleotides may be employed for producing FALPs by recombinant techniques. In order to recombinantly express a biologically active FALP polypeptide, the nucleotide sequences encoding the protein may be inserted into an appropriate expression vector, i.e., a vector that contains the necessary elements for transcriptional and translational control of the inserted coding sequence in a suitable host cell. These elements include regulatory sequences, such as enhancers, constitutive and inducible promoters, and 5′ and 3′ untranslated regions in the vector and in polynucleotide sequences encoding the FALP. Such elements may vary in their strength and specificity. Specific initiation signals may also be used to achieve more efficient translation of sequences encoding a FALP. Such signals include an ATG initiation codon and adjacent sequences, for example, the Kozak sequence. In cases where polynucleotides encoding a FALP and a 5′ initiation codon and upstream regulatory sequences are inserted into the appropriate expression vector, no additional transcriptional or translational control signals may be needed. However, in cases where only the FALP coding sequence, or a fragment thereof, is inserted, exogenous translational control signals including an in-frame ATG initiation codon should be provided by the vector. Exogenous translational elements and initiation codons may be of various origins, both natural and synthetic. The efficiency of expression may be enhanced by the inclusion of enhancers appropriate for the particular host cell system used.

Methods that are well known in the art may be used to construct expression vectors containing sequences encoding FALPs and appropriate transcriptional and translational control elements. These methods include in vitro recombinant DNA techniques, synthetic techniques, and in vivo genetic recombination, such as those as described below. See, for example, Sambrook, et al.; and Ausubel, et al. (1995, and periodic supplements), Current Protocols in Molecular Biology, John Wiley & Sons, New York, N.Y.

A nucleic acid molecule encoding a FALP can readily be obtained in a variety of ways, including, without limitation, chemical synthesis, cDNA or genomic library screening, expression library screening, and/or PCR amplification of cDNA. These methods and others useful for isolating such DNA are set forth, for example, by Sambrook, et al.; Ausubel, et al.; and by Berger and Kimmel (Methods in Enzymology: Guide to Molecular Cloning Techniques, vol. 152, Academic Press, Inc., San Diego, Calif. (1987)). Preferred nucleic acid molecules encoding FALPs are obtained from mammalian sequences. Most preferred nucleic acid molecules encoding FALPs are isolated from human (or other primates), rat, or mouse cells.

Chemical synthesis of a nucleic acid molecule encoding a FALP can be accomplished using methods well known in the art, such as those set forth by Engels, et al. (Angew. Chem. Intl. Ed., vol. 28: 716-734 (1989)); Caruthers, et al., Nucl. Acids. Symp. Ser. (7): 215-223 (1980)); and Horn, et al., Nucl. Acids Symp. Ser. (7): 225-232 (1980)). These methods include, inter alia, the phosphotriester, phosphoramidite, and H-phosphonate methods of nucleic acid synthesis. Typically, the nucleic acid molecule encoding the full length FALP polypeptide to be synthesized will be several hundred base pairs (bp) or nucleotides in length. Nucleic acids larger than about 100 nucleotides in length can be synthesized as several fragments, each fragment typically being up to about 100 nucleotides in length. The fragments can then be assembled into a FALP-encoding gene in a variety of ways, for example, by ligation of multiple double-stranded molecules having short single-stranded 5′ and 3′ regions to facilitate, followed by ligation to form a full length nucleic acid encoding the FALP polypeptide. Alternatively, the fragments can each be designed to contain 5′ and 3′ regions for annealing to nucleic acid fragments containing complementary sequences. Under suitable conditions, the fragments are annealed to produce a nucleic acid molecule containing both double- and single-stranded regions. The single stranded regions can then be made double-stranded in a reaction using an appropriate DNA polymerase. Chemical synthesis of nucleic acid molecules is especially preferred when introducing changes as compared to the naturally occurring sequence, for example, to introduce restriction sites, insert preference codons, to insert, delete, or change amino acids, alter glycosylation patterns, produce splice variants, and so on.

Alternatively, a nucleic acid encoding a FALP polypeptide may be obtained by screening an appropriate cDNA library (i.e., a library prepared from one or more tissue source(s) known or believed to express the polypeptide) or a genomic library (i.e., a library prepared from total genomic DNA). The source of the cDNA library is typically a tissue from any species that is believed to express the desired FALP in reasonable or desired quantities. The source of the genomic library may be any tissue or tissues from any mammalian or other species believed to harbor the gene encoding the desired FALP or a homologue of the desired FALP. The library can be screened for the presence of the FALP cDNA/gene using one or more nucleic acid probes (oligonucleotides, cDNA, or genomic DNA fragments that possess an acceptable level of sequence identity to the FALP or FALP homologue cDNA or gene to be cloned) that will hybridize selectively with FALP or FALP homologue cDNA(s) or gene(s) that is(are) present in the library. The probes typically used for such library screening usually encode a small region of the FALP nucleotide sequence from the same or a similar species as the species from which the library was prepared. Alternatively, the probes may be degenerate, as discussed herein.

When screening for full-length cDNAs, it is preferable to use libraries that have been size-selected to include larger cDNAs. In addition, random-primed libraries, which often include sequences containing the 5′ regions of genes, are preferable for situations in which an oligo d(T) library does not yield a full-length cDNA. Genomic libraries may be useful for extension of sequence into 5′ non-transcribed regulatory regions. The nucleic acid molecules encoding FALPs may be extended utilizing a partial nucleotide sequence and employing various PCR-based methods known in the art to detect upstream sequences, such as promoters and regulatory elements. For PCR-based methods, primers may be designed using any suitable software. Typically, PCR primers are generally designed to be about 22 to 30 nucleotides in length, have a GC content of about 50% or more, and to anneal to the template at temperatures of between about 68° C. to 72° C.

Library screening may be accomplished by annealing the probe to nucleic acid molecules from clones in the library under conditions of stringency that prevent non-specific or undesired binding but permit binding of those clones that have a significant or desired level of homology with the probe or primer. Typical hybridization and washing stringency conditions depend in part on the size (i.e., number of nucleotides in length) of the probe, and whether the probe is degenerate. The probability of obtaining a clone(s) is also considered in designing the hybridization reaction conditions (ie., whether a cDNA or genomic library is being screened; if it is a cDNA library, the probability that the cDNA of interest is present at a high level).

Where large nucleic acid fragments (such as cDNAs) are used as probes, typical hybridization conditions are those for example as set forth in Ausubel et al. After hybridization, the blot containing the library is washed at a suitable stringency, depending on several factors such as probe size, expected homology of probe to clone, type of library being screened, number of clones being screened, and the like. Additional examples of stringent washing solutions (which are usually low in ionic strength and are used at relatively high temperatures) are as follows. One such stringent wash is 0.015 M NaCl, 0.005 M NaCitrate and 0.1 percent SDS at 55-65° C. Another such stringent buffer is 1 mM Na₂EDTA, 40 mM NaHPO₄, pH 7.2, and 1% SDS at about 40-50° C. Another stringent wash is 0.2XSSC and 0.1% SDS at about 50-65° C.

Where oligonucleotide probes are used to screen cDNA or genomic libraries, two protocols for stringent washing conditions as follows may be used, for example. The first protocol uses 6XSSC with 0.05% sodium pyrophosphate at a temperature of between about 35 and 62° C., depending on the length of the probe. For example, 14 base probes are washed at 35-40° C., 17 base probes at 45-50° C., 20 base probes at 52-57° C., and 23 base probes at 57-63° C. The temperature can be increased 2-3° C. where the background non-specific binding appears high. A second protocol uses tetramethylammonium chloride (TMAC) for washing. One such stringent washing solution is 3 M TMAC, 50 mM Tris-HCl, pH 8.0, and 0.2% SDS. The washing temperature using this solution is a function of the length of the probe. For example, a 17 base probe is washed at about 45-50° C.

A preferred method for obtaining a nucleic acid encoding a FALP polypeptide is the polymerase chain reaction (PCR). In this method, PCR is used to amplify the desired FALP sequence from a nucleic acid (Le., poly(A)+RNA or total RNA, a cDNA, genomic DNA) that encodes the FALP. For example, when poly(A)+RNA or total RNA is the source of the FALP-encoding sequence, cDNA may first be prepared from the RNA using the enzyme reverse transcriptase. Two primers (oligonucleotides) typically complementary to two separate regions of the FALP cDNA are then added to the cDNA along with a polymerase (for example, Taq polymerase), and under suitable reaction conditions, the polymerase amplifies the cDNA region between the two primers.

Where the method of choice for preparing the nucleic acid encoding the FALP polypeptide requires the use of oligonucleotide primers or probes (for example, PCR, cDNA, or genomic library screening), the oligonucleotide sequences selected as probes or primers should generally be of adequate length and sufficiently unambiguous so as to minimize the amount of non-specific binding that will occur during library screening or PCR amplification. The actual sequence of the probes or primers is usually based on conserved or highly homologous sequences or regions from the same or a similar gene from another organism. Optionally, the probes or primers can be fully or partially degenerate, ie., contain a mixture of probes/primers, all encoding the same amino acid sequence, but using different codons to do so. An alternative to preparing degenerate probes is to place an inosine in some or all of those codon positions that vary by species. The oligonucleotide probes or primers may be prepared by chemical synthesis methods.

FALP mutant or variant sequences can also be employed in the practice of the invention. A mutant or variant sequence as used herein is a sequence that contains, for example, one or more nucleotide substitutions, deletions, additions and/or insertions as compared to the wild type sequence that result in amino acid sequence variations as compared to the wild type amino acid sequence. In some cases, naturally occurring FALP amino acid mutants or variants may exist, due to the existence of natural allelic variation, and may also be employed.

It will also be appreciated by those skilled in the art that as a result of the degeneracy of the genetic code, a multitude of polynucleotide sequences encoding a particular FALP, some bearing minimal similarity to the polynucleotide sequences of any known and naturally occurring gene, may be produced. Thus, the invention contemplates each and every possible variation of polynucleotide sequence that could be made by selecting combinations based on possible codon choices. These combinations are made in accordance with the standard triplet genetic code, and all such variations are to be considered as being specifically disclosed. As will be appreciated, it may be useful to engineer some or all of the coding sequence for a FALP to include codons found by statistical analysis to be over-represented in native genes expressed at high levels (so-called “preference codons”) in the host cell type selected for expression of the recombinant FALP. Other reasons for substantially altering a nucleotide sequence encoding a FALP without altering the encoded amino acid sequence include the production of mRNA transcripts having more desirable properties, such as a greater half-life, different secondary structure, etc., than transcripts produced from the naturally occurring sequence.

After generating a polynucleotide encoding a FALP polypeptide (or fragment or variant thereof, for example), the nucleotide sequence of the FALP-encoding nucleic acid is preferably determined to confirm that a nucleic acid molecule having the intended or desired nucleotide sequence has been generated. Methods for nucleic acid sequencing are well known and generally available in the art and may be used to practice any of the embodiments of the invention. The methods may employ such enzymes as the Klenow fragment of DNA polymerase I, SequenaseR™ (US Biochemical Corp., Cleveland, Ohio), Taq polymerase (Perkin Elmer), thermostable T7 polymerase (Amersham, Chicago, Ill.), or combinations of polymerases and proofreading exonucleases. Preferably, the process is automated with machines such as the Hamilton Micro Lab 2200 (Hamilton, Reno, Nev.), Peltier Thermal Cycler (PTC200; MJ Research, Watertown, Mass.), and the ABI Catalyst and 373 and 377 DNA Sequencers (Perkin Elmer).

Thus, in one aspect, the invention provides polynucleotides encoding FALP polypeptides such as a FALP polypeptide having one of the sequences disclosed herein, a fragment thereof, a variant thereof (for example, a conservative or allelic variant), or a FALP fusion polypeptide. In one embodiment, the polynucleotide of the invention comprises the sequence disclosed herein or a fragment thereof. In another embodiment, the polynucleotide encodes a naturally occurring FALP polypeptide or fragment, but has a sequence that differs from the human or mouse alleles shown herein.

The polynucleotides of invention are useful for expression of FALP polynucleotides (for example, sense or antisense RNAs) and polypeptides. As indicated herein, methods for recombinant expression of polynucleotides and polypeptides are well known in the art. Typically, the FALP polynucleotides of the invention are used in expression vectors for the preparation of FALP polypeptides and polynucleotides. Thus, in one embodiment, DNA encoding an FALP polypeptide of the present invention is inserted into DNA constructs capable of introduction into and expression in an in vitro host cell, such as a bacterial (for example, E. coli, Bacillus subtilus), yeast (for example, Saccharomyces), insect (for example, Spodoptera frugiperda), or mammalian cell culture systems. Examples of mammalian cell culture systems useful for expression and production of the polypeptides of the present invention include human embryonic kidney line (293; Graham et al., 1977, J. Gen. Virol. 36: 59); CHO (ATCC CCL 61 and CRL 9618); human cervical carcinoma cells (HeLa, ATCC CCL 2); and others known in the art. Useful human and nonhuman cell lines are widely available, for example, from the American Type Culture Collection (ATCC), P.O. Box 1549, Manassas, Va. 20108 (see http://www.atcc.org). The use of mammalian tissue cell culture to express polypeptides is discussed generally in Winnacker, FROM GENES To CLONES (VCH Publishers, N.Y., N.Y., 1987) and Ausubel, et al.

In some aspects, promoters from mammalian genes or from mammalian viruses are used, for example, for expression in mammalian cell lines. Suitable promoters can be constitutive, cell type-specific, stage-specific, and/or modulatable or regulatable (for example, by hormones such as glucocorticoids). Useful promoters include, but are not limited to, the metallothionein promoter, the constitutive adenovirus major late promoter, the dexamethasone-inducible MMTV promoter, the SV40 promoter, and promoter-enhancer combinations known in the art.

FALP polypeptides or fragments can also be expressed in transgenic animals (mouse, sheep, cow, etc.) and plants (tobacco, arabidopsis, etc.) using appropriate expression vectors which integrate into the host cell chromosome.

In another aspect, the invention provides oligonucleotide or polynucleotide probes and/or primers for detecting or amplifying FALP polynucleotides. In various embodiments, the polynucleotides (for example, probes and primers) comprise at least about 10 contiguous bases identical or exactly complementary to the (naturally occurring) FALP disclosed herein, usually at least about 12 bases, typically at least about 15 bases, generally at least about 18 bases and sometimes at least about 25, at least about 50, or at least about 100 bases When the FALP polynucleotides of the invention are used as probes or primers they are generally less that about 1000 bases in length; typically they contain between about 12 and about 500 contiguous nucleotides identical or exactly complementary the (naturally occurring) FALP, more often between about 12 and about 50 contiguous nucleotides, even more often between about 15 and about 25 contiguous nucleotides. In some aspects, the probes and primers are modified, for example, by adding restriction sites to the probes or primers. In other embodiments, primers or probes of the invention comprise additional sequences, such as linkers. In still some other embodiments, primers or probes of the invention are modified with detectable labels. For example, the primers and probes are chemically modified, for example, derivatized, incorporating modified nucleotide bases, or containing a ligand capable of being bound by an anti-ligand (for example, biotin).

The FALP probes and primers of the invention can be used for a number of purposes, for example, for detecting or amplifying an FALP polynucleotide in a biological sample, as discussed in detail herein. For example, provided with the guidance herein, one of skill will be able to select primer pairs that specifically amplify all or a portion of the FALP gene, mRNA, or cDNA in a sample.

The invention also contemplates the preparation of vectors for FALP expression. After cloning, or a cDNA or gene encoding a FALP polypeptide or fragment thereof has been isolated, it is typically inserted into an amplification and/or expression vector in order to increase the copy number of the gene and/or to express the polypeptide in a suitable host cell. The vector is often a commercially available vector, though “custom made” vectors may be used as well. The vector is selected to be functional in the particular host cell employed (i.e., the vector is compatible with the host cell machinery such that amplification of the FALP-encoding polynucleotide and/or expression of the FALP can occur). In preferred embodiments, expression vectors capable of expressing a FALP contain various operational elements. These “operational elements” include at least one promoter, at least one ribosome binding or entry sequence, and at least one terminator codon. Such “operational elements” may also include operators, signal sequences, and for proteins to be exported from intracellular space, and any other sequences necessary or preferred for appropriate transcription and subsequent translation of the FALP-encoding polynucleotide.

Additional embodiments of the present invention are envisioned as employing other known or currently undiscovered expression vectors that would contain one or more of the operational elements described herein. In particular, it is preferred that these vectors have some or all of the following characteristics: (1) possess a minimal number of host-organism sequences; (2) be stable in the desired host; (3) be capable of being present in a high copy number in the desired host; (4) possess a regulatable promoter; (5) have at least one DNA sequence coding for a selectable trait present on a portion of the vector separate from that where the FALP-encoding polynucleotide will be inserted; and (6) be integrated into the vector.

The FALP polypeptide or fragment thereof may be amplified/expressed in any suitable cloning and/or expression system, be it a prokaryotic, yeast, insect (baculovirus systems) and/or eukaryotic cell-based expression system or a cell-free expression system. In other words, any suitable expression vector/host system may be utilized to contain and express sequences encoding a FALP. These include microorganisms such as bacteria transformed with recombinant bacteriophage, plasmid, or cosmid DNA expression vectors; yeast transformed with yeast expression vectors; insect cell systems infected with viral expression vectors (for example, baculovirus); plant cell systems transformed with viral expression vectors (for example, cauliflower mosaic virus (CaMV) or tobacco mosaic virus (TMV)) or with bacterial expression vectors (for example, Ti or pBR322 plasmids); or animal cell systems. The invention is not limited by the host cell employed.

With regard to cell-based expression systems, selection of the host cell will depend at least in part on whether the FALP polypeptide or fragment thereof is to be glycosylated or phosphorylated. If so, a eukaryotic host cell-based expression system (for example, yeast, insect, or mammalian host cells) are preferable; yeast cells will glycosylate the polypeptide, and insect and mammalian cells can glycosylate and/or phosphorylate the polypeptide as occurs naturally in cells where the FALP polypeptide is made (i.e., “native” glycosylation and/or phosphorylation). Selection of the host cell for FALP polypeptide production may also be determined based in part the manner in which the host cell is able to “fold” the protein into its native tertiary structure (for example, proper orientation of disulfide bridges, etc.) such that biologically active protein is prepared. The FALP polypeptide may be “folded” after synthesis using appropriate chemical conditions, as discussed herein. In addition, a host cell strain may be chosen for its ability to modulate expression of the inserted sequences or to process the expressed protein in other desired ways. Such modifications of the polypeptide also include, but are not limited to, acetylation, carboxylation, lipidation, and acylation. Post-translational processing which cleaves a “prepro” form of the protein may also be used to specify protein targeting, folding, and/or activity. Different host cells that have specific cellular machinery and characteristic mechanisms for post-translational activities (e.g., CHO, HeLa, MDCK, HEK293, and W138) are available from the American Type Culture Collection (ATCC, Bethesda, Md.) and may be chosen to ensure the correct modification and processing of the foreign protein.

The host cells used may be prokaryotic cells such as gram negative or gram positive cells, for example, strains of E. coli, Bacillus, Streptomyces, Salmonella, and the like used for recombinant expression, or eukaryotic host cells such as yeast cells, insect cells, or vertebrate cells. Eukaryotic cells such as CHO (Chinese hamster ovary) cells, human kidney 293 cells, COS-7 cells; insect cells such as Sf4, Sf5, Sf9, and Sf21 and High 5 (all from the Invitrogen Company, San Diego, Calif.); and various yeast cells such as Saccharomyces (for example, S. cerevisiae) and Pichia species (for example, P. pastoris), are preferred.

Bacterial systems may be used for a variety of reasons, including the levels of expression that may be obtained and the wide range of readily available vectors and operational elements. In bacterial systems, a number of cloning and expression vectors may be selected depending upon the use intended for polynucleotide sequences encoding the particular FALP. For example, routine cloning, subcloning, and propagation of polynucleotide sequences encoding a FALP can be achieved using a multifunctional E. coli vector such as BluescriptRm (Stratagene) or pSport1™ plasmid (GIBCO BRL). These and other vectors available in the art may be also useful for in vitro transcription, dideoxy sequencing, single strand rescue with helper phage, and creation of nested deletions in the cloned sequence. When large quantities of a FALP is needed, for example, for high throughput screening, vectors which direct high level expression of the desired FALP may be used. For example, vectors containing the strong, inducible TS or T7 bacteriophage promoter may be used. Thus, in bacterial systems, a number of cloning and expression vectors may be selected depending upon the use intended for polynucleotide sequences encoding the particular FALP. For example, routine cloning, subcloning, and propagation of polynucleotide sequences encoding a FALP can be achieved using a multifunctional E. coli vector such as BluescriptR (Stratagene) or pSport1™ plasmid (GIBCO BRL). These and other vectors available in the art may be also useful for in vitro transcription, dideoxy sequencing, single strand rescue with helper phage, and creation of nested deletions in the cloned sequence. When large quantities of a FALP is needed, for example, for high throughput screening, vectors which direct high level expression of the desired FALP may be used. For example, vectors containing the strong, inducible T5 or T7 bacteriophage promoter may be used.

Yeast expression systems may also be used for production of FALPs. A number of vectors containing constitutive or inducible promoters, such as alpha factor, alcohol oxidase, and PGH, may be used in the yeast Saccharomyces cerevisiae or Pichia pastoris. In addition, such vectors can direct either the secretion or intracellular retention of expressed proteins and enable integration of foreign sequences into the host genome for stable propagation.

Maintenance of foreign DNA introduced into yeast can be effected in several ways (Botstein and Davis, The Molecular Biology of the Yeast Saccharomyces, Cold Spring Harbor Laboratory, Strathern, Jones and Broach, eds., pp. 607-636 (1982)). For example, an expression system for use with host organisms of the genus Saccharomyces harbors the anticollagenase gene on the 2 micron plasmid. The advantages of the 2 micron circle include relatively high copy number and stability when introduced into certain yeast strains. These vectors preferably incorporate a bacterial replication of origin and at least one antibiotic resistance marker to allow replication and selection in bacteria (for example, E. coli). In addition, the plasmid will preferably have 2 micron sequences and a yeast selectable marker gene (for example, the LEU2 gene).

Plant systems may also be used for expression of FALPs. Transcription of sequences encoding a desired FALP may be driven viral promoters, e.g., the 35S and 19S promoters of CaMV used alone or in combination with the omega leader sequence from TMV. Takamatsu, N., EMBO J., vol 6: 307-311 (1987). Alternatively, plant promoters such as the small subunit of RUBISCO or heat shock promoters may be used. These constructs can be introduced into plant cells by direct DNA transformation or pathogen-mediated transfection.

For long term production of recombinant proteins in mammalian systems, stable expression of the particular FALP in cell lines is preferred. For example, polynucleotides encoding the desired FALP can be transformed into cell lines using expression vectors which may contain viral origins of replication and/or endogenous expression elements and a selectable marker gene on the same or on a separate vector. Following the introduction of the vector, cells may be allowed to grow for about 1 to 2 days in enriched media before being switched to selective media The purpose of the selectable marker is to confer resistance to a selective agent, and its presence allows growth and recovery of cells that successfully express the introduced sequences. Resistant clones of stably transformed cells may be propagated using tissue culture techniques appropriate to the cell type.

As indicated herein, the vectors used in any of the eukaryotic host cells may contain a 5′ flanking sequence and other regulatory elements as well such as an enhancer(s), an origin of replication element, a transcriptional termination element, a complete intron sequence containing a donor and acceptor splice site, a signal peptide sequence, a ribosome binding site element, a polyadenylation sequence, a polylinker region for inserting the nucleic acid encoding the polypeptide to be expressed, and a selectable marker element. Vectors useful for expression of FALPs in a prokaryotic expression system require fewer elements to express an exogenous sequence, for example, a promoter, an origin of replication (for vectors intended to be replicated and propogated through multiple host cell divisions), and a transcription termination element. Selectable marker elements and other regulatory elements responsible for regulating expression of the FALP-encoding polynucleotide may also be included. Optionally, the vector may contain a “tag” sequence, i.e., an oligonucleotide sequence located at the 5′ or 3′ end of the FALP coding sequence that encodes polyHis (such as hexaHis) or another small immunogenic sequence. Preferably, this tag will be expressed along with the protein, and can serve as an affinity tag for later purification of the FALP polypeptide. Optionally, the tag can subsequently be removed from the purified FALP polypeptide by various means such as using a selected peptidase, for example.

In practice, it is possible to construct such vectors so as to allow them to be easily isolated, assembled, and interchanged. This facilitates assembly of numerous functional genes from combinations of these elements and a coding region for a FALP polypeptide, fragment, or variant. Further, many of these elements will be applicable in more than one host.

As indicated herein, a polynucleotide encoding a FALP in an expression vector is operatively linked to an appropriate expression control sequence(s) (for example, a promoter) to direct mRNA synthesis. Promoters are sequences that promote RNA polymerase binding and initiation of transcription and, to regulate the expression of a FALP-encoding polynucleotide, a promoter should be included in any expression vector. Representative examples of such promoters include those noted above, as well as retroviral LTRs or the SV40 early or late promoters, the CMV immediate early promoter, the HSV thymidine kinase promoter, the E. coli lacI, lacz, or trp promoter, the phage lambda P_{L} or P_{R} promoter, the T3 or T7 phage promoter, and other promoters known to control expression of genes in prokaryotic or eukaryotic host cells (or their viruses) selected for expression of a particular FALP for use in accordance with the invention. Other elements that regulate transcription, for example, operators, may optionally be included, provided that such elements function cooperatively with the promoter with which it is functionally associated. A well known example of an operator is the lac operator, which serves to activate transcription from an associated promoter in the presence of the chemical inducer isopropylthio-p-d-galactoside (IPTG). In the absence of IPTG, transcription is repressed. These and other examples of inducible expression can facilitate expression of a desired recombinant gene (for example, a FALP-encoding polynucleotide). For example, when the lac operator is present, transformed host cells containing the vector may be grown to a desired density prior to initiation of expression of the encoded FALP. When the desired density is reached, IPTG is added to relieve repression of the lac-associated promoter, thereby allowing the FALP to be expressed.

As noted herein, FALP 5′ flanking sequences, including naturally occurring FALP promoters, can also be prepared. The 5′ flanking sequence may have various transcription factor binding sites, and also may possess a TATA box and a CCAAT box. Such 5′ flanking sequences are characterized as naturally regulating transcription in vivo, either alone or in combination with other factors such a enhancer elements, repressors, and the like (any or all of which may be very distally located). Preferred 5′ flanking sequences are mammalian FALP 5′ flanking sequences. Most preferred are human FALP 5′ flanking sequences.

The 5′ flanking sequences may be obtained from genomic libraries by screening the library with cDNAs or genomic FALP fragments that preferably hybridize to the 5′ portion of a desired FALP gene. Such fragments may hybridize to a clone in the library that contains some or all of a FALP 5′ flanking sequence, which is generally located just 5′ to the start of the coding sequence for the FALP. Where the identified clone contains only a portion of the promoter, the clone itself, or a fragment of it, may be used for subsequent rounds of genomic library screening to obtain additional 5′ flanking sequence. Screening with the fragments (including hybridization and washing) may be accomplished as described above for cloning a FALP gene and/or cDNA The 5′ flanking sequence maybe homologous (i.e., from the same species and/or strain as the host cell), heterologous (i.e., from a species other than the host cell species or strain), hybrid (i.e., a combination of 5′ flanking sequences from more than one source), synthetic, or it may be the native FALP 5′ flanking sequence. As such, the source of the 5′ flanking sequence may be any unicellular prokaryotic or eukaryotic organism, any vertebrate or invertebrate organism, or any plant, provided that the 5′ flanking sequence is functional in, and can be activated by, the host cell machinery.

The 5′ flanking sequences useful in the vectors of this invention may be obtained by any of several methods well known in the art. Typically, 5′ flanking sequences useful herein other than a FALP 5′ flanking sequence will have been previously identified by mapping and/or by restriction endonuclease digestion and can thus be isolated from the proper tissue source using the appropriate restriction endonucleases. In some cases, the full nucleotide sequence of the 5′ flanking sequence may be known. In such cases, the 5′ flanking sequence may be synthesized using the methods described above for nucleic acid synthesis or cloning.

Where all or only portions of the 5′ flanking sequence are known, it may be obtained using PCR and/or by screening a genomic library with suitable oligonucleotide and/or 5′ flanking sequence fragments from the same or another species. Where the 5′ flanking sequence is not known, a fragment of DNA containing the some 5′ flanking sequence may be isolated from a larger piece of DNA that may contain, for example, a coding sequence or even another gene or genes. Isolation may be accomplished by restriction endonuclease digestion using one or more carefully selected enzymes to isolate the proper DNA fragment. After digestion, the desired fragment may be isolated by any suitable method, including agarose gel purification, a QiagenR™ column, or other methods known in the art.

An origin of replication is typically a part of prokaryotic expression vectors, and aids in the amplification of the vector in a host cell. Amplification of the vector to a certain copy number can, in some cases, be important for optimal expression of a FALP polypeptide. If the vector of choice does not contain an origin of replication, one may, for example, be chemically synthesized based on a known sequence and inserted into the vector at an appropriate location. If the vector is designed to be introduced into host cells from different species (for example, in a bacterial host cell to amplify the vector, which vector will then be introduced into a eukaryotic host cell, for example, a yeast cell of a mammalian cell for ultimate expression of the encoded FALP(s)), it may be engineered to contain multiple origins of replication. As will be appreciated, the same is also true for other elements included in a vector used in practicing the invention.

A transcription termination element is typically located 3′ to the end of a FALP polypeptide coding sequence and serves to terminate transcription of the FALP polypeptide. Usually, the transcription termination element in prokaryotic cells is a G-C rich fragment followed by a poly T sequence. Other elements 3′ to the FALP-encoding polynucleotide may also optionally be included in the vector. Included among such elements are those which stabilize mRNA or enhance transcription. While such elements are easily cloned from a library or even purchased commercially as part of a vector, they can also be readily synthesized using methods for nucleic acid synthesis such as those described above.

Selectable marker genes encode proteins typcially necessary for the survival and growth of a host cell grown in a selective culture medium, and any number of selection systems may be used to recover transformed cell lines. Typical selection marker genes encode proteins that (a) confer resistance to antibiotics or other toxins, e.g., ampicillin, tetracycline, or kanamycin for prokaryotic host cells, (b) complement auxotrophic deficiencies of the cell; or (c) supply critical nutrients not available from complex media. Accordingly, under an appropriate selection challenge (e.g, the presence of ampicillin in the media of a host cell transformed with a vector expressing a functional ampicillin resistance gene), a selectable marker allows transformants to be detected, as well as preventing the growth of contaminating microorganisms (e.g., that subset of host cells present in a transformation reaction but into which a vector as described herein has not been introduced).

Preferred selectable markers in prokaryotic systems are the kanamycin resistance gene, the ampicillin resistance gene, and the tetracycline resistance gene. Preferred selectable markers in eukaryotic systems include dihydrofolate reductase, the herpes simplex virus thymidine kinase, and adenine phosphoribosyltransferase genes for use in tk or apr cells, respectively. See, for example, Wigler, et al. (1977), Cell, vol. 11: 223-232; and Lowy, et al. (1980), Cell 22: 817-823. Also, antimetabolite, antibiotic, or herbicide resistance can be used as the basis for selection. For example, dhfr confers resistance to methotrexate; neo confers resistance to the aminoglycosides neomycin and G-418; and als or pat confer resistance to chlorsulfuron and phosphinotricin acetyltransferase, respectively. See, for example, Wigler, et al. (1980), Proc. Nat. Acad. Sci. USA, vol 77: 3567-3570; Colbere-Garapin, et al. (1981), J. Mol. Biol. 150: 1-14. Any other suitable selectable gene may also be employed.

In alternative embodiments, a selectable marker imparts a phenotypic trait, for example, expression of a protein the fluoresces in a predetermined wavelength in the presence of an appropriate substrate, energy source, and other required reaction components (which vary depending on the marker being used). Selectable markers can be used not only to identify transformants, but also to quantify the amount of transient or stable protein expression attributable to a specific vector system. See, for example, Rhodes, et al. (1995), Methods Mol. Biol., vol 55: 121-131.

Although the presence or absence of marker gene expression suggests that the gene of interest is also present, it may desirable to confirm the presence and expression of the FALP-encoding gene. In addition to assaying directly for some attribute of the encoded FALP, other approaches may also be used. For example, if the sequence encoding a FALP is inserted within a marker gene sequence, transformed cells containing polynucleotides encoding the FALP can be identified by the absence of marker gene function. Alternatively, a marker gene can be placed in tandem with a polynucleotide encoding the FALP under the control of a single promoter. Expression of the marker gene in response to induction or selection usually indicates expression of the tandem gene as well.

Ribosome binding site element(s) may also be included. This element, commonly called the Shine-Dalgarno sequence (in prokaryotes) or the Kozak sequence (in eukaryotes), is typically present for translation initiation of mRNA. The element is typically located 3′ to the promoter and 5′ to the coding sequence of the polypeptide to be synthesized. The Shine-Dalgarno sequence is varied but is typically a polypurine (i.e., having a high A-G content). Any Shine-Dalgamo sequence can be employed, and, if necessary, can be readily synthesized.

Elements set forth above, as well as others useful for the expression of FALPs as disclosed or otherwise provided for herein, are well known to the skilled artisan and are described, for example, in Sambrook, et al. (Molecular Cloning: A Laboratory Manual, Cold Spring Harbor Laboratory Press, Cold Spring Harbor, N.Y. (1989)); Ausubel, et al., eds. (Current Protocols in Molecular Biology, Current Protocols Press (1994)); and Berger and Kimmel (Methods in Enzymology: Guide to Molecular Cloning Techniques, vol. 152, Academic Press, Inc., San Diego, Calif. (1987)).

For those embodiments of the invention where the FALP is to be secreted, a signal sequence is frequently present to direct the polypeptide, for example, to a specific organelle or out of the cell (e.g., into the growth medium or periplasmic space, for example) after synthesis. Typically, the signal sequence is positioned in the FALP coding region towards or at the 5′ end of the coding region. Many signal sequences are known, and any of them that function in the selected host cell can be used. Therefore, the signal sequence may be homologous or heterologous to the polynucleotide encoding the FALP, and may be homologous or heterologous to the host cell employed. Additionally, the signal sequence may be chemically synthesized, if desired. However, for purposes herein, preferred signal sequences are those that occur naturally in the host cell being used to express the FALP.

In many cases in expression systems based on higher eukaryotic cells, transcription of the FALP gene may be increased by the presence of one or more introns on the vector. The intron may be naturally occurring within the native FALP genomic sequence, especially where the transgene is a full length or a fragment of a genomic DNA sequence. Where the intron is not naturally occurring within the FALP sequence (as for most cDNAs), the intron(s) may be obtained from another source. The intron(s) may be homologous or heterologous to the FALP-encoding polynucleotide and/or to the host cell. The position of the intron with respect to the promoter and the FALP-encoding polynucleotide is important, as the intron must be transcribed to be effective. As such, where the FALP-encoding polynucleotide is a cDNA sequence, the preferred position for the intron is 3′ to the transcription start site, and 5′ to the polyA transcription termination sequence. Preferably for cDNAs, the intron will be located on one side or the other (i.e., 5′ or 3′) of the FALP-encoding polynucleotide sequence such that it does not interrupt the FALP-encoding polynucleotide. Any intron from any source, including any viral, prokaryotic, and eukaryotic (plant or animal) organisms, may be used, provided that it is compatible with the host cell(s) into which it is inserted. Also contemplated herein are synthetic introns. Optionally, more than one intron may be used in the vector.

In another example of the invention, natural, modified, or recombinant nucleic acid molecules encoding FALPs may be engineered to produce a heterologous sequence that results in translation of a fusion protein in a suitable host system. For example, a chimeric FALP protein containing a heterologous moiety that can be recognized by a commercially available antibody may facilitate the screening of peptide libraries for inhibitors of FALP activity. Heterologous protein and peptide moieties may also facilitate purification of fusion proteins using commercially available affinity matrices. Such moieties include glutathione S-transferase (GST), maltose binding protein (MBP), thioredoxin (Trx), calmodulin binding peptide (CBP), 6-His, FLAG, c-myc, and hemagglutinin (HA). GST, MBP, Trx, CBP, and 6-His enable purification of their cognate fusion proteins on immobilized glutathione, maltose, phenylarsine oxide, calmodulin, and metal-chelate resins, respectively. FLAG, c-myc, and hemagglutinin (HA) enable immunoaffinity purification of fusion proteins using commercially available monoclonal and polyclonal antibodies that specifically recognize these epitope tags. A fusion protein may also be engineered to contain a proteolytic cleavage site located between the FALP-encoding sequence and the heterologous protein sequence, so that the FALP may be cleaved away from the heterologous moiety following purification. Methods for fusion protein expression and purification are discussed in Ausubel, et al.

Where one or more of the elements set forth above are not already present in the vector to be used (or, for that matter, other elements not described above but which are known in the art and desired to be included in a particular vector), they may be individually obtained and inserted into the vector. Methods used for obtaining each of the elements are well known in the art and may be used.

The final vectors are typically constructed from starting vectors that are commercially available. Preferred vectors for practicing this invention are those that are compatible with bacterial, insect, and mammalian host cells. Such vectors include, inter alia, pCRII, pCR3, and pcDNA3.1 (Invitrogen Company, San Diego, Calif.), pBSII (Stratagene Company, La Jolla, Calif.), pETISb (Novagen, Madison, Wis.), PGEX (Pharmacia Biotech, Piscataway, N.J.), pEGFP-N2 (Clontech, Palo Alto, Calif.), pETL (BlueBacII; Invitrogen), and pFastBacDual (Gibco/BRL, Grand Island, N.Y.). Such a vector may or may not contain some of the elements to be included in the completed vector. If one or more of the desired elements is not present in the starting vector, each element may be individually obtained and inserted into the vector (alone or, in the case of two or more elements, together), for example, by cutting the vector with the appropriate restriction endonuclease(s) such that the ends of the element to be inserted and the ends of the vector are compatible for ligation. In some cases, it may be necessary to engineer the vector (for example, by site-directed mutagenesis) to provide an appropriate sequence (for example, a restriction site) for insertion of a desired element or to “blunt” the ends to be ligated together in order to obtain a satisfactory ligation. Blunting is generally accomplished by first filling in “sticky ends” using Klenow DNA polymerase or T4 DNA polymerase in the presence of all four nucleotides (see, for example, Sambrook, et al.).

A polynucleotide encoding a FALP may be inserted into a vector by a variety of procedures. In general, the polynucleotide is inserted into an appropriate restriction endonuclease site(s) by procedures well known in the art. Preferably, the polynucleotide encoding a FALP to be expressed is assembled in appropriate phase with the appropriate regulatory elements, for example, promoter, translation initiation and termination sequences, as well as other sequences, for example, leader sequence capable of directing secretion of translated protein into the periplasmic space or extracellular medium, for example. In preferred embodiments, the vector into which the polynucleotide encoding the FALP is inserted harbors in the appropriate orientation and reading frame nucleotide sequences that, when fused with a polynucleotide encoding a FALP, encode a fusion protein that includes an N-terminal identification peptide imparting desired characteristics, for example, stabilization or simplified purification of expressed recombinant product, on the expressed protein product.

In construction of vectors, it should additionally be noted that multiple copies of the FALP-encoding polynucleotide (or one or more copies of each of at least two different FALPs) and/or its attendant operational elements may be inserted into a given vector. In such embodiments, the host organism may produce greater amounts per vector of the desired FALP(s). The number of copies of the FALP-encoding polynucleotides that may be inserted into the vector is limited only by the ability of the resultant vector, due to its size, to be transferred into and replicated and transcribed in an appropriate host microorganism.

After the vector has been constructed and a FALP-encoding polynucleotide has been inserted into the proper site of the vector, the completed vector may be inserted into a suitable host cell for amplification and/or FALP expression. Transformation of the vector into the selected host cell may be accomplished using such methods as calcium chloride precipitation, electroporation, particle bombardment, microinjection, lipofection, or the DEAE-dextran method. The method selected will in part be a function of the type of host cell to be used. These methods and other suitable methods are well known in the art, and are set forth, for example, in Sambrook, et al., supra.

Host cells transformed with polynucleotides encoding a FALP may be cultured under conditions suitable for the expression and recovery of the protein from cell culture using any suitable media, and many standard medias are well known in the art. The media will usually contain all nutrients necessary for the growth and survival of the host cells. Suitable media for culturing E. coli cells are, for example, Luria Broth (LB) and/or Terrific Broth (TB). Suitable media for culturing eukaryotic cells are RPMI 1640, MEM, DMEM, all of which may be supplemented with serum and/or growth factors as required by the particular cell line being cultured. A suitable medium for insect cultures is Grace's medium supplemented with yeastolate, lactalbumin hydrolysate, and/or fetal calf serum, as necessary. In any event, the host microorganisms are cultured under conditions appropriate for the expression of the desired gene(s). These conditions are generally specific for the host cell employed, and are readily determined by one of ordinary skill in the art, in light of the published literature regarding the growth conditions for such organisms, for example, BERGEY'S MANUAL OF DETERMINATIVE BACTERIOLOGY, 8th Ed., Williams & Wilkins Company, Baltimore, Md.

As indicated herein, an antibiotic or other compound useful for selective growth of the transformed cells only may be added as a supplement to the media. The compound to be used will be dictated by a selectable marker element present on the plasmid with which the host cell was transformed. For example, where the selectable marker element is kanamycin resistance, the compound added to the culture medium will be kanamycin.

The host cell, when cultured under appropriate conditions, can synthesize a FALP polypeptide that can subsequently be collected from the culture medium (if the host cell secretes it into the medium) or directly from the host cell producing it (if it is not secreted). As discussed above, whether a protein is secreted or retained intracellularly (e.g., by inclusion of a signal sequence that directs secretion of the FALP through a prokaryotic or eukaryotic cell membrane) can be designed into the expression vector used. After collection, the FALP polypeptide can be purified using any suitable method, or combination of methods, e.g., molecular sieve chromatography, affinity chromatography, and the like.

In general, host cells that contain a nucleic acid molecule encoding a FALP and that express the FALP may be identified by a variety of procedures known to those of skill in the art. These procedures include, but are not limited to, DNA-DNA or DNA-RNA hybridizations, PCR amplification, and protein bioassay or immunoassay techniques which include membrane, solution, or chip-based technologies for the detection and/or quantification of nucleic acids or proteins.

In addition, the amount of FALP polypeptide produced in the host cell or cell-free expression system can be evaluated using standard methods. Such methods include Western blot analysis, SDS-polyacrylamide gel electrophoresis, non-denaturing gel electrophoresis, HPLC separation, immunoprecipitation, mass spectrometry, amino acid analysis, sequencing, and/or activity assays.

The polypeptides useful in accordance with the invention are preferably provided in an isolated form, and preferably are substantially purified. If the FALP polypeptide has been designed to be secreted from the host cells, the majority of polypeptide will likely be found in the cell culture medium. Polypeptides prepared in this way will typically not possess an amino terminal methionine, as it is removed during secretion from the cell. If, however, the FALP polypeptide is not secreted from the host cells, it will be present in the cytoplasm and/or nucleus (for eukaryotic, bacteria (particularly gram positive bacteria), and insect host cells) or in the periplasm (as may occur when gram negative bacteria are used as host cells) and may have an amino terminal methionine.

For an intracellular FALP, the host cells preferably are first disrupted mechanically, osmotically, or with a detergent to release the cytoplasmic contents into a buffered solution. The FALP polypeptide is then isolated from this solution.

Purification of FALP polypeptides from solution can be accomplished using a variety of techniques. For example, if the polypeptide has been synthesized such that it contains a tag such as hexahistidine or other small tag peptide (for example, FLAG (Eastman Kodak Co., New Haven, Conn.) or myc (Invitrogen, Carlsbad, Calif.)) at either its carboxyl or amino terminus, it may essentially be purified in a one-step process by passing the solution through an affinity column where the column matrix has a high affinity for the tag or for the polypeptide directly (for example, a monoclonal antibody specifically recognizing the FALP). For example, polyhistidine binds with great affinity and specificity to nickel, thus an affinity column of nickel (such as a Qiagen nickel column) can be used for purification of a FALP containing a polyhistidine tag. See, for example, Ausubel, et al., eds.

Antibodies may also be used to purify FALPs. Antibodies to FALPs may be generated using any suitable method. For the production of antibodies, various hosts, including goats, rabbits, rats, mice, humans, and others, may be immunized by injection with a FALP or with any fragment or oligopeptide thereof having immunogenic properties. Depending on the host species, various adjuvants (for example, Freund's adjuvant, mineral gels (for example, aluminum hydroxide), and surface active substances such as lysolecithin, pluronic polyols, polyanions, peptides, oil emulsions, and dinitrophenol) may be used to increase immunological response. In addition, the immuogen can be coupled to a carrier (for example, bovine serum albumin, thyroglobulin, and keyhole limpet hemocyanin) prior to immunization.

Antibodies that bind FALPs can be prepared using intact polypeptides or fragments containing small peptides of interest as the immunizing antigen. It is preferred that the FALP-derived oligopeptides, peptides, or fragments used to induce FALP-specific antibodies preferably have an amino acid sequence consisting of at least about 5 amino acid residues, more preferably, at least about 10 amino acid residues, and even more preferably, a portion of the amino acid sequence of the natural protein and which exhibits FALP activity. The polypeptide or oligopeptide used to immunize an animal (for example, a mouse, a rat, or a rabbit) can be derived from the translation of RNA, or synthesized chemically, and can be conjugated to a carrier protein if desired.

Where the FALP polypeptide has no tag and no antibodies are available, other well known procedures for purification can be used. Such procedures include ion exchange chromatography, molecular sieve chromatography, HPLC, native gel electrophoresis in combination with gel elution, and preparative isoelectric focusing. In some cases, two or more of these techniques may be combined to achieve increased levels of purification. A particularly preferred method to isolate and purify polyhistidine-tagged FALPs is affinity chromatography using a nickel column.

If it is anticipated that the FALP polypeptide will be found primarily in the periplasmic space of the bacteria or the cytoplasm of eukaryotic cells, the contents of the periplasm or cytoplasm, including inclusion bodies (bacteria) if the processed polypeptide has formed such complexes, can be extracted from the host cell using any suitable technique. For example, the host cells can be lysed to release the contents of the periplasm by French press, homogenization, and/or sonication. The homogenate can then be centrifuged.

If the FALP polypeptide has formed inclusion bodies in the cytosol or periplasm, the inclusion bodies can often bind to the inner and/or outer cellular membranes and thus will be found primarily in the pellet material after centrifugation. The pellet material can then be treated at pH extremes or with chaotropic agent such as a detergent, guanidine, guanidine derivatives, urea, or urea derivatives in the presence of a reducing agent such as dithiothreitol at alkaline pH or tris carboxyethyl phosphine at acid pH to release, break apart, and solubilize the inclusion bodies. The FALP polypeptide in its now soluble form can then be, for example, analyzed using gel electrophoresis, immunoprecipitation or the like, or be used in screening assays. If it is desired to isolate the FALP polypeptide, isolation may be accomplished using standard methods.

In some cases, the FALP polypeptide may not be biologically active upon isolation. Various methods for “refolding” or converting the polypeptide to its native tertiary structure can be used to restore biological activity. Such methods include exposing the solubilized polypeptide to a pH usually above 7 and in the presence of a particular concentration of a chaotrope. The selection of chaotrope is very similar to the choices used for inclusion body solubilization but usually at a lower concentration and is not necessarily the same chaotrope as used for the solubilization. In most cases the refolding/oxidation solution will also contain a reducing agent or the reducing agent plus its oxidized form in a specific ratio to generate a particular redox potential allowing for disulfide shuffling to occur in the formation of the protein's cysteine bridge(s). Some of the commonly used redox couples include cysteine/cystamine, glutathione (GSH)/dithiobis GSH, cupric chloride, dithiothreitol(DTT)/dithiane DTT, 2-mercaptoethanol(bME)/dithio-b(ME). In some instances a cosolvent may be necessary to increase the efficiency of the refolding. Common reagents used for this purpose include glycerol, polyethylene glycol of various molecular weights, and arginine. After refolding, the FALP polypeptide will be soluble. It can then be, for example, analyzed using gel electrophoresis, immunoprecipitation or the like, or be used in screening assays. Again, if it is desired to isolate the FALP polypeptide, isolation of the refolded protein may be accomplished using standard methods.

If FALP-containing inclusion bodies are not formed to a significant degree in the cytosol or periplasm of the host cells, the polypeptide will be found primarily in the supernatant after centrifugation of the cell homogenate, and the FALP polypeptide can be isolated from the supernatant using any suitable method, or combination of methods. For example, in those situations where it is preferable to partially or completely isolate the FALP polypeptide, purification can be accomplished using standard methods, for example, separation by electrophoresis followed by electroelution, various types of chromatography (immunoaffinity, molecular sieve, and/or ion exchange), and/or high pressure liquid chromatography.

In those situations where it is preferable to partially or completely isolate a FALP polypeptide, purification can be accomplished using standard methods well known to the skilled artisan. Such methods include separation by electrophoresis followed by electroelution, various types of chromatography (immunoaffinity, molecular sieve, and/or ion exchange), and/or high pressure liquid chromatography. In some cases, it may be preferable to use more than one of these methods for to achieve high levels purification. Other methods include, but are not limited to, hydrophobic interaction, HPLC or affinity chromatography, to achieve the desired purity. In one embodiment, FALP polypeptides are purified using immunoaffinity chromatography. For example, antibodies raised against a FALP polypeptide or immunogenic fragment thereof (for example, having a sequence or subsequence disclosed herein) are coupled to a suitable solid support and contacted with a mixture of polypeptides containing the FALP polypeptide (for example for example, a homogenate of brain tissue) under conditions conducive to the association of this polypeptide with the antibody. Once the FALP polypeptide is bound to the immobilized antibody, the solid support is washed to remove unbound material and/or nonspecifically bound polypeptides. The desired polypeptide can then be eluted from the solid support in substantially pure form by, for example, a change in pH or salt concentration of the buffer.

As those in the art will appreciate, signal peptides, tags, etc. may be cleaved from a heterologous FALP following expression. Such cleavage may occur in the recombinant host cell if the necessary cellular machinery (for example, a peptidase for cleavage of a specific signal peptide) is present. Alternatively, such sequences may be cleaved at a later stage in the isolation and purification process.

In addition to preparing and purifying FALP polypeptide using recombinant techniques, FALP polypeptides, fragments, and/or derivatives thereof may be prepared by chemical synthesis methods (such as solid phase peptide synthesis) using techniques known in the art such as those set forth by Merrifield, et al., (J. Am. Chem. Soc., vol. 85: 2149 (1963)), Houghten, et al. (Proc. Natl. Acad. Sci. USA, vol. 82: 5132 (1985)), and Stewart and Young (Solid Phase Peptide Synthesis, Pierce Chemical Co., Rockford, Ill. (1984)). Such polypeptides may be synthesized with or without a methionine on the amino terminus. Automated synthesis may be achieved using any suitable machine, for example, the ABI 431 A peptide synthesizer (Perkin Elmer). Chemically synthesized FALP polypeptides or fragments may be oxidized using methods set forth in these references to form disulfide bridges. The FALP polypeptides or fragments are expected to have biological activity comparable to FALP polypeptides produced recombinantly or purified from natural sources, and thus may be used interchangeably with recombinant or natural FALP polypeptides.

The invention also provides inhibitory polynucleotides such as antisense, triplex, ribozyme and reagents that target or hybridize to FALP polynucleotides. Thus, in another aspect, the present invention provides antisense oligonucleotides and polynucleotides that can be used to inhibit expression of the FALP gene. Some therapeutic methods of the invention, described in additional detail infra, involve the administration of an oligonucleotide that functions to inhibit or stimulate FALP activity under in vivo physiological conditions, and is relatively stable under those conditions for a period of time sufficient for a therapeutic effect. Polynucleotides can be modified to impart such stability and to facilitate targeting delivery of the oligonucleotide to the desired tissue, organ, or cell.

The antisense polynucleotides of the invention generally comprise an antisense sequence of at least about 10 bases, typically at least 12 or 14, and up to about 3000 contiguous nucleotides that specifically hybridize to a sequence from mRNA encoding FALP or mRNA transcribed from the FALP gene. More often, the antisense polynucleotide of the invention is from about 12 to about 50 nucleotides in length or from about 15 to about 25 nucleotides in length. In general, the antisense polynucleotide should be long enough to form a stable duplex but short enough, depending on the mode of delivery, to administer in vivo, if desired. The minimum length of a polynucleotide required for specific hybridization to a target sequence depends on several factors, such as G/C content, positioning of mismatched bases (if any), degree of uniqueness of the sequence as compared to the population of target polynucleotides, and chemical nature of the polynucleotide (for example, methylphosphonate backbone, peptide nucleic acid, phosphorothioate), among other factors.

Generally, to assure specific hybridization, the antisense sequence is substantially complementary to the target FALP mRNA sequence. In certain manifestations, the antisense sequence is exactly complementary to the target sequence. The antisense polynucleotides may also include, however, nucleotide substitutions, additions, deletions, transitions, transpositions, or modifications, or other nucleic acid sequences or non-nucleic acid moieties so long as specific binding to the relevant target sequence corresponding to FALP RNA or its gene is retained as a functional property of the polynucleotide.

In another aspect, the antisense sequence is complementary to relatively accessible sequences of the FALP mRNA (for example, relatively devoid of secondary structure). This can be determined by analyzing predicted RNA secondary structures using, for example, the MFOLD program (Genetics Computer Group, Madison Wis.) and testing in vitro or in vivo as is known in the art. Another useful method for identifying effective antisense compositions uses combinatorial arrays of oligonucleotides (see, for example, Milner et al., Nature Biotechnology 15: 537 (1997)).

The invention also provides an antisense polynucleotide that has sequences in addition to the antisense sequence (i.e., in addition to anti-FALP-sense sequence). In this case, the antisense sequence is contained within a polynucleotide of longer sequence. In another embodiment, the sequence of the polynucleotide consists essentially of, or is, the antisense sequence.

The antisense nucleic acids (DNA, RNA, modified, analogues, and the like) can be made using any suitable method for producing a nucleic acid, such as the chemical synthesis and recombinant methods disclosed herein. In one embodiment, for example, antisense RNA molecules of the invention may be prepared by de novo chemical synthesis or by cloning. For example, an antisense RNA that hybridizes to FALP mRNA can be made by inserting (ligating) an FALP DNA sequence in reverse orientation operably linked to a promoter in a vector (for example, plasmid). Provided that the promoter and, preferably termination and polyadenylation signals, are properly positioned, the strand of the inserted sequence corresponding to the noncoding strand will be transcribed and act as an antisense oligonucleotide of the invention. The antisense oligonucleotides of the invention can be used to inhibit FALP activity in cell-free extracts, cells, and animals, including mammals and humans.

For general methods relating to antisense polynucleotides, see ANTISENSE RNA AND DNA, D. A. Melton, Ed., Cold Spring Harbor Laboratory, Cold Spring Harbor, N.Y. (1988)). See also, Dagle et al., Nucleic Acids Research, 19: 1805 (1991). For a review of antisense therapy, see, for example, Uhlmann et al., Chem. Reviews, 90: 543-584 (1990).

The present invention also provides oligo- and polynucleotides (for example, DNA, RNA, PNA or the like) that bind to double-stranded or duplex FALP nucleic acids (for example, in a folded region of the FALP RNA or in the FALP gene), forming a triple helix-containing, or “triplex” nucleic acid. Triple helix formation results in inhibition of FALP expression by, for example, preventing transcription of the FALP gene, thus reducing or eliminating FALP activity in a cell. Without intending to be bound by any particular mechanism, it is believed that triple helix pairing compromises the ability of the double helix to opcn sufficiently for the binding of polymerases, transcription factors, or regulatory molecules to occur.

Triplex oligo- and polynucleotides of the invention are constructed using the base-pairing rules of triple helix formation (see, for example, Cheng et al., J. Biol. Chem. 263: 15110 (1988); Ferrin and Camerini-Otero, Science 354: 1494 (1991); Ramdas et al., J. Biol. Chem. 264: 17395 (1989); Strobel et al., Science 254: 1639 (1991); and Rigas et al., Proc. Natl. Acad. Sci. U.S.A. 83: 9591 (1986)) and the FALP mRNA and/or gene sequence. Typically, the triplex-forming oligonucleotides of the invention comprise a specific sequence of from about 10 to about 25 nucleotides or longer “complementary” to a specific sequence in the FALP RNA or gene (i.e., large enough to form a stable triple helix, but small enough, depending on the mode of delivery, to administer in vivo, if desired). In this context, “complementary” means able to form a stable triple helix. In one embodiment, oligonucleotides are designed to bind specifically to the regulatory regions of the FALP gene (for example, the FALP 5′-flanking sequence, promoters, and enhancers) or to the transcription initiation site, (for example, between −10 and +10 from the transcription initiation site). For a review of recent therapeutic advances using triplex DNA, see Gee et al., in Huber and Carr, 1994, MOLECULAR AND IMMUNOLOGIC APPROACHES, Futura Publishing Co, Mt Kisco NY and Rininsland et al., 1997, Proc. Natl. Acad. Sci. USA 94: 5854.

The present invention also provides ribozymes useful for inhibition of FALP activity. The ribozymes of the invention bind and specifically cleave and inactivate FALP mRNA. Useful ribozymes can comprise 5′- and 3′-terminal sequences complementary to the FALP mRNA and can be engineered by one of skill on the basis of the FALP mRNA sequence disclosed herein (see PCT publication WO 93/23572, supra). Ribozymes of the invention include those having characteristics of group I intron ribozymes (Cech, Biotechnology 13: 323 (1995)) and others of hammerhead ribozymes (Edgington, Biotechnology 10: 256 (1992)).

Ribozymes of the invention include those having cleavage sites such as GUA, GUU and GUC. Other optimum cleavage sites for ribozyme-mediated inhibition of FALP activity in accordance with the present invention include those described in PCT publications WO 94/02595 and WO 93/23569, both incorporated herein by reference. Short RNA oligonucleotides between 15 and 20 ribonucleotides in length corresponding to the region of the target FALP gene containing the cleavage site can be evaluated for secondary structural features that may render the oligonucleotide more desirable. The suitability of cleavage sites may also be evaluated by testing accessibility to hybridization with complementary oligonucleotides using ribonuclease protection assays, or by testing for in vitro ribozyme activity in accordance with standard procedures known in the art.

As described by Hu et al., PCT publication WO 94/03596, antisense and ribozyme functions can be combined in a single oligonucleotide. Moreover, ribozymes can comprise one or more modified nucleotides or modified linkages between nucleotides, as described above in conjunction with the description of illustrative antisense oligonucleotides of the invention.

In one embodiment, the ribozymes of the invention are generated in vitro and introduced into a cell or patient. In another embodiment, gene therapy methods are used for expression of ribozymes in a target cell ex vivo or in vivo.

The present invention also provides polynucleotides useful for inhibition of FALP activity by methods such as RNA interference (RNAi), which may also include cosuppression and quelling. This and other techniques of gene suppression are well known in the art. A review of this technique is found in Science 288: 1370-1372 (2000). RNAi operates on a post-transcriptional level and is sequence specific. The process comprises introduction of RNA with partial or fully double-stranded character, or precursors of or able to encode such RNA into the cell or into the extracellular environment.

As described by Fire et al., U.S. Pat. No. 6,506,559, the RNA may comprise one or more strands of polymerized ribonucleotide. The double-stranded structure may be formed by a single self-complementary RNA strand or two complementary RNA strands. The RNA may include modifications to either the phosphate-sugar backbone or the nucleosides. RNA duplex formation may be initiated either inside or outside the cell.

Studies have demonstrated that one or more ribonucleases specifically bind to and cleave double-stranded RNA into short fragments. The ribonuclease(s) remains associated with these fragments, which in turn specifically bind to complementary mRNA, i.e., specifically bind to the transcribed mRNA strand for the FALP gene. The mRNA for the FALP gene is also degraded by the ribonuclease(s) into short fragments, thereby obviating translation and expression of the FALP gene, and so inhibiting FALP activity. Additionally, an RNA polymerase may act to facilitate the synthesis of numerous copies of the short fragments, which exponentially increases the efficiency of the system. A unique feature of this gene suppression pathway is that silencing is not limited to the cells where it is initiated. The gene-silencing effects may be disseminated to other parts of an organism and even transmitted through the germ line to several generations.

Specifically, polynucleotides of the present invention are useful for generating gene constructs for silencing FALP genes. Polynucleotides of the present invention may be used to generate genetic constructs that encode a single self-complementary RNA sequence specific for FALP genes. Genetic constructs and/or FALP-specific self-complementary RNA sequences may be delivered by any conventional method known in the art. Within genetic constructs, sense and antisense sequences flank an intron sequence arranged in proper splicing orientation making use of donor and acceptor splicing sites. Alternative methods may employ spacer sequences of various lengths rather than discrete intron sequences to create an operable and efficient construct. During post-transcriptional processing of the FALP gene construct product, intron sequences are spliced-out, allowing sense and antisense sequences, as well as splice junction sequences, to bind forming double-stranded RNA. Select ribonucleases bind to and cleave the double-stranded RNA, thereby initiating the cascade of events leading to degradation of FALP mRNA gene sequences, and silencing FALP gene(s).

Alternatively, rather than using a gene construct to express the self-complementary RNA sequences, the FALP-specific double-stranded RNA segments are delivered to one or more targeted areas to be internalized into the cell cytoplasm to exert a gene silencing effect. The RNA may be introduced in an amount which allows delivery of at least one copy per cell. Higher doses of double-stranded material may yield more effective inhibition. Inhibition is sequence-specific in that nucleotide sequences corresponding to the duplex region of the RNA are targeted for genetic inhibition. RNA containing a nucleotide sequences identical to a portion of the FALP gene is preferred for inhibition. RNA sequences with insertions, deletions, and single point mutations relative to the target sequence have also been found to be effective for inhibition. Thus, sequence identity may optimized by alignment algorithms known in the art and calculating the percent difference between the nucleotide sequences. Alternatively, the duplex region of the RNA may be defined functionally as a nucleotide sequence that is capable of hybridizing with a portion of the target gene transcript.

Therapeutic methods of the invention may involve the administration of an oligonucleotide that functions to inhibit or stimulate FALP activity under in vivo physiological conditions, and is relatively stable under those conditions for a period of time sufficient for a therapeutic effect. As noted herein, modified nucleic acids may be useful in imparting such stability, as well as for targeting delivery of the oligonucleotide to the desired tissue, organ, or cell. Oligo- and poly-nucleotides can be delivered directly as a drug in a suitable pharmaceutical formulation, or indirectly by means of introducing a nucleic acid into a cell, including liposomes, immunoliposomes, ballistics, direct uptake into cells, and the like as described herein. For treatment of disease, the oligonucleotides of the invention will be administered to a patient in a therapeutically effective amount. A therapeutically effective amount is an amount sufficient to ameliorate the symptoms of the disease or modulate FALP activity in the target cell. Methods useful for delivery of oligonucleotides for therapeutic purposes are described in U.S. Pat. No. 5,272,065. Other details of administration of pharmaceutically active compounds are provided herein. In another embodiment, oligo- and poly-nucleotides can be delivered using gene therapy and recombinant DNA expression plasmids of the invention.

Gene therapy refers to the introduction of an otherwise exogenous polynucleotide which produces a medically useful phenotypic effect upon the (typically) mammalian cell(s) into which it is transferred. In one aspect, the present invention provides gene therapy methods and compositions for treatment of FALP-associated conditions. In illustrative examples, gene therapy involves introducing into a cell a vector that expresses an FALP gene product (such as an FALP protein substantially similar to the FALP polypeptide having a sequence disclosed herein, for example, to increase FALP activity, or an inhibitory FALP polypeptide to reduce activity), expresses a nucleic acid having an FALP gene or mRNA sequence (such as an antisense RNA, for example, to reduce FALP activity), expresses a polypeptide or polynucleotide that otherwise affects expression of FALP gene products (for example, a ribozyme directed to FALP mRNA to reduce FALP activity), or replaces or disrupts an endogenous FALP sequence (for example for example, gene replacement and gene knockout, respectively). Numerous other embodiments will be evident to one of skill upon review of the disclosure herein.

Vectors useful in FALP gene therapy can be viral or nonviral, and include those described herein in relation to the FALP expression systems of the invention. It will be understood by those of skill in the art that gene therapy vectors may comprise promoters and other regulatory or processing sequences, such as are described in this disclosure as well as others. Usually the vector will comprise a promoter and, optionally, an enhancer (separate from any contained within the promoter sequences) that serve to drive transcription of an oligoribonucleotide, as well as other regulatory elements that provide for episomal maintenance or chromosomal integration and for high-level transcription, if desired. A plasmid useful for gene therapy can comprise other functional elements, such as selectable markers, identification regions, and other sequences. The additional sequences can have roles in conferring stability both outside and within a cell, targeting delivery of FALP nucleotide sequences (sense or antisense) to a specified organ, tissue, or cell population, mediating entry into a cell, mediating entry into the nucleus of a cell and/or mediating integration within nuclear DNA. For example, aptamer-like DNA structures, or other protein binding moieties sites can be used to mediate binding of a vector to cell surface receptors or to serum proteins that bind to a receptor thereby increasing the efficiency of DNA transfer into the cell. Other DNA sites and structures can directly or indirectly bind to receptors in the nuclear membrane or to other proteins that go into the nucleus, thereby facilitating nuclear uptake of a vector. Other DNA sequences can directly or indirectly affect the efficiency of integration.

Suitable gene therapy vectors may, or may not, have an origin of replication. For example, it is useful to include an origin of replication in a vector for propagation of the vector prior to administration to a patient. However, the origin of replication can often be removed before administration if the vector is designed to integrate into host chromosomal DNA or bind to host mRNA or DNA.

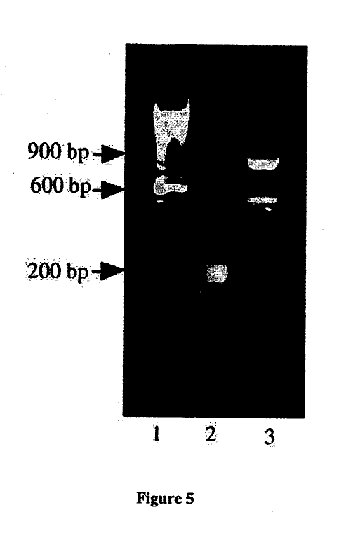

As noted, the present invention also provides methods and reagents for gene replacement therapy (i.e., replacement by homologous recombination of an endogenous FALP gene with a recombinant gene). Vectors specifically designed for integration by homologous recombination may be used. Important factors for optimizing homologous recombination include the degree of sequence identity and length of homology to chromosomal sequences. The specific sequence mediating homologous recombination is also important, because integration occurs much more easily in transcriptionally active DNA. Methods and materials for constructing homologous targeting constructs are described by for example, Mansour et al., 1988, Nature 336: 348; Bradley et al., Bio/Technology 10: 534 (1992). See also, U.S. Pat. Nos. 5,627,059; 5,487,992; 5,631,153; and 5,464,764. In one aspect, gene replacement therapy involves altering or replacing all or a portion of the regulatory sequences controlling expression of the FALP gene that is to be regulated. For example, the FALP promoter sequences (FIG. 5) may be disrupted (to decrease FALP expression or to abolish a transcriptional control site) or an exogenous promoter (for example, to increase FALP expression) substituted.

The invention also provides methods and reagents for FALP “gene knockout” (i.e., deletion or disruption by homologous recombination of an endogenous FALP gene using a recombinantly produced vector). In gene knockout, the targeted sequences can be regulatory sequences (for example, the FALP promoter), or RNA or protein coding sequences. The use of homologous recombination to alter expression of endogenous genes is described in detail in U.S. Pat. No. 5,272,071, WO 91/09955, WO 93/09222, WO 96/29411, WO 95/31560, and WO 91/12650. See also, Moynahan et al., 1996, Hum. Mol. Genet. 5.875.

Gene therapy vectors may be introduced into cells or tissues in vivo, in vitro or ex vivo. For ex vivo therapy, vectors may be introduced into cells, for example, stem cells, taken from the patient and clonally propagated for autologous transplant back into the same patient (see, for example, U.S. Pat. Nos. 5,399,493 and 5,437,994).

The invention also provides transgenic non-human multicellular organisms (for example, plants and non-human animals) or unicellular organisms (for example, yeast) comprising an exogenous FALP gene sequence, which may be a coding sequence or a regulatory (for example, promoter) sequence. Examples of multicellular organisms include plants, insects, and nonhuman animals such as mice, rats, rabbits, monkeys, apes, pigs, and other nonhuman mammals. An example of a unicellular organism is a yeast. In one example, the organism expresses an exogenous FALP polypeptide, having a sequence of a human FALP protein.

The invention also provides unicellular and multicellular organisms (or cells therefrom) in which a gene encoding FALP is mutated or deleted (i.e., in a coding or regulatory region) such that native FALP is not expressed, or is expressed at reduced levels or with different activities when compared to wild-type cells or organisms. Such cells and organisms are often referred to as “gene knock-out” cells or organisms.

The invention further provides cells and organisms in which an endogenous FALP gene is either present or optionally mutated or deleted and an exogenous FALP gene or variant (for example, human FALP) is introduced and expressed. Cells and organisms of this type will be useful, for example, as model systems for identifying modulators of FALP activity or expression; determining the effects of mutations in the FALP gene.

Methods for alteration or disruption of specific genes (for example, endogenous FALP genes) are well known to those of skill, see, for example, Baudin et al., Nucl. Acids Res. 21: 3329 (1993); Wach et al., Yeast 10: 1793 (1994); Rothstein, Methods Enzymol. 194: 281 (1991); Anderson, Methods Cell Biol. 48: 31 (1995); Pettitt et al., Development 122: 4149-4157 (1996); Ramirez-Solis et al., Methods Enzymol. 225: 855 (1993); and Thomas et al., Cell 51: 503 (1987). Typically, such methods involve altering or replacing all or a portion of the regulatory sequences controlling expression of the particular gene to be regulated. The regulatory sequences, for example, the native promoter can be altered. One conventional technique for targeted mutation of genes involves placing a genomic DNA fragment containing the gene of interest into a vector, followed by cloning of the two genomic arms associated with the targeted gene around a selectable heomycin-resistance cassette in a vector containing thymidine kinase. This “knock-out” construct is then transfected into the appropriate host cell, i.e., a mouse embryonic stem (ES) cell, which is subsequently subjected to positive selection (using G418, for example, to select for neomycin-resistance) and negative selection (using, for example, FIAU to exclude cells lacking thymidine kinase), allowing the selection of cells which have undergone homologous recombination with the knockout vector. This approach leads to inactivation of the gene of interest. See, for example, U.S. Pat. Nos. 5,464,764; 5,631,153; 5,487,992; and, 5,627,059. “Knocking out” expression of an endogenous gene can also be accomplished by the use of homologous recombination to introduce a heterologous nucleic acid into the regulatory sequences (for example, promoter) of the gene of interest. To prevent expression of functional enzyme or product, simple mutations that either alter the reading frame or disrupt the promoter can be suitable. To up-regulate expression, a native promoter can be substituted with a heterologous promoter that induces higher levels of transcription. Also, “gene trap insertion” can be used to disrupt a host gene, and mouse ES cells can be used to produce knockout transgenic animals, as described for example, in Holzschu (1997) Transgenic Res 6: 97-106. Other methods are known in the art.

Altering the expression of endogenous genes by homologous recombination can also be accomplished by using nucleic acid sequences comprising the structural gene in question. Upstream sequences are utilized for targeting heterologous recombination constructs. Utilizing FALP structural gene sequence information, such as SEQ ID NO:1, one of skill in the art can create homologous recombination constructs with only routine experimentation. Homologous recombination to alter expression of endogenous genes is described in U.S. Pat. No. 5,272,071, and WO 91/09955, WO 93/09222, WO 96/29411, WO 95/31560, and WO 91/12650. Homologous recombination in animals has been described by Moynahan (1996) Hum. Mol. Genet. 5: 875, and in plants by Offringa (1990) EMBO J. 9: 3077.

As noted, the present invention also provides isolated, substantially pure, synthetic or recombinant FALP polypeptides and immunogenic fragments of mammalian FALP polypeptides. In one embodiment, the FAUP polypeptide or fragment has an amino acid sequence identical to, or substantially identical to, a sequence set forth herein or a subsequence thereof. In other embodiments, the FALP polypeptides are variants and mutants characterized by conservative substitutions of amino acid residues disclosed herein. The polypeptides of the invention may be full-length or may encode a fragment of the full-length protein (for example, comprising at least 20, at least 40, at least 60 or at least 100 residues of the FALP polypeptides and variants of the invention. Also provided by the invention are FALP polypeptides that are modified, relative to the amino acid sequence disclosed herein, in some manner, for example, truncated, mutated, derivatized, or fused to other sequences (for example, to form a fusion protein). Some FALP polypeptides comprise insertions, deletions or substitutions of amino acid residues. For example, some conservative amino acid substitutions can be made, i.e., substitution of selected amino acids with different amino acids having similar structural characteristics, for example, net charge, hydrophobicity, and the like. Typically, the FALP variants are structurally and functionally similar to one or more of the FALP alleles shown in the Figures and/or herein. Structural similarity is indicated by, for example, substantial sequence identity (as defined above), and/or immunological cross-reactivity. As indicated herein, FALP polypeptides of the present invention can be prepared using recombinant or synthetic methods, or can be isolated from natural cellular sources.

In some embodiments, the FALP polypeptide of the invention may be used as an immunogen (for example, to produce anti-FALP antibodies). Typically, the immunogenic FALP fragments of the invention comprise at least about 6 contiguous residues disclosed herein, more often at least about 8, about 10, or about 12, or about 16 contiguous residues.

The substantially pure, isolated or recombinant FALP polypeptides of the present invention can also be characterized by their ability to bind antibodies that are specifically immunoreactive with a polypeptide having the sequence shown in the Figures. Specific immunoreactivity is usually characterized by a specific binding affinity of an antibody for its ligand (for example, FALP) of at least about 10⁷, 10⁸, 10⁹, or 10¹⁰ M⁻¹.

For various applications, it will be desirable to provide FALP polypeptides of the invention as labeled entities, i.e., covalently attached or linked to a detectable label or group, or cross-linkable group, to facilitate identification, detection and quantification of the polypeptide in a given circumstance. These detectable groups can comprise a detectable polypeptide group, for example, an assayable enzyme or antibody epitope. Alternatively, the detectable group can be selected from a variety of other detectable groups or labels, such as radiolabels (for example, ¹²⁵I, ³²P, ³⁵S) or a chemiluminescent or fluorescent group. Similarly, the detectable group can be a substrate, cofactor, inhibitor or affinity ligand.

In addition, for example, a FALP polypeptide can be modified by substituting one or more amino acid residues with a D-amino acid of the same type (for example, D-lysine in place of L-lysine) to generate more stable peptides. Similarly, modification of the amino or carboxyl terminals can also be used to confer stabilizing properties upon the polypeptides of the invention, for example, amidation of the carboxyl-terminus or acylation of the amino-terminus or pegylated derivatives.

Although primarily described in terms of “proteins” or “polypeptides,” one of skill in the art will understand that structural analogs and derivatives of the above-described polypeptides, for example, peptidomimetics, and the like can be used as substitutes for FALP, for example, as FALP agonists, or, alternatively, as FALP activity antagonists. Peptidomimetics, or peptide mimetics, are peptide analogs commonly used in the pharmaceutical industry as non-peptide drugs with properties (for example, a biological activity) analogous to those of the template peptide (Fauchere, Adv. Drug Res. 15: 29 (1986); Evans et al., J. Med. Chem. 30: 1229 (1987)). They may be developed with the aid of computerized molecular modeling. Peptide mimetics that are structurally similar to therapeutically useful peptides can be used to produce an equivalent therapeutic effect. Peptide mimetics can have significant advantages over polypeptide embodiments, including, for example, more economical production and greater chemical stability.

The present invention also provides antibodies that are specifically immunoreactive with human FALP polypeptide. Accordingly, the antibodies of the invention will specifically recognize and bind polypeptides which have an amino acid sequence identical, or substantially identical, to the amino acid sequence disclosed herein, or an immunogenic fragment thereof. The antibodies of the invention usually exhibit a specific binding affinity for FALP of at least about 10⁷, 10⁸, 10⁹, or 10¹¹ M⁻¹. The anti-FALP antibodies of the invention have a variety of uses, for example for example, isolation of FALP polypeptides (for example, by immunoaffinity chromatography), detection of FALP polypeptides, and for inhibition of FALP activity (for example, in vivo or in vitro).

Anti-FALP antibodies of the present invention can be made by a variety of means well known to those of skill in the art, for example, as described herein. Antibodies include fragments, chimeras and similar binding agents (for example, the products of phage display technology), that specifically binds an FALP polypeptide or epitope. In one aspect, the antibody is a recombinantly prepared double-chain polypeptides containing antibody light and heavy chain variable domains sufficient for antigen-specific binding, and at least a fragment of antibody light and heavy chain constant regions (for example, the CH1 domain of the heavy chain) sufficient to maintain association of the two polypeptides. In one aspect, the antibody is a single chain antibody (sFv), for example comprising antibody light and heavy chain variable domains configured to bind FALP. The invention also contemplates intrabodies against a FALP.

Methods for production of polyclonal or monoclonal antibodies are well known in the art. See, for example, Coligan, CURRENT PROTOCOLS IN IMMUNOLOGY, Wiley/Greene, NY (1991); Stites et al. (eds.) BASIC AND CLINICAL IMMUNOLOGY (7th ed.) Lange Medical Publications, Los Altos, Calif., and references cited therein (“Stites”); Goding, MONOCLONAL ANTIBODIES: PRINCIPLES AND PRACTICE (2d ed.) Academic Press, New York, N.Y. (1986); Kohler and Milstein, 1975, Nature 256: 495-97; and Harlow and Lane. These techniques include antibody preparation by selection of antibodies from libraries of recombinant antibodies in phage or similar vectors. See, Huse et al., 1989, Science 246: 1275-81; and Ward et al., 1989, Nature 341: 544-46. For production of polyclonal antibodies, an appropriate target immune system is selected, typically a mouse or rabbit, but also including goats, sheep, cows, chickens, guinea pigs, monkeys and rats. The immunoglobulins produced by the host can be precipitated, isolated and purified by routine methods, including affinity purification. Substantially monospecific antibody populations can be produced by chromatographic purification of polyclonal sera For monoclonal antibodies, appropriate animals will be selected and the desired immunization protocol followed.

The antibodies of the invention may be of any isotype, for example, IgM, IgD, IgG, IgA, and IgE, with IgG, IgA and IgM most referred. Preferred monoclonal anti-FALP antibodies neutralize (i.e., inhibit or block) one or more biological activities of FALP. Such antibodies may be obtained by screening hybridoma supernatants for the desired inhibitory activity. Monoclonal antibodies with affinities of about 10⁸ liters/mole, preferably about 10⁹ to about 10¹⁰ or stronger, can be produced by the methods described below. The production of non-human monoclonal antibodies, for example, murine, lagomorpha, or equine, is well known and can be accomplished by, for example, immunizing a host animal with a preparation containing FALP or fragments thereof. Antibody-producing cells obtained from the immunized animals are immortalized and screened, or screened first for the production of antibody which binds to the FALP polypeptide and then immortalized.

Some anti-FALP monoclonal antibodies of the present invention are humanized, human or chimeric, in order to reduce their potential antigenicity, without reducing their affinity for their target. Humanized antibodies have been described in the art. See, for example, Queen, et al., Proc. Nat'l Acad. Sci. USA 86: 10029 (1989); U.S. Pat. Nos. 5,563,762; 5,693,761; 5,585,089 and 5,530,101. The human antibody sequences used for humanization can be the sequences of naturally occurring human antibodies or can be consensus sequences of several human antibodies. See Kettleborough et al., Protein Engineering 4: 773 (1991); Kolbinger et al., Protein Engineering 6: 971 (1993). Humanized monoclonal antibodies against FALP can also be produced using transgenic animals having elements of a human immune system (see, for example, U.S. Pat. Nos. 5,569,825; 5,545,806; 5,693,762; 5,693,761; and 5,7124,350).

Useful anti-FALP antibodies can also be produced using phage display technology (see, for example, Dower et al., WO 91/17271 and McCafferty et al., WO 92/01047). In these methods, libraries of phage are produced in which members display different antibodies on their outer surfaces. Antibodies are usually displayed as Fv or Fab fragments. Phage displaying antibodies with a desired specificity are selected by affinity enrichment to an FALP polypeptide. Single chain antibodies can be producted using methods well known in the art (see, for example, Colcher et al., Ann. NY Acad. Sci. 880: 263-80 (1999); Reiter, Clin. Cancer Res. 2: 245-52 (1996); U.S. Pat. Nos. 4,946,778; 5,260,203; 5,455,030; 5,518,889; and 5,534,621).

Once expressed, the whole antibodies, their dimers, individual light and heavy chains, or other immunoglobulin forms of the present invention can be purified according to standard procedures of the art, including ammonium sulfate precipitation, affinity chromatography, gel electrophoresis and the like (see generally PROTEIN PURIFICATION: PRINCIPLES AND PRACTICE 3RD EDITION (Springer-Verlag, N.Y., 1994)).

An antibody (for example an anti-FALP antibody), is substantially pure when at least about 80%, and can be at least about 901/o, at least about 95%, or at least about 99% or more of the polypeptide molecules present in a preparation specifically bind the same antigen (for example, FALP polypeptide). For pharmaceutical uses, anti-FALP immunoglobulins of at least about 90 to 95% homogeneity are preferred, and 98 to 99% or more homogeneity are most preferred.

The antibodies of the present invention can be used with or without modification. Frequently, the antibodies will be labeled by joining, either covalently or non-covalently, a substance which provides for a detectable signal. Such labels include those that are well known in the art, for example, radioactive, fluorescent, or bioactive (for example, enzymatic) labels. As labeled binding entities, the antibodies of the invention may be particularly useful in diagnostic applications.

Also encompassed by the invention are hybrid antibodies that share the specificity of antibodies against a FALP polypeptide but are also capable of specific binding to a second moiety. In hybrid antibodies, one heavy and light chain pair is from one antibody and the other pair from an antibody raised against another epitope. This results in the property of multi-functional valency, i.e., ability to bind at least two different epitopes simultaneously. Such hybrids can be formed by fusion of hybridomas producing the respective component antibodies, or by recombinant techniques.

In one aspect of the invention, expression of FALP is monitored or determined for diagnosis of an individual with a disease state or a propensity toward a disease state, disorder or condition associated with FALP regulation. In various embodiments, the disease state is obesity or an obesity-related condition.

The invention also provides assay methods which are capable of screening compounds that modulate the activity of a FALP or for the ability modulate the activity of a FALP. This invention is particularly useful for screening compounds by using recombinant FALP in a variety of drug screening techniques. Thus, the present invention includes methods to evaluate putative specific agonists or antagonists of FALP function. Accordingly, the present invention is directed to the use of these compounds in the preparation and execution of screening assays for compounds which modulate the activity of the FALP. Preliminary screens can be conducted by screening for compounds capable of binding to FALP, as at least some of the compounds so identified are likely FALP modulators. The binding assays usually involve contacting a FALP protein with one or more test compounds and allowing sufficient time for the protein and test compounds to form a binding complex. Any binding complexes formed can be detected using any of a number of established analytical techniques. Protein binding assays include, but are not limited to, methods that measure co-precipitation, co-migration on non-denaturing SDS-polyacrylamide gels, and co-migration on Western blots. The FALP protein utilized in such assays can be naturally expressed, cloned or synthesized.

In one embodiment, the assay is a cell-based assay and cells are used which are stably or transiently transfected with a vector or expression cassette having a nucleic acid sequence which encodes the FALP. The cells are maintained under conditions appropriate for expression of the FALP and are contacted with a putative agent under conditions appropriate for binding to occur. Binding can be detected using standard techniques. For example, the extent of binding can be determined relative to a suitable control (for example, relative to background in the absence of a putative agent, or relative to a known ligand).

Detection of binding or complex formation can be detected directly or indirectly. For example, the putative agent can be labeled with a suitable label (for example, fluorescent label, chemiluminescent label, isotope label, enzyme label, and the like) and binding can be determined by detection of the label.

Certain screening methods involve screening for a compound that up-regulates (or, alternatively, inhibit) the expression or activity of FALP. Such methods generally involve conducting cell-based assays in which test compounds are contacted with one or more cells expressing FALP and then detecting a change (for example, increase or decrease) in FALP expression (either transcript or translation product) or activity. Some assays are performed with cells that express endogenous FALP. Other expression assays are conducted with recombinant cells that express an exogenous FALP. In either case, FALP expression can be detected in a number of different ways, as described herein. For example, the expression level of FALP in a cell can be determined by probing the mRNA expressed in a cell with a probe that specifically hybridizes with a transcript (or complementary nucleic acid derived therefrom) of FALP. Probing can be conducted by lysing the cells and conducting Northern blots or without lysing the cells using in situ-hybridization techniques (see above). Alternatively, FALP protein can be detected using immunological methods in which a cell lysate is probe with antibodies that specifically bind to FALP. Similarly, FALP activity can be assayed. One method of drug screening utilizes eukaryotic or prokaryotic host cells which are stably transformed with recombinant DNA molecules expressing the FALP, for example, the protein having the sequence disclosed herein.

In a suitable assay, a FALP protein (whether isolated or recombinant) is used which has at least one property, activity or functional characteristic of a human FALP protein.

The level of expression or activity can be compared to a baseline value. Expression levels can also be determined for cells that do not express FALP as a negative control. Such cells generally are otherwise substantially genetically the same as the test cells.

Several methods of automating assays have been developed in recent years so as to permit screening of tens of thousands of compounds in a short period. See, for example, Fodor et al., Science 251: 767-73 (1991), and other descriptions of chemical diversity libraries, which describe means for testing of binding affinity by a plurality of compounds. The development of suitable assays can be greatly facilitated by the availability of large amounts of purified FALP and/or cells expressing recombinant FALP, as provided by this invention.

In yet another aspect, the present invention provides methods of treating FALP-mediated conditions, disorders or diseases by administering to a subject having such a disease, disorder or condition, a therapeutically effective amount of an modulator of FALP function, for example, an antagonists (inhibitors) of FALP function or gene expression. Diseases, disorders and conditions associated with FALP expression or activity include obesity and obesity-related conditions. Such modulators include small molecules agonists and antagonists of FALP function or expression; antisense and ribozyme triplex polynucleotides; gene therapy, and the like. The methods and reagents of the invention may be used in treatment of animals such as mammals (for example, humans, non-human primates, cows, sheep, goats, horses, dogs, cats, rabbits, rats, mice) or in animal or in vitro (for example, cell-culture) models of human diseases.

The present invention further provides therapeutic compositions comprising agonists, antagonists, or ligands of FALP, and methods of treating physiologic or pathologic conditions mediated by FALP.

FALP polypeptides, fragments thereof, sense and antisense polypeptides, anti-FALP antibodies or binding fragments thereof, and antagonists or agonists (for example small molecule modulators) of FALP activity, can be directly administered under sterile conditions to the host to be treated. However, while it is possible for the active ingredient to be administered alone, it is often preferable to present it as a pharmaceutical formulation. Formulations typically comprise at least one active ingredient together with one or more acceptable carriers thereof. Each carrier should be both pharmaceutically and physiologically acceptable in the sense of being compatible with the other ingredients and not injurious to the patient. For example, the bioactive agent can be complexed with carrier proteins such as ovalbumin or serum albumin prior to their administration in order to enhance stability or pharmacological properties such as half-life. Furthermore, therapeutic formulations of this invention can be combined with or used in association with other chemotherapeutic or chemopreventive agents.

Therapeutic formulations can be prepared by any methods well known in the art of pharmacy. See, for example, Gilman et al. (eds.) GOODMAN AND GILMAN'S: THE PHARMACOLOGICAL BASES OF THERAPEUTICS (8th ed.) Pcrgamon Press (1990); and Remington, THE SCIENCE OF PRACNCE AND PHARMACY, 20th Edition. (2001) Mack Publishing Co., Easton, Pa.; Avis et al. (eds.) (1993) PHARMACEUTICAL DOSAGE FORMS: PARENTERAL MEDICATIONS Dekker, N.Y.; Lieberman et al. (eds.) (1990) PHARMACEUTICAL DOSAGE FORMS: TABLETS Dekkcer, N.Y.; and Lieberman et al. (eds.) (1990) PHARMACEUTICAL DOSAGE FORMS: DISPERSE SYSTEMS Dekker, N.Y.

As used herein therefore the term “dosage forms” includes any appropriate dosage form well known in the art to be suitable for pharmaceutical formulation of proteins and/or polynucleotides suitable for administration to mammals particularly humans, particularly (although not solely) those suitable for stabilization in solution of therapeutic proteins and/or polynucleotides for administration to mammals preferably humans. All this irrespective of whether or not the FALP gene product is in the form of a composition. As used herein therefore the term “dosage forms” includes any appropriate dosage form well known in the art to be suitable for pharmaceutical formulation of proteins and/or polynucleotides suitable for administration to mammals particularly humans, particularly (although not solely) those suitable for stabilization in solution of therapeutic proteins and/or polynucleotides for administration to mammals preferably humans. All this irrespective of whether or not the FALP gene product is in the form of a composition. One example is oral delivery forms of tablet, capsule, lozenge, or the like form, or any liquid form such as syrups, aqueous solutions, emulsion and the like, capable of protecting the therapeutic protein and/or polynucleotide from degradation prior to eliciting an effect, eg; in the alimentary canal if an oral dosage form. Examples of dosage forms for transdermal delivery include transdermal patches, transdermal bandages, and the like. Included within the topical dosage forms are any lotion, stick, spray, ointment, paste, cream, gel, etc. whether applied directly to the skin or via an intermediary such as a pad, patch or the like. Examples of dosage forms for suppository delivery include any solid or other dosage form to be inserted into a bodily orifice (particularly those inserted rectally, vaginally and urethrally). Examples of dosage units for transmucosal delivery include depositories, solutions for enemas, pessaries, tampons, creams, gels, pastes, foams, nebulised solutions, powders and similar formulations containing in addition to the active ingredients such carriers as are known in the art to be appropriate. Examples of dosage units for depot administration include pellets or small cylinders of active agent or solid forms wherein the active agent is entrapped in a matrix of biodegradable polymers, microemulsions, liposomes or is microencapsulated. Examples of implantable infusion devices include any solid form in which the active agent is encapsulated within or dispersed throughout a biodegradable polymer or synthetic, polymer such as silicone, silicone rubber, silastic or similar polymer. Alternatively dosage forms for infusion devices may employ liposome delivery systems.

Depending on the disease to be treated and the subject's condition, the compounds of the present invention may be administered by oral, parenteral (for example, intramuscular, intraperitoneal, intravenous, ICV, intracistemal injection or infusion, subcutaneous injection, or implant), by inhalation spray, nasal, vaginal, rectal, sublingual, or topical routes of administration and may be formulated, alone or together, in suitable dosage unit formulations containing conventional non-toxic pharmaceutically acceptable carriers, adjuvants and vehicles appropriate for each route of administration.

The pharmaceutical composition and method of the present invention may further comprise other therapeutically active compounds as noted herein which are usually applied in the treatment of the above mentioned pathological conditions.

In the treatment or prevention of conditions which require FALP modulation an appropriate dosage level will generally be about 0.001 to 100 mg per kg patient body weight per day which can be administered in single or multiple doses. Preferably, the dosage level will be about 0.01 to about 25 mg/kg per day; more preferably about 0.05 to about 10 mg/kg per day. A suitable dosage level may be about 0.01 to 25 mg/kg per day, about 0.05 to 10 mg/kg per day, or about 0.1 to 5 mg/kg per day. Within this range the dosage may be about 0.005 to about 0.05, 0.05 to 0.5 or 0.5 to 5 mg/kg per day. For oral administration, the compositions are preferably provided in the form of tablets containing about 1 to 1000 milligrams of the active ingredient, particularly about 1, 5, 10, 15, 20, 25, 50, 75, 100, 150, 200, 250, 300, 400, 500, 600, 750, 800, 900, and 1000 milligrams of the active ingredient for the symptomatic adjustment of the dosage to the patient to be treated. The compounds may be administered on a regimen of 1 to 4 times per day, preferably once or twice per day.

It will be understood, however, that the specific dose level and frequency of dosage for any particular patient may be varied and will depend upon a variety of factors including the activity of the specific compound employed, the metabolic stability and length of action of that compound, the age, body weight, general health, sex, diet, mode and time of administration, rate of excretion, drug combination, the severity of the particular condition, and the host undergoing therapy.

The compounds of the present invention can be combined with other compounds having related utilities to prevent and treat inflammatory and immunoregulatory disorders and diseases, including asthma and allergic diseases, as well as autoimmune pathologies such as rheumatoid arthritis and atherosclerosis, and those pathologies noted above.

The present invention provides a number of methods for detection and quantification of FALP polypeptides and polynucleotides in biological samples (for example, assays for screening FALP activity or expression antagonists). In one embodiment, expression or over expression of the FALP gene product (for example, polypeptide or mRNA) is correlated with a disease or condition mediated by, or associated with the FALP.

The biological samples can include, but are not limited to, a blood sample, serum, cells (including whole cells, cell fractions, cell extracts, and cultured cells or cell lines), tissues (including tissues obtained by biopsy), body fluids (for example, urine, sputum, amniotic fluid, synovial fluid), or from media (from cultured cells or cell lines), and the like. The methods of detecting or quantifying FALP polynucleotides include, but are not limited to, amplification-based assays with or without signal amplification, hybridization based assays, and combination amplification-hybridization assays. For detecting and quantifying FALP polypeptides, an exemplary method is an immunoassay that utilizes an antibody or other binding agents that specifically binds to an FALP polypeptide or epitope.

The polymerase chain reaction (PCR), or its variations, is an exemplary amplification-based assay. Examples of techniques sufficient to direct persons of skill through in vitro amplification methods are found in PCR TECHNOLOGY: PRINCIPLES AND APPLICATIONS FOR DNA AMPLIFICATION, H. Erlich, Ed. Freeman Press, New York, N.Y. (1992); PCR PROTOCOLS: A GUIDE TO METHODS AND APPLICATIONS, eds. Innis, Gelfland, Snisky, and White, Academic Press, San Diego, Calif. (1990). Other suitable target amplification methods include the ligase chain reaction (LCR; for example, Wu and Wallace, 1989, Genomics 4: 560); strand displacement amplification (SDA; for example, Walker et al., 1992, Proc. Vatl. Acad. Sci. U.S.A. 89: 392-396); the nucleic acid sequence based amplification (NASBA, Cangene, Mississauga, Ontario; for example, Compton, 1991, Nature 350: 91), and the like. One useful variant of PCR is PCR ELISA (for example, Boehringer Mannheim Cat. No. 1 636 111) in which digoxigenin-dUTP is incorporated into the PCR product. The PCR reaction mixture is denatured and hybridized with a biotin-labeled oligonucleotide designed to anneal to an internal sequence of the PCR product. The hybridization products are immobilized on streptavidin coated plates and detected using anti-digoxigenin antibodies.

A variety of methods for specific DNA and RNA measurement using polynucleotide hybridization techniques are known to those of skill in the art (see Sambrook). Hybridization based assays refer to assays in which a polynucleotide probe is hybridized to a target polynucleotide. Usually the polynucleotide hybridization probes of the invention are entirely or substantially identical to a contiguous sequence of the FALP nucleic acid sequence. Preferably, polynucleotide probes are at least about 10 bases, often at least about 20 bases, and sometimes at least about 200 bases or more in length. Methods of selecting polynucleotide probe sequences for use in polynucleotide hybridization are discussed in Sambrook.

Polynucleotide hybridization formats are known to those skilled in the art. In some formats, at least one of the target and probe is immobilized. The immobilized polynucleotide may be DNA, RNA, or another oligo- or poly-nucleotide, and may comprise natural or non-naturally occurring nucleotides, nucleotide analogs, or backbones. Such assays may be in any of several formats including: Southern, Northern, dot and slot blots, high-density polynucleotide or oligonucleotide arrays (for example, GeneChips™ Affymetrix), dip sticks, pins, chips, or beads. All of these techniques are well known in the art and are the basis of many commercially available diagnostic kits. Hybridization techniques are generally described in Hames et al., ed., NUCLEIC ACID HYBRIDIZATION, A PRACTICAL APPROACH IRL Press, (1985); Gall and Pardue Proc. Natl. Acad. Sci., U.S.A., 63: 378-383 (1969); and John et al., Nature, 223: 582-587 (1969).

In one embodiment, in situ hybridization is used to detect FALP sequences in a sample. In situ hybridization assays are well known and are generally described in Angerer et al., METHODS ENZYMOL., 152: 649-660 (1987) and Ausubel, supra.

In one embodiment, the FALP polynucleotide is detected in a sample using an anti-FALP antibody of the invention. A number of well established immunological binding assay are suitable for detecting and quantifying FALP of the present invention. See, for example, U.S. Pat. Nos. 4,366,241; 4,376,110; 4,517,288; and 4,837,168, and also METHODS IN CELL BIOLOGY VOLUME 37: ANTIBODIES IN CELL BIOLOGY, Asai, ed. Academic Press, Inc. New York (1993); BASIC AND CLINICAL IMMUNOLOGY 7th Edition, Stites & Terr, eds. (1991); Harlow, supra [for example, Chapter 14], and Ausubel, supra, [for example, Chapter 11].

Immunoassays for detecting FALP may be competitive or noncompetitive. Usually the FALP gene product being assayed is detected directly or indirectly using a detectable label. The particular label or detectable group used in the assay is usually not a critical aspect of the invention, so long as it does not significantly interfere with the specific binding of the antibody or antibodies used in the assay. The label may be covalently attached to the capture agent (for example, an anti-FALP antibody), or may be attached to a third moiety, such as another antibody, that specifically binds to the FALP polypeptide at a different epitope than recognized by the capture agent.

Noncompetitive immunoassays are assays in which the amount of captured analyte (here, the FALP polypeptide) is directly measured. One such assay is a two-site, monoclonal-based immunoassay utilizing monoclonal antibodies reactive to two non-interfering epitopes on the captured analyte. See, for example, Maddox et al., J. Exp. Med., 158: 1211 (1983) for background information. In such an assay, the amount of FALP in the sample is directly measured For example, using a so-called “sandwich” assay, the capture agent (here, the anti-FALP antibodies) can be bound directly to a solid substrate where they are immobilized. These immobilized antibodies then capture polypeptide present in the test sample. FALP thus immobilized is then bound by a labeling agent, such as a second FALP antibody bearing a label. Alternatively, the second FALP antibody may lack a label, but it may, in turn, be bound by a labeled third antibody specific to antibodies of the species from which the second antibody is derived. The second can be modified with a detectable moiety, such as biotin, to which a third labeled molecule can specifically bind, such as enzyme-labeled streptavidin. Negative and positive controls (current or historic) may also be utilized.

In competitive assays, the amount of FALP polypeptide present in the sample is measured indirectly by measuring the amount of an added (exogenous) FALP displaced (or competed away) from a capture agent (for example, anti-FALP antibody) by the analyte present in the sample (for example, FALP polypeptide). In one competitive assay, a known amount of FALP is added to the sample and the sample is then contacted with a capture agent (for example, an anti-FALP antibody) that specifically binds to FALP. The amount of FALP bound to the antibody is inversely proportional to the concentration of FALP present in the sample.

Preferably, the antibody is immobilized on a solid substrate. The amount of FALP bound to the antibody may be determined either by measuring the amount of FALP present in an FALP/antibody complex, or alternatively by measuring the amount of remaining uncomplexed FALP. The amount of FALP may be detected by providing a labeled FALP molecule.

For example, using the hapten inhibition assay, the analyte (in this case FALP) is immobilized on a solid substrate. A known amount of anti-FALP antibody is added to the sample, and the sample is then contacted with the immobilized FALP. In this case, the amount of anti-FALP antibody bound to the immobilized FALP is inversely proportional to the amount of FALP present in the sample. Again the amount of immobilized antibody may be detected by detecting either the immobilized fraction of antibody or the fraction of the antibody that remains in solution. Detection may be direct where the antibody is labeled or indirect by the subsequent addition of a labeled moiety that specifically binds to the antibody as described above.

In addition to the competitive and non-competitive FALP polypeptide immunoassays, the present invention also provides other assays for detection and quantification of FALP polypeptides. For example, Western blot (immunoblot) analysis can be used to detect and quantify the presence of FALP in the sample. The technique generally comprises separating sample polypeptides by gel electrophoresis on the basis of molecular weight, transferring the separated polypeptides to a suitable solid support (such as a nitrocellulose filter, a nylon filter, or derivatized nylon filter), and incubating the sample with the antibodies that specifically bind FALP. The anti-FALP antibodies specifically bind to FALP on the solid support. These antibodies may be directly labeled or alternatively may be subsequently detected using labeled antibodies (for example, labeled sheep anti-mouse antibodies) that specifically bind to the anti-FALP.

Furthermore, assays such as liposome immunoassays (LIA) are also encompassed by the present invention. LIA utilizes liposomes that are designed to bind specific molecules (for example, antibodies) and to release encapsulated reagents or markers. The released chemicals are then detected according to standard techniques (see, Monroe et al., 1986, Amer. Clin. Prod. Rev. 5: 3441).

Reagents useful for the therapeutic and diagnostic (detection) methods of the invention are conveniently provided in kit form. Thus, the present invention encompasses kits that contain polypeptides, antibodies, and polynucleotides of the present invention.

In one embodiment, the kit comprises one or more of the following in a container: (1) FALP polynucleotides (for example, oligonucleotide primers or probes corresponding to the FALP cDNA sequence and capable of amplifying the target polynucleotides); (2) anti-FALP antibodies; (3) FALP polypeptides or fragments, optionally coated on a solid surface (such as a slide, multiple well plate, or test tube) (4) a FALP polynucleotide (for example, for use as positive controls in assays), (5) and tubes. Instructions for carrying out the detection methods of the invention, and calibration curves can also be included.

### EXAMPLES

Material and Methods

Differentiation of 3T3-L1 cells —3T3-L1 preadipocytes were maintained as subconfluent cultures in DMEM supplemented with 10% fetal calf serum. Differentiation of postconfluent cells was initiated by treatment with 0.25 μM dexamethasone, 0.5 mM IBMX and 10 μg/ml insulin for 2 days. This is followed by incubation with 10 μg/ml insulin for 2 days. The cells were then maintained in DMEM with 10% fetal calf serum for another 4 days. Red Oil 0 staining revealed that over 90% of cells exhibit typical morphology of adipocytes.

Two dimensional gel electrophoresis(2-DE)-3T3 L1 preadipocytes or adipocytes (day 8 after differentiation) were solubilized in a lysis buffer containing 7M urea, 2M thiourea, 4% CHAPS, 10 mM DTT, 1% Pharmalyte 3-10. Equal amount of proteins from these samples was then analysed by two dimensional gel electrophoresis as described previously (Wang, Y., Xu, A., Ye, J., Kraegen, E. W., Tse, C. A. and Cooper, G. J. (2001) Diabetes 50, 1821-7), using pH 47 immobiline Drystrips (Pharmacia) for first dimensional focussing. The separated proteins were stained with either silver or Coomassie Brilliant Blue R250 (CBB). The differentially expressed proteins were identified by Melanine 2 software (Bio-Rad).

In-gel trypsin digestion, reversed phase high performance liquid chromatography (RP HPLC) and amino acid sequencing—Proteins of interest separated by 2-DE gels were excised, and gel pieces were subjected to in-gel trypsin digestion as described previously. Xu, A., Bellamy, A. R. and Taylor, J. A., EMBO Journal 19, 6465-74 (2000). The extracted tryptic peptide mixtures were fractionated by RP HPLC on a Jupiter 5μ C18 column (250×2.00 mm, Phenomenex). The pre-warmed column (37° C.) was washed for 7 min with 0.1% trifluoroacetic acid (v/v) followed by elution using a 50 min linear gradient from 8% to 36% of acetonitrile at the flow rate of 200 μl/min. The well-separated fractions were chosen for amino acid sequencing using the Edman degradation method with a Perkin-Elmer (Procise, Model 492) protein sequencer.

Cloning of human and mouse FALP—Total RNA was purified from mouse 3T3-L1 adipocytes or human fat pads using TRIZOL reagent according to the manufacturer's instructions (Life technology). The oligo-dT-primed cDNA from the total RNA was used as a template for degenerate PCR cloning. The two degenerate primers, which were designed according to the amino acid sequences of the two tryptic peptides of mouse FALP, are

| | | |
|---|---|---|
| 1. | 5′-ATGGCNAAYGGNACNGAYGCNAGY-3′ | (SEQ ID NO:1) |
| 2. | 5′-YTGNGTNAYCCAYTGRTCNGTNCCNGC-3′ | (SEQ ID NO:2) |

The following two “guessmers” were used for cloning of human FALP:

(SEQ ID NO:3)

| | |
|---|---|
| 1. | hFALP/UP: 5′-ATCGGGATCCATGGCCAACGGGACCAAC-3′ |
| | (sense), |

(SEQ ID NO:4)

| | |
|---|---|
| 2. | hFALP/DS: 5′-GTACGAATTCCCTCATCTGCGGGGAGGC-3′ |
| | (antisense). |

The full length cDNAs of mouse and human FALP were obtained using 3′- and 5′-rapid amplification of cDNA end (RACE) according to manufacturer's instruction (Life Technology). The DNA fragments were then inserted into pGEMT-easy vector (Bromega) for DNA sequence verification.

The vector for mammalian expression of mouse FALP was generated by cDNA amplification using the sense primer 5′-ATCGGGATCCATGGCCAACGGGACCGACGCC-3′(SEQ ID NO:5) and the antisense primer 5′ATCGGAATTCTCACTTGTCATCGTCGTCCTTGTAGTCGGGGAGAGCCAGGGGCC-3′(SEQ ID NO:6). Following digestion with BamHI/EcoRI, the fragment was inserted into pcDNA3.1 vector to produce pcDNA-FALP-F, which encodes full-length FALP with FLAG epitope tagged at its C-terminus.

Transient expression of mouse FALP and immunocytochemistry—The mammalian expression vector encoding FLAG-tagged FALP was transfected into COS-7 cells or 3T3 L1 adipocytes using FuGENE 6 transfection reagent. The cells were allowed to grow for another 24 hr or 48 hr, fixed using methanol/acetone, incubated with mouse anti-FLAG monoclonal antibody (Sigma), and then stained with cy3-conjugated goat anti mouse polyclonal antibody (Sigma). The speciman were then analysed using ZEISS Axioskop 2 plus microscopy equipped with a digitalized camera.

Northern blot analysis —10 μg of total RNA purified from either 3T3 L1 cells or mouse adipose tissue was separated 1.2% formaldehyde-denaturing agarose gel and transferred to Nylon membranes. Hybridization was carried out as described previously (Xu, A., Bellamy, A. R. and Taylor, J. A. (1999) Biochemical Journal 342, 683-9), using ³²P labelled full-length adipocyspin, adiponectin, PPAR y or GLUT4 cDNA as a probe. The membranes were visualized using a phosphorimager and quantitated using MacBAS software (Fujifilm).

Results and Discussion

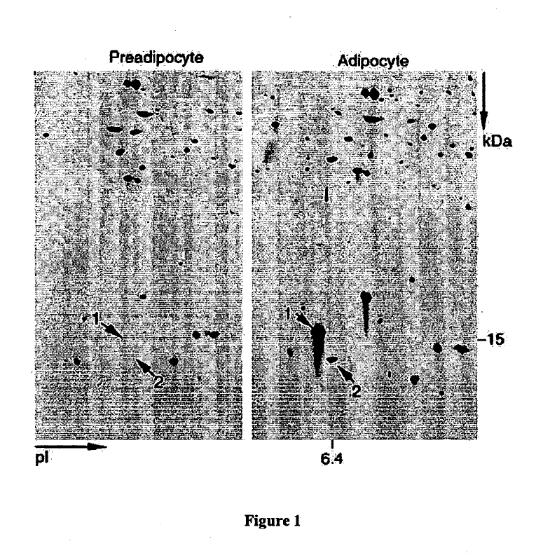

Characterization of FALP, a novel protein expressed during adipose conversion —To identify novel proteins that are differentially expressed during adipose conversion, proteins from 3T3 L1 preadipocytes and adipocytes were analyzed by 2-DE. Within the low molecular weight region of the gels, we found two proteins which were selectively expressed in adipocytes (day 8 after differentiation), and not in preadipocytes (FIG. 1).

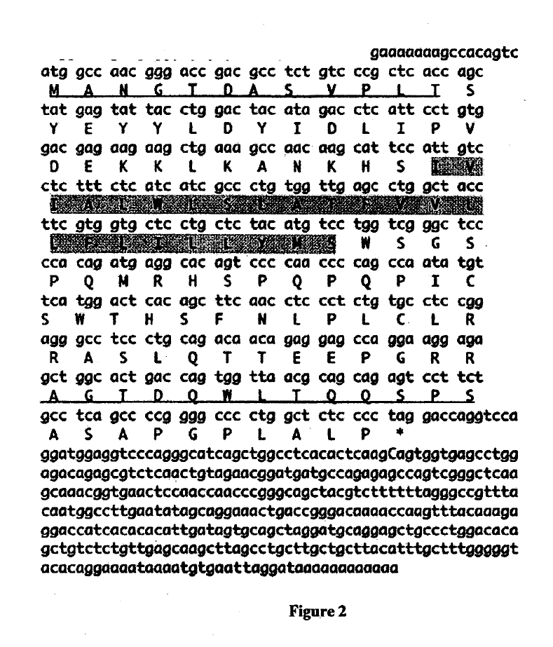

To identify the nature of these two proteins, the spots were excised from multiple preparative 2D gels and in gel digested by trypsin. The tryptic peptide mixture of the protein was separated by RP HPLC and the well-resolved fractions were analysed by amino acid sequencing. Database searching revealed that a sequenced peptide (FDETTAD) derived from spot 1 matched exclusively with mouse epidermal fatty acid-binding protein (Swissprot Accession number P55053). The amino acid sequences for the tryptic peptides MANGTDASVPLT (SEQ ID NO:7) and AGTDQWLTQQSPS (SEQ ID NO:8) derived from “spot” 2 did not show significant homology with any known proteins present in the available database. The partial cDNA sequence of this protein was obtained by degenerate RT PCR using the primers which were designed according to the amino acid sequence of the tryptic peptides. The entire coding cDNA sequence was obtained by 3′- and 5′-RACE PCR strategy (FIG. 2). The DNA sequence of this gene was deposited in Genebank (accession number AY079153). BLASTn searching of the nucleic acid database at the National Center for Biotechnology Information revealed that the cDNA sequence of mouse FALP shares significant homology with an expressed sequence tag (EST) sequence derived from Mus musculus 18 days embryo cDNA (gene accession number: AK003912). The open reading frame of the cDNA sequence of mouse FALP encodes a putative protein with the predicated molecular mass of about 14 kDa and a pI value of about 6.4 (FIG. 2), which perfectly matches to the protein spot separated within the 2D gel (FIG. 1). BLASTp database searching showed the full-length amino acid sequence of FALP has little homology with any known proteins, suggesting that FALP is a novel adipocyte protein.

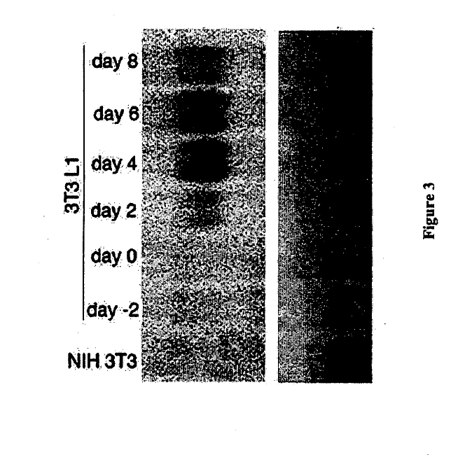

Differentiation-dependent expression of FALP mRNA—We next examined the time course of FALP mRNA expression during the adipose conversion of 3T3 L1 preadipocytes by Northern blot analysis. As shown in FIG. 3, FALP mRNA correlate well with cell differentiation and changes in cell morphology (round shape and appearance of intracellular lipid droplets). The FALP mRNA with the mass of approximately 800 bp started to appear as early as day 2 following induction of adipose conversion, and reached to the maximum at day 6. The expression kinetics of FALP paralleled those of aP2, and slightly earlier than adiponectin, a protein exclusively expressed in adipocytes. Scherer, P. E., Williams, S., Fogliano, M., Baldini, G. and Lodish, H. F., Journal of Biological Chemistry 270, 26746-26749 (1995); Hu, E., Liang, P. and Spiegelman, B. M., Journal of Biological Chemistry 271, 10697-10703 (1996). Thus, our results demonstrated a simultaneous appearance of FALP mRNA expression and adipocyte phenotype.

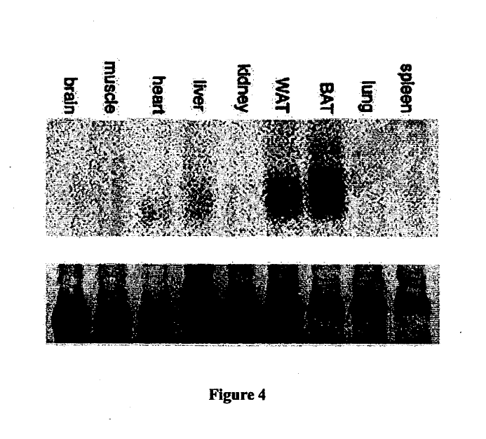

Adipose tissue-specific expression of FALP in mouse—To investigate the tissue distribution of FALP, we performed Northern analysis using various tissue RNAs from mice. As shown in FIG. 4, FALP mRNA was predominantly expressed in mouse epididymal (white adipose tissue) and interscapular (brown adipose tissue) fat pads. The size of FALP mRNAs in adipose tissue was similar to those detected in 3T3 L1 adipocytes. FALP mRNA was hardly detected in other tested tissues, including lung, spleen, kidney, intestine, heart and skeletal muscle and liver. This analysis indicated that expression of both isoforms of FALP was largely restricted to fat tissue in mice.

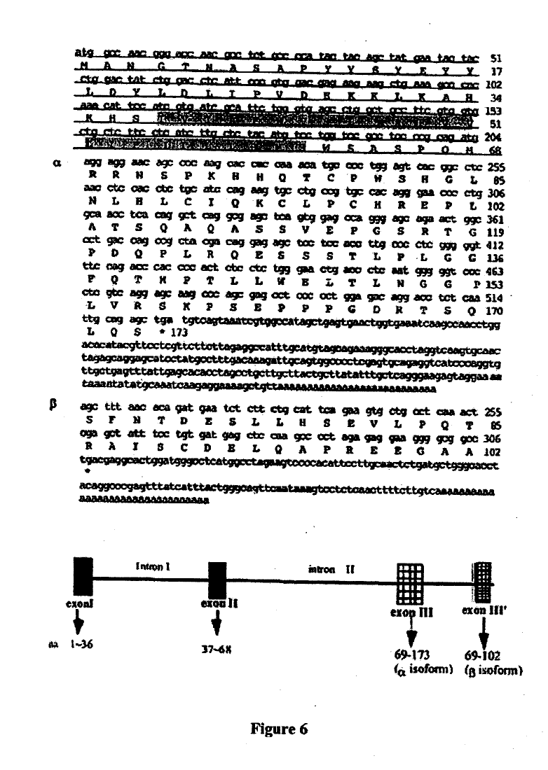

Human homologs of FALP exist as two alternatively spliced isoforms—To identify the human homologs of mouse FALP, its amino acid sequence was used to search against human genomic sequences. This analysis found that there are two DNA fragments within chromosome 21q22.1 region, which were intercepted by 7670 base pairs, and encode a potential polypeptide which has over 85% identity with N-terminal 69 amino acid residues of mouse FALP. To confirm the existence of this potential human FALP homolog in adipose tissue, two “guessmers” were designed based on the potential sequence of human FALP gene, as described in the Methods. Reverse transcription PCR analysis using the two “guessmers” revealed a band with the expected size (˜180 bp) (FIG. 5). DNA sequence analysis verified the expression of this putative gene. Further analysis using 3′ rapid amplification of cDNA end found that the gene of human FALP existed as two distinct isoforms, which are termed human FALP α and FALP β respectively (FIG. 6). The DNA sequences of these two genes were deposited in Genebank (accession number: AY079152 and AF483549). The encoding region of these two isoforms shares a common N-terminus, but have distinct C-termini. The conceptual protein of human FALP α and β is composed of 173 and 102 amino acid residues, with the predicted molecular mass of about 19 kDa and about 11 kDa, respectively. The gene structure of human FALP contains three exons, with exon I and II encoding the NH₂-terminal 68 amino acid residues shared by both isoforms (FIG. 5). The exon III and II′ are alternatively spiced, which encodes the COOH-terminal 105 amino acid residues for a isoform and 33 amino acid residues for β isoform, respectively.

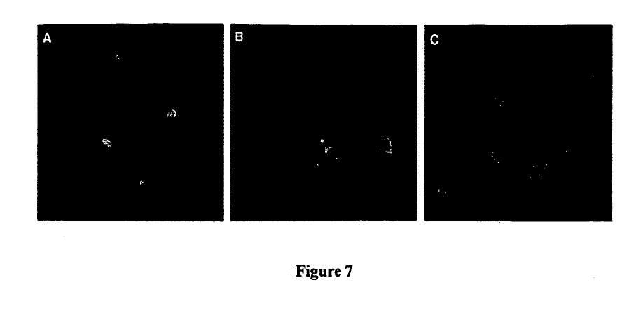

Subcellular distribution of FALP—Sequence analysis of human and mouse FALPs using the program TMpred (Hofmann, K. and Stoffel, W., Biol. Chem. Hoppe-Seyler 374, 166 (1993)) identified a conserved single transmembrane domain spanning 23 amino acid residues, indicating that FALP might be an integral membrane protein. In order to investigate the intracellular localization of FALP, a COOH-terminal FLAG epitope-tagged FALP construct was introduced into either COS 7 cells or 3T3 L1 adipocytes by transient transfection. Analysis using an immunofluroscent microscopy revealed that the preponderance of FLAG-tagged FALP accumulated at the perinuclear region where it forms a compact patch-like structure (FIGS. 7A and 7B). Treatment of 3T3 L1 adipocytes with 50 nM insulin caused the redistribution of FALP from the compact perinuclear compartment into numerous discrete spotty structures spreading throughout the cytoplasm (FIG. 7C). A few spotty structures could also be observed at the peripheral region of the cells, implicating its presence at the plasma membrane. This result suggests that FALP is localized at a dynamic compartment which is regulated by insulin. Insulin treatment did not cause redistribution of FALP in COS 7 cells, perhaps due to the lack of insulin receptor or other signaling components in this cell line.

All patents, publications, scientific articles, web sites, and other documents and materials referenced or mentioned herein are indicative of the levels of skill of those skilled in the art to which the invention pertains, and each such referenced document and material is hereby incorporated by reference to the same extent as if it had been incorporated by reference in its entirety individually or set forth herein in its entirety. Applicants reserve the right to physically incorporate into this specification any and all materials and information from any such patents, publications, scientific articles, web sites, electronically available information, and other referenced materials or documents.

The specific methods and compositions described herein are representative of preferred embodiments and are exemplary and not intended as limitations on the scope of the invention. Other objects, aspects, and embodiments will occur to those skilled in the art upon consideration of this specification, and are encompassed within the spirit of the invention as defined by the scope of the claims. It will be readily apparent to one skilled in the art that varying substitutions and modifications may be made to the invention disclosed herein without departing from the scope and spirit of the invention. The invention illustratively described herein suitably may be practiced in the absence of any element or elements, or limitation or limitations, which is not specifically disclosed herein as essential. Thus, for example, in each instance herein, in embodiments or examples of the present invention, any of the terms “comprising”, “consisting essentially of”, and “consisting of” may be replaced with either of the other two terms in the specification. Also, the terms “comprising”, “including”, containing”, etc. are to be read expansively and without limitation. The methods and processes illustratively described herein suitably may be practiced in differing orders of steps, and that they are not necessarily restricted to the orders of steps indicated herein or in the claims. It is also that as used herein and in the appended claims, the singular forms “a,” “an,” and “the” include plural reference unless the context clearly dictates otherwise. Thus, for example, a reference to “a host cell” includes a plurality (for example, a culture or population) of such host cells, and so forth. Under no circumstances may the patent be interpreted to be limited to the specific examples or embodiments or methods specifically disclosed herein. Under no circumstances may the patent be interpreted to be limited by any statement made by any Examiner or any other official or employee of the Patent and Trademark Office unless such statement is specifically and without qualification or reservation expressly adopted in a responsive writing by Applicants.

The terms and expressions that have been employed are used as terms of description and not of limitation, and there is no intent in the use of such terms and expressions to exclude any equivalent of the features shown and described or portions thereof, but it is recognized that various modifications are possible within the scope of the invention as claimed. Thus, it will be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

Other embodiments are within the following claims. In addition, where features or aspects of the invention are described in terms of Markush groups, those skilled in the art will recognize that the invention is also thereby described in terms of any individual member or subgroup of members of the Markush group.

1. A method for treatment of obesity, obesity-related conditions, and conditions related to intracellular trafficking pathways of adipose cells including GLUT4 translocation and hormone secretion, insulin resistance, type II diabetes, hypertension, dyslipidemia, and/or other conditions related to insulin resistance and/or syndrome X, by down regulating the expression or activity of a FALP.

2. A method of screening for agents useful for modulating a FALP comprising contacting a composition comprising a with an agent and detecting a change in activity or expression of the FALP in response to the agent.

3. An isolated polynucleotide that encodes, or is complementary to a sequence that encodes, a FALP polypeptide.

4. A polynucleotide of claim 3 that is operably linked to a promoter or other sequence that enhances expression of the polynucleotide in a cell.

5. A recombinant vector capable of expressing a FALP polypeptide or fragment thereof.

6. A cell, including but not limited to a bacterial, eukaryotic, mammalian, or human cell, containing a recombinant FALP polynucleotide.

7. A method for producing an FALP protein, peptide, or fusion protein by culturing a cell containing the recombinant FALP polynucleotide under conditions in which the polypeptide is expressed.

8. An isolated, substantially pure, synthetic or recombinant FALP polypeptide, or immunogenic or functional fragment or variant thereof.

9. A polypeptide of claim 8 that has an amino acid sequence selected from the group consisting of SEQ ID NO: 1, SEQ ID NO:14, SEQ ID NO:17 and SEQ ID NO:20.

10. An isolated polypeptide with an amino acid sequence that differs by conservative mutations from an amino acid sequence selected from the group consisting of SEQ ID NO:11, SEQ ID NO:14, SEQ ID NO:17 and SEQ ID NO:20, which is at least 60% identical to the reference sequence and/or that is immunologically cross-reactive with the full-length naturally occurring FALP polypeptide.

11. An isolated polypeptide with an amino acid sequence that differs by conservative mutations from an amino acid sequence selected from the group consisting of SEQ ID NO:11, SEQ ID NO:14, SEQ ID NO:17 and SEQ ID NO:20, which is at least 80% identical to the reference sequence and/or that is immunologically cross-reactive with the full-length naturally occurring FALP polypeptide.

12. An isolated polypeptide with an amino acid sequence that differs by conservative mutations from an amino acid sequence selected from the group consisting of SEQ ID NO:11, SEQ ID NO:14, SEQ ID NO:17 and SEQ ID NO:20, which is at least 90% identical to the reference sequence and/or that is immunologically cross-reactive with the full-length naturally occurring FALP polypeptide.

13. A FALP polypeptide or immunogenic or functional fragment thereof that is a fusion protein.

14. An isolated antibody, or antibody fragment that specifically binds to the FALP polypeptide of the invention.

15. An antibody of claim 14 that is monoclonal.

16. An antibody of claim 14 or 15 that binds with an affinity of at least about 10⁸ M⁻¹.

17. An antibody of claim 14 that is human or humanized.

18. An isolated cell or a hybridoma capable of secreting the antibody of claim 14.

19. A method of detecting an FALP gene product in a sample by (a) contacting the sample with a probe or primer that specifically binds the gene product, wherein the probe or primer and the gene product form a complex, and detecting the formation of the complex; or (b) specifically amplifying the gene product in the biological sample, wherein said gene product is a polynucleotide, and detecting the amplification product; wherein the formation of the complex or presence of the amplification product is correlated with the presence of the FALP gene product in the biological sample.

20. A method according to claim 19 wherein the gene product is a polypeptide and the probe is an antibody.

21. A method according to claim 19 wherein the gene product is an RNA and the probe or primer is a polynucleotide.

22. A method of identifying a modulator of FALP activity by contacting a composition comprising FALP and a test compound and assaying for a biological effect that occurs in the presence but not absence of the test compound, wherein a test compound that induces a biological effect is identified as a modulator of FALP activity.

23. A method according to claim 22 wherein the composition comprising FALP is a cell expressing a FALP polypeptide.

24. A method according to claim 23 wherein the composition comprising FALP is a cell expressing a recombinant FALP polypeptide.

25. A method according to claim 22 wherein the composition comprising FALP is a cell expressing a FALP polypeptide and the biological effect is the redistribution of the FALP polypeptide elicited by insulin.

26. A method according to claim 25 wherein the composition comprising FALP is a 3T3 L1 adipocyte expressing a FALP polypeptide and the biological effect is the redistribution of the FALP polypeptide elicited by insulin.

27. A process for making a pharmaceutical composition by formulating a modulator of FALP activity for pharmaceutical use.

28. A process for making a pharmaceutical composition by formulating a modulator of FALP activity for pharmaceutical use, wherein said modulator is identified by the method of claim 22.

29. A method for identifying compounds which will be useful for the treatment of FALP-mediated diseases and conditions, by determining whether the compound interacts with a FALP gene product.

30. A method according to claim 29 wherein the FALP gene product is a polypeptide and interaction is determined by biochemical or physical means.

31. A method of treating an FALP-mediated condition such as but not limited to obesity, obesity related conditions, and conditions related to intracellular trafficking pathways of adipose cells such as GLUT4 translocation and hormone secretion, insulin resistance, type II diabetes, hypertension, dyslipidemia, and/or other conditions related to insulin resistance and/or syndrome X, in a mammal by reducing or increasing the activity or expression of FALP in a cell or tissue in the mammal or administering a modulator of FALP activity to the mammal.

32. The use of a FALP gene product together with any pharmaceutically acceptable excipients, diluents, carriers, co-actives, or the like in the preparation of a medicament suitable for treatment of obesity, obesity related conditions, and conditions related to intracellular trafficking pathways of adipose cells such as GLUT4 translocation and hormone secretion, insulin resistance, type II diabetes, hypertension, dyslipidemia, and/or other conditions related to insulin resistance and/or syndrome X.

33. The use according to claim 32 wherein the FALP gene product is a FALP polypeptide, or immunogenic or functional fragment or variant thereof.

34. The use of according to claim 33 wherein the FALP polypeptide has the amino acid sequence identical to one disclosed herein.

35. The use of an isolated polynucleotide that encodes or is complimentary to a sequence that encodes a FALP polypeptide together with any pharmaceutically acceptable excipients, valuents, carriers, co-actives, or the like in the preparation of a medicament suitable for the of treatment of obesity, obesity related conditions, and conditions related to intracellular trafficking pathways of adipose cells such as GLUT4 translocation and hormone secretion, insulin resistance, type II diabetes, hypertension, dyslipidemia, and/or other conditions related to insulin resistance and/or syndrome X.

36. A polynucleotide selected from the group consisting of an antisense, triplex, ribozyme, or RNA-i polynucleotide, capable of targeting or hybridizing a FALP polynucleotide.

37. The use according to claim 35 wherein the polynucleotide is an antisense, triplex, ribozyme or RNA-1 polynucleotide.

38. The use of an inhibitory polynucleotide selected from the group consisting of an antisense, triplex, ribozyme, or RNA-i polynucleotides capable of targeting or hybridizing two FALP polynucleotides together with pharmaceutically acceptable excipients, diluents, carriers, or co-actives, in the preparation of a medicament suitable for treatment of obesity, obesity related conditions, and conditions related to intracellular trafficking pathways of adipose cells such as GLUT4 translocation and hormone secretion, insulin resistance, type IT diabetes, hypertension, dyslipidemia, and/or other conditions related to insulin resistance and/or syndrome X.

39. The use according to claim 38 wherein the polynucleotide is an antisense or triplex polynucleotide.

40. The use of an antibody or antibody fragment or binding fragment capable of specifically binding to a FALP together with any pharmaceutically acceptable excipients, diluents, carriers, co-actives, or the like in the preparation of a medicament suitable for the treatment of obesity treatment of obesity, obesity related conditions, and conditions related to intracellular trafficking pathways of adipose cells such as GLUT4 translocation and hormone secretion, insulin resistance, type II diabetes, hypertension, dyslipidemia, and/or other conditions related to insulin resistance and/or syndrome X.

41. The use of a modulator of FALP activity together with any pharmaceutically acceptable excipients, co-actives, diluents, carrier on the like, in the preparation of a medicament suitable for treatment of obesity, obesity related conditions, and conditions related to intracellular trafficking pathways of adipose cells such as GLUT4 translocation and hormone secretion, insulin resistance, type II diabetes, hypertension, dyslipidemia, and/or other conditions related to insulin resistance and/or syndrome X.

42. The use of an effective amount of a FALP gene product in the manufacture with other material or materials (whether excipients, co-actives, diluents or the like and/or whether a dosage unit defining vessel) of a dosage unit effective for use in the treatment of treatment of obesity, obesity related conditions, and conditions related to intracellular trafficking pathways of adipose cells such as GLUT4 translocation and hormone secretion, insulin resistance, type II diabetes, hypertension, dyslipidemia, and/or other conditions related to insulin resistance and/or syndrome X.

43. The use according to claim 40 wherein the FALP gene product is a FALP polypeptide or immunogenic or functional fragment or variant thereof.

44. The use of an effective amount of a modulator of FALP activity in the manufacture with other material or materials (whether excipients, co-actives, diluents or the like and/or whether a dosage unit defining vessel) of a dosage unit effective for use in the treatment of obesity, obesity related conditions, and other conditions related to intracellular trafficking pathways of adipose cells such as GLUT4 translocation and hormone secretion, insulin resistance, type II diabetes, hypertension, dyslipidemia, and/or other conditions related to insulin resistance and/or syndrome X.

45. An article of manufacture comprising or including a vessel containing a FALP gene product and/or a modulator of FALP activity; instructions for use of the FALP gene product and/or modulator of FALP activity effective for use in the treatment of obesity, obesity related conditions, and conditions related to intracellular trafficking pathways of adipose cells such as GLUT4 translocation and hormone secretion, insulin resistance, type II diabetes, hypertension, dyslipidemia, and/or other conditions related to insulin resistance and/or syndrome X.

46. A dosage unit useful in the treatment of obesity, obesity related conditions, and conditions related to intracellular trafficking pathways of adipose cells such as GLUT4 translocation and hormone secretion, insulin resistance, type II diabetes, hypertension, dyslipidemia, and/or other conditions related to insulin resistance and/or syndrome X, the dosage unit being an effective amount of a FALP gene product and/or a modulator of FALP activity together with any suitable pharmaceutically acceptable excipients, diluent, carrier, co-active and/or any appropriate vessel for said effective amount.

47. A product of the process of claim 22 that modulates FALP.

48. A FALP immunoassay.

49. A FALP probe.

50. A FALP primer.

51. A FALP cloning vector.
